(19) United States
(12) Patent Application Publication
(10) Pub. No.: US 2005/0037946 A1
(43) Pub. Date: Feb. 17, 2005

(54) Methods and compositions for treating cardiovascular disease using 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419

(75) Inventors: Nancy E. Stagliano, North Reading, MA (US); Aileen Healy, Medford, MA (US); Susan L. Acton, Lexington, MA (US); Katherine M. Galvin, Jamaica Plain, MA (US); Mary A. Donoghue, Belmont, MA (US); Amelie Rodrigue-Way, Lasalle (CA); James E. Tomlinson, Burlingame, CA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 10/753,267

(22) Filed: Jan. 8, 2004

Related U.S. Application Data
(60) Provisional application No. 60/439,683, filed on Jan. 13, 2003.
(60) Provisional application No. 60/445,216, filed on Feb. 5, 2003.
(60) Provisional application No. 60/448,036, filed on Feb. 18, 2003.
(60) Provisional application No. 60/454,189, filed on Mar. 12, 2003.
(60) Provisional application No. 60/457,541, filed on Mar. 25, 2003.
(60) Provisional application No. 60/466,411, filed on Apr. 29, 2003.
(60) Provisional application No. 60/469,041, filed on May 8, 2003.
(60) Provisional application No. 60/477,414, filed on Jun. 10, 2003.
(60) Provisional application No. 60/478,560, filed on Jun. 13, 2003.
(60) Provisional application No. 60/489,772, filed on Jul. 24, 2003.
(60) Provisional application No. 60/490,660, filed on Jul. 28, 2003.
(60) Provisional application No. 60/499,838, filed on Sep. 3, 2003.
(60) Provisional application No. 60/504,786, filed on Sep. 22, 2003.
(60) Provisional application No. 60/505,570, filed on Sep. 24, 2003.
(60) Provisional application No. 60/512,418, filed on Oct. 17, 2003.
(60) Provisional application No. 60/514,660, filed on Oct. 27, 2003.

(51) Int. Cl. A61K 31/00

(52) U.S. Cl. 514/1

(57) ABSTRACT

The present invention relates to methods for the diagnosis and treatment of cardiovascular disease, including, but not limited to, atherosclerosis, reperfusion injury, hypertension, restenosis, arterial inflammation, thrombosis and endothelial cell disorders. Specifically, the present invention identifies the differential expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 and 2419 genes in cardiovascular disease states, relative to their expression in normal, or non-cardiovascular disease states, and/or in response to manipulations relevant to cardiovascular disease. The present invention describes methods for the diagnostic evaluation and prognosis of various cardiovascular diseases, and for the identification of subjects exhibiting a predisposition to such conditions. The invention also provides methods for identifying a compound capable of modulating cardiovascular disease. The present invention also provides methods for the identification and therapeutic use of compounds as treatments of cardiovascular disease.

### RELATED APPLICATIONS

The present application claims the benefit of U.S. Provisional Application Ser. No. 60/439,683, filed on Jan. 13, 2003, of U.S. Provisional Application Ser. No. 60/445,216, filed on Feb. 5, 2003, of U.S. Provisional Application Ser. No. 60/448,036, filed on Feb. 18, 2003, of U.S. Provisional Application Ser. No. 60/454,189, filed on Mar. 12, 2003, of U.S. Provisional Application Ser. No. 60/457,541, filed on Mar. 25, 2003, of U.S. Provisional Application Ser. No. 60/466,411, filed on Apr. 29, 2003, of U.S. Provisional Application Ser. No. 60/469,041, filed on May 8, 2003, of U.S. Provisional Application Ser. No. 60/477,414, filed on Jun. 10, 2003, of U.S. Provisional Application Ser. No. 60/478,560, filed on Jun. 13, 2003, of U.S. Provisional Application Ser. No. 60/489,772, filed on Jul. 24, 2003, of U.S. Provisional Application Ser. No. 60/490,660, filed on Jul. 28, 2003, of U.S. Provisional Application Ser. No. 60/499,838, filed on Sep. 3, 2003, of U.S. Provisional Application Ser. No. 60/504,786, filed on Sep. 22, 2003, of U.S. Provisional Application Ser. No. 60/505,570, filed on Sep. 24, 2003, of U.S. Provisional Application Ser. No. 60/512,418, filed on Oct. 17, 2003 and of U.S. Provisional Application Ser. No. 60/514,660, filed on Oct. 27, 2003. The entire contents of these provisional patent applications are hereby incorporated in their entirety by this reference.

### BACKGROUND OF THE INVENTION

Cardiovascular disease is a major health risk throughout the industrialized world. Atherosclerosis, the most prevalent of cardiovascular diseases, is the principal cause of heart attack, stroke, and peripheral vascular disease resulting in significant disability and limb loss, and thereby the principle cause of death in the United States.

Atherosclerosis is a complex disease involving aspects of lipid metabolism and vascular inflammation. Both have significant effects on the initiation and progression of atherosclerosis. Irregular lipid metabolism, is a very well established risk factor for atherosclerosis. Elevated low density lipoprotein (LDL), very low density lipoproteins (VLDL), triglycerides and low levels of high density lipoproteins (HDL) all independently contribute to atherosclerosis development and/or progression. There are a number of effective therapies currently being utilized in the clinic that result in lowering of these risk factors and, in turn decrease the rate of mortality and morbidity associated with atherosclerotic disease. Some of these therapies include the cholesterol lowering drugs statins, the triglyceride lowering drugs fibrates and niacin and the triglyceride lowering/HDL raising PPAR alpha activators. There is a need to identify new targets for atherosclerosis therapy.

There have been significant advances made in understanding the role that inflammation plays in the process of atherosclerosis. Atherosclerosis involves many cell types and molecular factors (described in, for example, Ross (1993) Nature 362: 801-809). The process, in normal circumstances a protective response to insults to the endothelium and smooth muscle cells (SMCs) of the wall of the artery, consists of the formation of fibrofatty and fibrous lesions or plaques, preceded and accompanied by inflammation. The advanced lesions of atherosclerosis may occlude the artery concerned, and result from an excessive inflammatory-fibroproliferative response to numerous different forms of insult. Injury or dysfunction of the vascular endothelium is a common feature of many conditions that predispose an individual to accelerated development of atherosclerotic cardiovascular disease. There has been considerable effort in establishing that hypertension contributes to atherosclerosis. The identification of molecules that regulate blood pressure and vascular tone will be useful in discovering new therapies to treat cardiovascular diseases such as atherosclerosis.

### DETAILED DESCRIPTION OF THE INVENTION

The present invention provides methods and compositions for the diagnosis and treatment of cardiovascular disease. As used herein, disorders involving the heart, or “cardiovascular disease” or a “cardiovascular disorder” include a disease or disorder which affects the cardiovascular system, e.g., the heart, the blood vessels, and/or the blood. A cardiovascular disorder can be caused by an imbalance in arterial pressure, a malfunction of the heart, or an occlusion of a blood vessel, e.g., by a thrombus. A cardiovascular disorder includes, but is not limited to disorders such as arteriosclerosis, atherosclerosis, cardiac hypertrophy, ischemia reperfusion injury, restenosis, arterial inflammation, vascular wall remodeling, ventricular remodeling, rapid ventricular pacing, coronary microembolism, tachycardia, bradycardia, pressure overload, aortic bending, coronary artery ligation, vascular heart disease, valvular disease, including but not limited to, valvular degeneration caused by calcification, rheumatic heart disease, endocarditis, or complications of artificial valves; atrial fibrillation, long-QT syndrome, congestive heart failure, sinus node dysfunction, angina, heart failure, hypertension, high blood pressure, atrial fibrillation, atrial flutter, pericardial disease, including but not limited to, pericardial effusion and pericarditis; cardiomyopathies, e.g., dilated cardiomyopathy or idiopathic cardiomyopathy, myocardial infarction, coronary artery disease, coronary artery spasm, ischemic disease, arrhythmia, sudden cardiac death, cardiovascular disorders caused by lipoprotein metabolism disorders, including but not limited to, hyperlipidemia, dyslipidemia, hypercholesterolemia, hypertriglyceridemia and hyperlipoproteinemia; and cardiovascular developmental disorders (e.g., arteriovenous malformations, arteriovenous fistulae, raynaud's syndrome, neurogenic thoracic outlet syndrome, causalgia/reflex sympathetic dystrophy, hemangioma, aneurysm, cavernous angioma, aortic valve stenosis, atrial septal defects, atrioventricular canal, coarctation of the aorta, ebsteins anomaly, hypoplastic left heart syndrome, interruption of the aortic arch, mitral valve prolapse, ductus arteriosus, patent foramen ovale, partial anomalous pulmonary venous return, pulmonary atresia with ventricular septal defect, pulmonary atresia without ventricular septal defect, persistance of the fetal circulation, pulmonary valve stenosis, single ventricle, total anomalous pulmonary venous return, transposition of the great vessels, tricuspid atresia, truncus arteriosus, ventricular septal defects). A cardiovascular disease or disorder also can include an endothelial cell disorder.

As used herein, an “endothelial cell disorder” includes a disorder characterized by aberrant, unregulated, or unwanted endothelial cell activity, e.g., proliferation, migration, angiogenesis, or vascularization; or aberrant expression of cell surface adhesion molecules or genes associated with angiogenesis, e.g., TIE-2, FLT and FLK. Endothelial cell disorders include tumorigenesis, tumor metastasis, psoriasis, diabetic retinopathy, endometriosis, Grave's disease, ischemic disease (e.g., atherosclerosis), and chronic inflammatory diseases (e.g., rheumatoid arthritis).

A cardiovascular disease can also include thrombosis. Thrombosis can result from platelet dysfunction, e.g. seen in myocardial infarction, angina, hypertension, lipid disorders, diabetes mellitus; myelodysplastic syndromes; myeloproliferative syndromes (including polycythemia vera and thombocythemia); thrombotic thrombocytopenic purpuras; HIV-induced platelet disorders (AIDS-Thrombocytopenia); heparin induced thrombocytopenia; mural cell alterations/interactions leading to platelet aggregation/degranulation, vascular endothelial cell activation/injury, monocyte/macrophage extravasation and smooth muscle cell proliferation; autoimmune disorders such as, but not limited to vasculitis, antiphospholipid syndromes, systemic lupus erythromatosis; inflammatory diseases, such as, but not limited to Immune activation; graft vs. host disease; radiation induced hypercoagulation; clotting factor dysregulation either hereditary (autosomal dominant or recessive) such as, but not limited to clotting factor pathways including protein C/S, Anti-thrombin III deficiency, and the Factor V Leiden mutation or acquired such as but not limited to autoimmune, cancer -associated and drug-induced dysregulation of clotting factors.

“Treatment”, as used herein, is defined as the application or administration of a therapeutic agent to a patient, or application or administration of a therapeutic agent to an isolated tissue or cell line from a patient, who has a disease or disorder, a symptom of disease or disorder or a predisposition toward a disease or disorder, with the purpose of curing, healing, alleviating, relieving, altering, remedying, ameliorating, improving or affecting the disease or disorder, at least one symptom of disease or disorder or the predisposition toward a disease or disorder. A therapeutic agent includes, but is not limited to, small molecules, peptides, antibodies, ribozymes and antisense oligonucleotides. Representative molecules are described herein.

The present invention is based, at least in part, on the discovery that nucleic acid and protein molecules, (described infra), are differentially expressed in cardiovascular disease states relative to their expression in normal, or non-cardiovascular disease states. The modulators of the molecules of the present invention, identified according to the methods of the invention can be used to modulate (e.g., inhibit, treat, or prevent) or diagnose cardiovascular disease, including, but not limited to, atherosclerosis and thrombosis.

“Differential expression”, as used herein, includes both quantitative as well as qualitative differences in the temporal and/or tissue expression pattern of a gene. Thus, a differentially expressed gene may have its expression activated or inactivated in normal versus cardiovascular disease conditions (for example, in an experimental cardiovascular disease system such as in an animal model for atherosclerosis). The degree to which expression differs in normal versus cardiovascular disease or control versus experimental states need only be large enough to be visualized via standard characterization techniques, e.g., quantitative PCR, Northern analysis, subtractive hybridization. The expression pattern of a differentially expressed gene may be used as part of a prognostic or diagnostic cardiovascular disease, e.g., artherosclerosis and/or thrombosis, evaluation, or may be used in methods for identifying compounds useful for the treatment of cardiovascular disease, e.g., atherosclerosis and/or thrombosis. In addition, a differentially expressed gene involved in cardiovascular disease may represent a target gene such that modulation of the level of target gene expression or of target gene product activity will act to cure, heal, alleviate, relieve, alter, remedy, ameliorate, improve or affect a cardiovascular disease condition, e.g., atherosclerosis and/or thrombosis. Compounds that modulate target gene expression or activity of the target gene product can be used in the treatment of cardiovascular disease. Although the genes described herein may be differentially expressed with respect to cardiovascular disease, and/or their products may interact with gene products important to cardiovascular disease, the genes may also be involved in mechanisms important to additional cardiovascular cell processes.

Molecules of the Present Invention

Gene ID 1722

The human 1722 sequence (SEQ ID NO:1), known also as a serine/threonine protein kinase (Protein Kinase C mu or PKD), is approximately 3742 nucleotides long including untranslated regions. The coding sequence, located at about nucleic acids 236 to 2974 in SEQ ID NO:1, encodes a 912 amino acid protein (SEQ ID NO:2).

As determined by TaqMan analysis, 1722 mRNA was expressed in a variety of human tissues, including bladder, kidney, ovary, pituitary gland and veins. Additionally, 1722 mRNA was expressed in cultured smooth muscle cells, endothelial cells and normal human arteries. In rats treated with antihypertensive agents for three days, expression of 1722 mRNA was upregulated in aortas compared to vehicle treated controls. In spontaneously hypertensive rats, there was a significant upregulation (3 fold) of 1722 mRNA in aortas of the stroke-prone strain at 15 weeks of age compared to Wistar Kyoto (WKY) normotensive age-matched controls. In renin transgenic rats, there was downregulation of 1722 mRNA in aortas compared to wildtype Sprague-Dawley controls at 5 and 15 weeks of age.

1722 is a serine/threonine protein kinase known as Protein Kinase C mu or PKD (Zugaza et al., J Biol Chem, 272 (8), 23952-23960). It is a phorbol-ester/DAG-stimulated protein kinase. Factors known to be involved in vascular signaling, including angiotensin, endothelin, and vasopressin activate 1722 and cause downstream activation of other kinases, such as JNK and MAPK. Resveratrol, an antioxidant, has been shown to inhibit the autophosphorylation of 1722 (Biochem Pharmacol. 2001 Dec. 15;62(12):1647-51.) Resveratrol is the substance found in red wine which shows a beneficial effect in cardiovascular disease. Our expression and regulation data indicate that inhibition of 1722 kinase will result in a beneficial effect by reducing well known vasoconstrictor signaling cascades. The expression of 1722 mRNA is enhanced in hypertensive states and is expressed in relevant tissues involved in blood pressure maintenance (veins, arteries, kidney and skeletal muscle). Due to 1722 mRNA expression in smooth muscle cells, endothelial cells and normal human arteries, along with its functional role, modulators of 1722 activity would be useful in treating disorders associated with cardiovascular disease, including but not limited to atherosclerosis. 1722 polypeptides of the present invention are useful in screening for modulators of 1722 activity.

Gene ID 10280

The human 10280 sequence (SEQ ID NO:3), known also as protein L-isoaspartyl methyltransferase (PIMT), is approximately 1599 nucleotides long including untranslated regions. The coding sequence, located at about nucleic acid 74 to 757 of SEQ ID NO:3, encodes a 227 amino acid protein (SEQ ID NO:4).

As determined by TaqMan analysis, 10280 mRNA was expressed at high levels in brain, in megakaryocytes generated in vitro and in erythroid cells generated in vitro. 10280 mRNA was also expressed in heart. In addition, 10280 mRNA was detected at high levels in the platelets of patients with coronary artery disease and in platelets from normal volunteers.

Protein L-isoaspartyl methyltransferase or PIMT (10280), catalyzes the carboxyl methylation of isoaspartyl sites on deamidated asparaginyl residues [PNAS.1987. 84(9):2595-2599]. This activity is suggested to play a role in protein repair/degradation [PNAS. 1997. 94;6132-6137]. Calmodulin, an important mediator of platelet activation, is a target for PIMT, and studies demonstrate that inhibition of PIMT increases the degradation of calmodulin [JBC. 2002. 277(32); 28972-28980; JBC.1998. 273(4);28516-28523]. Based on the literature and our expression data, inhibition of 10280 and the subsequent degradation of Calmodulin would create platelets that were less reactive to prothrombotic stimuli. Therefore, inhibition of 10280 would provide a means to inhibit platelet reactivity and thrombus formation. Due to 10280 mRNA expression in the brain, in megakaryocytes generated in vitro and in erythroid cells generated in vitro, along with its functional role, modulators of 10280 activity would be useful in treating disorders associated with cardiovascular disease, including but not limited to thrombosis and atherosclerosis. 10280 polypeptides of the present invention are useful in screening for modulators of 10280 activity.

Gene ID 59917

The human 59917 sequence (SEQ ID NO:5), known also as a choline transporter-like protein, is approximately 4301 nucleotides long including untranslated regions. The coding sequence, located at about nucleic acid 43 to 2016 of SEQ ID NO:5, encodes a 657 amino acid protein (SEQ ID NO:6).

As determined by TaqMan analysis, 59917 mRNA was expressed at very high levels in megakaryocytes generated in vitro and in brain. 59917 mRNA was also expressed in endothelial cells. 59917 mRNA was detected in the platelets of patients with coronary artery disease and at lower levels in platelets from normal volunteers.

59917 or choline transporter-like protein is known to be expressed in dendritic cells [JI. 2001. 167:5795-5804]. Choline transport is presumed to be an important regulatory step in the formation of phosphatidylcholine, an important component of the plasma membrane. Therefore, choline uptake is required for platelet reactivity. Activated platelets release biologically active phospholipids into the plasma where they negatively affect hemostasis [JBC.2002. 277(50):48737-48744]. Based on the literature and our expression data, inhibition of 59917 would decrease platelet reactivity and inhibit thrombus formation. Due to 59917 mRNA expression in megakaryocytes generated in vitro and in brain, along with its functional role, modulators of 59917 activity would be useful in treating disorders associated with cardiovascular disease including but not limited to thrombosis and atherosclerosis. 59917 polypeptides of the present invention are useful in screening for modulators of 59917 activity.

Gene ID 85553

The human 85553 sequence (SEQ ID NO:7), known also as a carbonic anhydrase, is approximately 3564 nucleotides long including untranslated regions. The coding sequence, located at about nucleic acid 52 to 840 of SEQ ID NO:7, encodes a 262 amino acid protein (SEQ ID NO:8).

As assessed by TaqMan analysis, 85553 mRNA was expressed at high levels in megakaryocytes generated in vitro and in the brain. 85553 mRNA was also expressed at lower levels in kidney, small intestine and colon. 85553 RNA was detected in the platelets of patients with coronary artery disease and in platelets from normal volunteers.

85553 is 60% identical to human carbonic anhydrase. Inhibition of human carbonic anhydrase results in decreased platelet aggregation [BBRC. 1984.121(1):266-70.]. 85553 contains the catalytic domain of carbonic anhydrase indicating that it functions similarly to human carbonic anhydrase. The highly restricted expression profile of 85553 mRNA, together with its functional domains suggest that inhibition of 85553 would decrease platelet reactivity and inhibit thrombus formation. Due to 85553 mRNA expression in megakaryocytes generated in vitro and in brain, along with its functional role, modulators of 85553 activity would be useful in treating disorders associated with cardiovascular disease including but not limited to thrombosis and atherosclerosis. 85553 polypeptides of the present invention are useful in screening for modulators of 85553 activity.

Gene ID 10653

The human 10653 sequence (SEQ ID NO:9), known also as Transketolase (EC 2.2.1.1), is approximately 2106 nucleotides long including untranslated regions. The coding sequence, located at about nucleic acid 71 to 1942 of SEQ ID NO:9, encodes a 623 amino acid protein (SEQ ID NO: 10).

As determined by TaqMan analysis, 10653 mRNA was downregulated by cholesterol in the livers of African Green Monkeys (1.5 fold, p=0.05). This correlates with the downregulation of the control gene HMGCoA Synthase which is involved in cholesterol synthesis. 10653 mRNA was also downregulated in liver by two and six months of cholesterol feeding of the Apolipoprotein E (ApoE) knockout mice (1.3 fold). 10653 mRNA was also downregualted in the livers of marmosets treated with lipid-lowering therapies including niacin, probucol, cerivastatin, fenofibrate, cholestyramine, and gemfibrozil.

10653 is a transketolase (TKT), which is an enzyme in the pentose-phosphate pathway and it is most active in the liver (Mol Cell Biochem, 1999, 201:57-63). Transketolase serves as a reversible link between the hexose monophosphate shunt and the glycolytic pathway to regulate NADPH and ribose-5-phosphate synthesis. NADPH is known to be important for fatty acid and steroid synthesis. Transketolase haploinsufficent mice have reduced body fat and reduced liver mass (Mol Cell Biol. 2002, 22:6142-6147). TKT +/− mice had normal growth hormone levels but reduced leptin levels.

The downregulation of 10653 mRNA expression in the liver by cholesterol feeding is consistent with 10653 playing a role in production of NADPH for cholesterol and triglyceride synthesis for VLDL production. In addition, 10653 regulation mimics that of ApoCII indicating that 10653 plays a role in the generation and/or metabolism of triglyceride-rich particles. Inhibition of 10653 reduces VLDL synthesis by the liver and thereby reduces plasma VLDL cholesterol, LDL cholesterol and triglycerides. Due to 10653 mRNA expression in the liver, along with its functional role, modulators of 10653 activity would be useful in treating disorders associated with cardiovascular disease, including but not limited to heart faliure. 10653 polypeptides of the present invention are useful in screening for modulators of 10653 activity.

Gene ID 9235

The human 9235 sequence (SEQ ID NO: 11), known also as Glucosamine-Fructose-6-phosphate Amido Transferase (GFAT), is approximately 3082 nucleotides long including untranslated regions. The coding sequence, located at about nucleic acid 123 to 2168 of SEQ ID NO:11, encodes a 681 amino acid protein (SEQ ID NO:12).

As determined by TaqMan analysis, 9235 mRNA was downregulated by cholesterol in the livers of African Green Monkeys (1.3 fold, p=0.09). This regulation mimics that of HMGCoA reductase, a gene involved in cholesterol synthesis. 9325 mRNA was also downregulated in liver by two and six months of cholesterol feeding of the Apolipoprotein E (ApoE) knockout mice (1.8 fold). This correlates with the downregulation of the control gene HMGCoA Synthase which is involved in cholesterol synthesis. 9325 mRNA was upregulated in the livers of marmosets treated with niacin (1.3 fold), and cholestyramine (1.4 fold).

Inhibition of 9325 potentially results in reduced activation of the hexosamine pathway and therefore reduced plasma LDL-C and triglyceride levels. Inhibition of 9325 also results in the protection against glycation-mediated LDL uptake and foam cell formation in the vascular wall of hyperlipidemics. Due to 9325 mRNA expression in the liver, along with its functional role, modulators of 9325 activity would be useful in treating disorders associated with cardiovascular disease including but not limited to heart failure. 9325 polypeptides of the present invention are useful in screening for modulators of 9325 activity.

Gene ID 21668

The human 21668 sequence (SEQ ID NO:13), known also as a 17 beta-hydroxysteroid dehydrogenase type 7 (17HSD7), is approximately 1453 nucleotides long including untranslated regions. The coding sequence, located at about nucleic acid 12 to 1037 of SEQ ID NO:13, encodes a 341 amino acid protein (SEQ ID NO:14).

As assessed by TaqMan analysis, 21668 mRNA was downregulated in paired liver samples of Wake Forest monkeys fed with and without a high cholesterol diet (6 pairs). 21668 mRNA was found to be down 0.71-fold due to a cholesterol diet. In marmoset models treated with cerivastatin and cholestyramine (cholesterol lowering agents), there was a marked increase in 21668 mRNA expression. HMG-CoA synthase was also highly increased in cerivastatin treated monkeys.

The human 17HSD7 has been mapped to chromosome 10p11.2 which is close to susceptibility loci for hyperlipidemia, obesity and diabetes. Reported studies on comparative phylogenetic analysis and molecular modeling suggest a role for 21668 or 17HSD7 in cholesterogenesis. 21668 or 17HSD7 shows significant homology to the yeast 3-ketosteroid reductase, ERG27, involved in ergosterol biosynthesis (cholesterol equivalent in yeast) (Breitling et al. Mol. Cell. Endocrinol. 2001 Jan. 22; 171 (1-2): 199-204). ERG27 converts zymosterone to zymosterol (precursor to cholesterol and ergosterol).

17HSD Type 1, 3 and 7 are responsible for catalyzing estrone to estradiol, type 1 being the main enzyme. However, it is thought that type 7 potentially plays an important role in other pathways in addition to the inactivation of dihydrotestosterone. Since 21668 or 17HSD7 is expressed in the liver (major site of cholesterol synthesis), since its expression pattern in various microarray profiling experiments is similar to other cholesterol synthesis enzymes and since it is significantly homologous to ERG27, 17HSD7 is potentially involved in an alternative pathway to cholesterol synthesis where it converts a cholesterol ketone precursor to the corresponding alcohol. Due to 21668 mRNA expression in the liver, along with its functional role, modulators of 21688 activity would be useful in treating disorders associated with cardiovascular disease including but not limited to heart failure. 21688 polypeptides of the present invention are useful in screening for modulators of 21688 activity.

Gene ID 17794

The human 17794 sequence (SEQ ID NO:15), known also as Pyruvate Dehydrogenase Kinase 3 (PDK3), is approximately 1599 nucleotides long including untranslated regions. The coding sequence, located at about nucleic acid 23 to 1243 of SEQ ID NO:15, encodes a 406 amino acid protein (SEQ ID NO:16).

As assessed by TaqMan analysis, 17794 mRNA has a fairly restricted expression profile in human tissues, including high expression in arteries, veins, CNS structures, ovary and pituitary gland. Additionally, 17794 mRNA was also expressed in cultured smooth muscle cells and endothelial cells. There was a significant (t-test, p<0.05) upregulation of 17794 mRNA in human failing hearts compared to non-failing controls.

17794 is known as pyruvate dehydrogenase kinase 3 (PDK3) and it is a mitochondrial isoform of the enzyme (J Bio Chem, Baker et al., 275; 15773-15781, 2000). The 17794 protein acts to inhibit the pyruvate dehydrogenase complex, which converts pyruvate to acetyl CoA. A non-specific inhibitor of PDK isoforms, dichloroacetate (DCA) has been shown to improve cardiac function in both rats and humans (Am J Physiol Heart Circ Physiol, Taniguchi et al., 280; H1762-1769, 2001 and Clin Sci (Lond), Ludvik et al., 80; 47-51, 1991). Specifically, DCA reduced peripheral vascular resistance after two intravenous bolus doses in humans. In patients with pulmonary arterial hypertension, oral DCA promoted a more oxidized redox state by restoring voltage-gated potassium channel expression (Kv) (Adv Exp Med Biol, Michelakis et al., 502; 401-418, 2001). Inhibiting 17794 with a more specific and potent inhibitor than DCA would produce a vessel-enriched increase in K+ currents resulting in decreased Ca2+ influx and vasodilation. In addition, an improvement in cardiac function would result in failing and ischemic myocardium due to increased glucose oxidation and ATP utilization. Due to 17794 mRNA expression in arteries, veins, CNS structures, ovary and pituitary gland, along with its functional role, modulators of 17794 activity would be useful in treating disorders associated with cardiovascular disease including but not limited to heart failiure and atherosclerosis. 17794 polypeptides of the present invention are useful in screening for modulators of 17794 activity.

Gene ID 2210

The human 2210 sequence (SEQ ID NO:17), known also as CaMKK Beta 1, is approximately 1767 nucleotides long. The coding sequence, located at about nucleic acid 1 to 1767 of SEQ ID NO:17, encodes a 588 amino acid protein (SEQ ID NO:18).

As assessed by TaqMan analysis, 2210 mRNA was expressed in human arteries, veins, HUVECS, skeletal muscle and brain cortex. Further TaqMan analysese indicated that 2210 mRNA was upregulated in spontaneously hypertensive stroke prone (SHR SP) rat aortas at an old age compared to SHR and normotensive Wistar Kyoto (WKY). 2210 mRNA was also upregulated in aortas of rats treated with antihypertensive agents for 3 days. Lastly, 2210 mRNA was upregulated in failing human hearts compared to non-failing hearts as well as in mouse models of heart failure (isoproterenol infusion at old age, aortic banding/pressure overload).

Inhibition of CaMKK beta 1 will have a beneficial effect on vasculartone and heart disease via the reduction of CaMKI and CaMKIV activity and the blockade of downstream signaling pathways of these kinases. Known targets for CaMK's are transcription factors (ex. CREB) which stimulate the production of such targets as aldosterone synthase (RAS pathway) and CD40L in ECs (relevant to the pathogenesis of atherosclerosis and myocardial infarction) as well as cellular proteins which modulate calcium handling (L-type Ca channel, ryanodine receptor, etc.). CaMKI and CaMKIV induce hypertrophic responses in cardiomyocytes in vitro. CaMKIV overexpressing mice develop cardiac hypertrophy with increased left ventricular end-diastolic diameter and decreased fractional shortening. CaMKIV activates the transcription factor MEF2 through a posttranslational mechanism in the hypertrophic heart in vivo. Due to 2210 mRNA expression in human arteries, veins, HUVECS, skeletal muscle and brain cortex, along with its functional role, modulators of 2210 activity would be useful in treating disorders associated with cardiovascular disease including but not limited to vasculartone and heart failure. 2210 polypeptides of the present invention are useful in screening for modulators of 2210 activity.

Gene ID 6169

The human 6169 sequence (SEQ ID NO:19), known also as human ER25, is approximately 1465 nucleotides long including untranslated regions. The coding sequence, located at about nucleic acid 91 to 972 of SEQ ID NO:19, encodes a 293 amino acid protein (SEQ ID NO:20).

As determined by TaqMan analysis, 6169 mRNA was expressed in the following tissues: brain cortex, liver (fibrosis), erythroid cells, liver, kidney and dorsal root ganglion. 6169 mRNA was also downregulated in the liver by cholesterol feeding in African Green Monkeys (1.3 fold, p=0.02) and by polyunsaturated fatty acids in the diet of African Green Monkeys (1.8 fold, p=0.001). 6169 mRNA was also upregulated by Mevastatin treatment in human primary hepatocytes (2.4 fold) and by Cerivastatin in Marmoset livers (2.4 fold).

6169 has not been given an enzymatic classification number. Enzymatic activity of 6169 has not been demonstrated, but it has been suggested based on its 38% identity to the yeast methyl sterol oxidase (ERG25). Data in the literature indicates that a mammalian sterol methyl oxidase exists, but this activity has not been linked to 6169. Our data provides a link between 6169 and mammalian lipid metabolism. Experiments on several models of lipid metabolism in monkeys and man have demonstrated that 6169 gene expression responds both to lipid feeding (cholesterol and polyunsaturated fatty acids) and to cholesterol reduction by chemical inhibition (Mevastatin and Cerivastatin). Together this data indicates that 6169 plays a role in lipid metabolism in primates. Given its homology to yeast methyl sterol oxidase, 6169 potentially plays a direct role in cholesterol metabolism and in the generation of oxysterols which regulate the expression of genes involved in lipid metabolism through FXR and LXR. Due to 6169 mRNA expression in several models of lipid metabolism, along with its functional role, modulators of 6169 activity would be useful in treating disorders associated with cardiovascular disease, including but not limited to atherosclerosis. 6169 polypeptides of the present invention are useful in screening for modulators of 6169 activity.

Gene ID 10102

The human 10102 sequence (SEQ ID NO:21), known also as human Myo-inositol-1(or 4)-monophosphatase (EC 3.1.3.25) (IMPase) (IMP) (Inositol monophosphatase) (Lithium-sensitive myo-inositol monophosphatase A1), is approximately 897 nucleotides long including untranslated regions. The coding sequence, located at about nucleic acid 37 to 870 of SEQ ID NO:21, encodes a 277 amino acid protein (SEQ ID NO:22).

As determined by TaqMan analysis using a human tissue panel, 10102 mRNA was fairly ubiquitously expressed in all of the 40 tissues and cells tested. 10102 mRNA was significantly upregulated by many lipid lowering drugs including Niacin, Cholestyramine, Gemfibrozil, Probucol, and Cerivastatin.

Enzymatic activity of 10102 has been demonstrated to play a role in generating inositol required for the synthesis of phosphatidylinositol and polyphosphoinositides. The regulation of 10102 by lipid-lowering agents indicates that 10102 may be upregulated to counter the reduction of cholesterol/lipid levels. Since 10102 is known to play a role in signaling, 10102 activity potentially plays a role in the sensing and signaling of lipid availability to cells. Inhibition of 10102 would lead to a reduction in cellular synthesis of lipids and would therefore provide a treatment for excess lipids which are deleterious in atherosclerotic disease. Due to 10102 mRNA upregulation by lipid-lowering agents, along with its functional role, modulators of 10102 activity would be useful in treating disorders associated with cardiovascular disease, including but not limited to atherosclerosis. 10102 polypeptides of the present invention are useful in screening for modulators of 10102 activity.

Gene ID 21061

The human 21061 sequence (SEQ ID NO:23), known also as homo sapiens channel kinase 1 (CHAK1, LTRPC7 or TRP-PLIK), is approximately 7280 nucleotides long including untranslated regions. The coding sequence, located at about nucleic acid 214 to 5871 of SEQ ID NO:23, encodes a 1885 amino acid protein (SEQ ID NO:24).

As determined by TaqMan analysis, 21061 mRNA was predominantly expressed in highly vascularized tissues such as heart, skeletal muscle, liver and kidney. 21061 mRNA was highly expressed in human vascular cells such as coronary smooth muscle cells and umbilical vein endothelial cells. There is a trend for higher expression in arteries compared to veins. There is high expression of 21061 mRNA in CNS structures, including cortex and spinal cord. In rat aortas, 21061 mRNA was expressed at high levels and was regulated in various vascular tone models. Specifically, 21061 mRNA was down-regulated in transgenic renin rat aortas compared to wild-types and was up-regulated with angiotensin-1 receptor blockade in normotensive rats.

21061 is a human channel kinase 1 (CHAK1, LTRPC7 or TRP-PLIK) which belongs to the TRPM (melastatin-like) family. Channel kinases contain a novel alpha kinase (EF2 kinase) domain and 21061 regulates channel activity by autophosphorylation (Runnels 2001 Science 291 1043; Nadler et al., Nature 411 590-595). Based on its annotation, 21061 is likely to function in the action of calcium-mediated cellular responses. Given the well characterized role of calcium signaling in vascular reactivity and the expression pattern of 21061 mRNA, inhibition of 21061 would reduce intracellular calcium levels and promote vasodilation and blood pressure lowering. Due to 21061 mRNA expression in highly vascularized tissues, along with its functional role, modulators of 21061 activity would be useful in treating disorders associated with cardiovascular disease, including but not limited to atherosclerosis. 21061 polypeptides of the present invention are useful in screening for modulators of 21061 activity.

Gene ID 17662

The human 17662 sequence (SEQ ID NO:25), known also as vitellogenic carboxypeptidase-like protein (CPVL), is approximately 1638 nucleotides long including untranslated regions. The coding sequence, located at about nucleic acid 70 to 1500 of SEQ ID NO:25, encodes a 476 amino acid protein (SEQ ID NO:26).

As determined by TaqMan analysis, 17662 mRNA was expressed in a variety of human tissues, primarily in vascularized tissues such as the artery, vein, heart, kidney and skeletal muscle. Additionally, 17662 mRNA was expressed in mononuclear cells, spleen, and erythroid progenitor cells. 17662 mRNA was also significant upregulated in atherosclerotic human arteries compared to contro samples (t-test, p<0.01). In cultured human monocytes and macrophages, 17662 mRNA was robustly expressed. There was also upregulation of 17662 mRNA in failing human ventricles compared to non-failing hearts.

17662 is a serine carboxypeptidase known as vitellogenic carboxypeptidase-like protein (CPVL) (Mahoney JA et al., Genomics 2001) which was cloned from human macrophages. 17662 was shown to be induced during maturation of monocytes into macrophages. Our data indicate that overexpression of 17662 mRNA is associated with severity of human vessel disease. A potential role for 17662 includes participation in an inflammatory cascade in coronary artery disease and diminution of atherosclerosis progression. Due to 17662 mRNA expression in vascularized tissues, along with its functional role, modulators of 17662 activity would be useful in treating disorders associated with cardiovascular disease, including but not limited to atherosclerosis. 17662 polypeptides of the present invention are useful in screening for modulators of 17662 activity.

Gene ID 1468

The human 1468 sequence (SEQ ID NO:27), known also as Hematopoietic Cell Kinase (HCK), is approximately 2015 nucleotides long including untranslated regions. The coding sequence, located at about nucleic acid 169 to 1686 of SEQ ID NO:27, encodes a 505 amino acid protein (SEQ ID NO:28).

As determined by TaqMan analysis, 1468 mRNA was expressed in a variety of human tissues, including heart, spleen, tonsil, lung, skeletal muscle and hypothalamus. Additionally, 1468 mRNA was expressed in macrophages, neutrophils, and erythroid progenitor cells. 1468 mRNA was also significantly upregulated in atherosclerotic human arteries compared to normal tissues (t-test, p<0.05). In cultured human monocytes and macrophages, 1468 mRNA was both highly expressed as well as regulated by lipid-loading with oxidized LDL and deloading with apolipoprotein A1. This expression is highly correlated with IL1-beta levels, indicating that the 1468 was regulated in a similar way to an inflammatory marker. In cultured human monocytes and macrophages, the 1468 mRNA was upregulated by 18 hours of CD40 ligand stimulation.

1468 is a member of the src family of tyrosine kinases and is referred to as Hematopoietic Cell Kinase (HCK)(Scott, MP, et al., 2002, J Biol Chem, 277: 28238-28246). 1468 has been described as playing a role in phagocytosis and in receptor-mediated signaling in monocytes. Some specific substrates for 1468 include Wiskott-Aldrich syndrome protein (WASP), WASP-interacting protein(WIP)(Scott, MP, et al., 2002, J Biol Chem, 277: 28238-28246), and VAV (English BK et al., 1997, J Leukoc Biol, 62(6):859-64). These substrates are known regulators of the actin cytoskeleton and are activated by inflammatory stimuli. Stimulation of 1468 kinase by sE-selectin promotes monocyte chemotaxis (Kumar, P. et al., 2001, J Biol Chem, 276; 21039-21045). The literature strongly indicates a role for 1468 in monocyte and macrophage activites known to play a deleterious role in blood vessel disease. Therefore, inhibition of 1468 would result in a reduction of monocyte recruitment into atherosclerotic plaques and a diminished lesion size. Due to 1468 mRNA expression in a variety of vascular tissues, along with its functional role, modulators of 1468 activity would be useful in treating disorders associated with cardiovascular disease, including but not limited to atherosclerosis. 1468 polypeptides of the present invention are useful in screening for modulators of 1468 activity.

Gene ID 12282

The human 12282 sequence (SEQ ID NO:29), known also as Toll-Like Receptor 8 (TLR8), is approximately 3367 nucleotides long including untranslated regions. The coding sequence, located at about nucleic acid 152 to 3331 of SEQ ID NO:29, encodes a 1059 amino acid protein (SEQ ID NO:30).

As determined by TaqMan analysis, 12282 mRNA expression was observed in macrophages, PBMC cells, lung, spleen, ovary, synovium and the lymph node. 12282 mRNA was also upregulated in atherosclerotic human arteries relative to normal tissues (t-test, p<0.05). In cultured human monocytes, 12282 mRNA is both highly expressed as well as regulated by lipid-loading with oxidized LDL and deloading with apolipoprotein A1; this expression is highly correlated with IL1-beta levels, suggesting that 12282 is regulated in a similar way to an inflammatory marker. 12282 mRNA was also upregulated in cultured human monocytes and macrophages after 18 hours of interferon gamma stimulation. In the ApoE knockout model, 12282 mRNA was upregulated in aortic arches when compared to the less diseased abdominal aortic segments in 20 and 26 week old animals.

12282 is known as Toll-Like Receptor 8 (TLR8). Toll-like receptors are known to mediate innate immunity via the activation of a variety of transcriptional events. TLR8 is unlike many of the earlier characterized TLR's in that it has not previously been shown to be expressed in atherosclerosis. TLR's act on many transcription factors which are known to be induced in inflammation, such as NF-kB, STAT and AP-1 (Lee, JY et al., 2003, J Lipid Res, 44: 479-486). Inhibition of these receptors by n-3 polyunsaturated fatty acids is shown to be beneficial and reduce cyclooxygenase 2 expression (Lee, JY et al., 2003, J Lipid Res, 44: 479486). Therefore, specific regulation of 12282 by oxidized lipids and its expression pattern in diseased vessels, identify 12282 as a new target of inhibition for atherosclerosis. Due to 12282 mRNA expression in human arteries, along with its functional role, modulators of 12282 activity would be useful in treating disorders associated with cardiovascular disease, including but not limited to atherosclerosis. 12282 polypeptides of the present invention are useful in screening for modulators of 12282 activity.

Gene ID 6350

The human 6350 sequence (SEQ ID NO:31), known also as carboxypeptidase M, is approximately 2085 nucleotides long including untranslated regions. The coding sequence, located at about nucleic acid 76 to 1407 of SEQ ID NO:31, encodes a 443 amino acid protein (SEQ ID NO:32).

As determined by TaqMan analysis, 6350 mRNA was expressed in a wide range of human tissues, including primarily kidney, adipose tissue, breast, lung with chronic obstructive pulmonary disease, hemangioma, brain cortex and hypothalamus. Additionally, 6350 mRNA was expressed in arteries and veins. 6350 mRNA was also highly significantly upregulated in atherosclerotic human arteries compared to normal tissues (t-test, p=0.0001). In cultured human monocytes, 6350 was regulated by lipid-loading with oxidized LDL and deloading with apolipoprotein Al. 6350 mRNA was also regulated in both monocytes and macrophages with moderately oxidized and acetylated LDL stimulation. 6350 mRNA was also upregulated in cultured human macrophages after 18 hours of CD40 ligand stimulation.

6350 is known as carboxypeptidase M, which is an arginine/lysine carboxypeptidase, that is membrane bound (Skidgel, RA et al., 1989, J Bio Chem, 264: 2236-2241). The enzymatic functions of 6350 include to either i) activate or ii) inactivate peptide hormones, such as anaphylatoxins and enkephalins. One specific substrate of 6350 is bradykinin. The action of carboxypeptidase M on bradykinin causes the formation of des-Arg9-bradykinin, which acts on the B 1 receptor subtype. Stimulation of the B 1 receptor is known to cause inflammatory responses and increased vascular tone. Therefore, inhibition of 6350 would result in a shift of the kinin reaction, toward the build-up of bradykinin, as well as action on the B2 receptor, which promotes vasodilation and nitric oxide formation, both of which can be beneficial in cardiovascular disease. Due to 6350 mRNA upregulation in human arteries, along with its functional role, modulators of 6350 activity would be useful in treating disorders associated with cardiovascular disease, including but not limited to vasodilation. 6350 polypeptides of the present invention are useful in screening for modulators of 6350 activity.

Gene ID 9035

The human 9035 sequence (SEQ ID NO:33), known also as CD38, is approximately 1408 nucleotides long including untranslated regions. The coding sequence, located at about nucleic acid 104 to 1006 of SEQ ID NO:33, encodes a 300 amino acid protein (SEQ ID NO:34).

As determined by TaqMan analysis, 9035 mRNA was expressed in a variety of human tissues, including skeletal muscle, prostate, tonsil, lymph node, spleen, brain cortex and hypothalamus. Additionally, 9035 mRNA was expressed in macrophages and neutrophils. 9035 mRNA was significantly upregulated in atherosclerotic human arteries compared to normal tissues (t-test, p<0.01). In cultured human monocytes, 9035 mRNA was both highly expressed as well as regulated by lipid-loading with oxidized LDL and deloading with apolipoprotein A1. 9035 mRNA was upregulated in cultured human monocytes and macrophages after 18 hours of interferon gamma stimulation. In the ApoE knockout model, 9035 mRNA was dramatically upregulated in aortic arches compared to the less diseased abdominal aortic segments.

9035 is known as CD38, which acts as a surface receptor and has enzymatic activity in the conversion of NAD+ to ADP ribose (Deaglio, S et al., 2001, Leuk Res, 25: 1-12). CD38 interacts weakly with its ligand, CD31 on endothelial cells, serving to promote cell-to-cell contact. The end product from the enzymatic reaction mediated by CD38, namely cyclic ADP ribose, is a well known second messenger which causes calcium release (Barone F, et al., 2002, FASEB Journal, 16: 697-705). Intracellular calcium surges result in a range of toxic effects, from activation of calcium dependent enzymes to expression of adhesion molecules. The enzymatic and signaling functions of CD38 are major aspects of the disease process in vessel wall atherosclerosis, from the earliest events of monocyte and lymphocyte adhesion to endothelial cells to the activation of monocytes and macrophages and the generation of toxic inflammatory molecules. Therefore, inhibition of 9035 would result in reduction of monocyte adhesion and extravasation into damaged vascular endothelium and diminished calcium flux in macrophages and monocytes. Due to 9035 mRNA expression in highly vascularized tissues, along with its functional role, modulators of 9035 activity would be useful in treating disorders associated with cardiovascular disease, including but not limited to atherosclerosis. 9035 polypeptides of the present invention are useful in screening for modulators of 9035 activity.

Gene ID 1820

The human 1820 sequence (SEQ ID NO:35), known also as mast cell chymase, is approximately 769 nucleotides long including untranslated regions. The coding sequence, located at about nucleic acid 16 to 759 of SEQ ID NO:35, encodes a 247 amino acid protein (SEQ ID NO:36).

As determined by TaqMan analysis, 1820 mRNA was expressed at highest levels in brain, in mast cells and in megakaryocytes generated in vitro. 1820 mRNA was detected in the platelets of patients with coronary artery disease and in platelets from normal volunteers.

Mast cell chymase has not been previously identified as a megakaryocyte/platelet product. The high levels of 1820 mRNA found in megakaryocytes and platelets indicate that 1820, a soluble granule protein, regulates clot formation following platelet degranulation. Therefore, inhibition of 1820 would provide a means to regulate platelet-rich thrombus formation Due to 1820 mRNA expression in the platelets of patients with coronary artery disease, along with its functional role, modulators of 1820 activity would be useful in treating disorders associated with cardiovascular disease, including but not limited to thrombosis. 1820 polypeptides of the present invention are useful in screening for modulators of 1820 activity.

Gene ID 23652

The human 23652 sequence (SEQ ID NO:37), known also as N-acetyltransferase-6 (NAT-6), is approximately 1272 nucleotides long including untranslated regions. The coding sequence, located at about nucleic acid 212 to 1072 of SEQ ID NO:37, encodes a 286 amino acid protein (SEQ ID NO:38).

As determined by TaqMan analysis, 23652 mRNA was expressed at high levels in the brain, megakaryocytes generated in vitro, and in endothelial cell cultures. 23652 mRNA was also expressed, but at lower levels in kidney and heart. 23652 mRNA was detected in the platelets of patients with coronary artery disease and in platelets from normal volunteers.

N-acetyltransferase 1 and 2 are implicated in lung cancer, however the function of NAT-6 or 23652 has not previously been ascertained. The high levels of 23652 mRNA found in megakaryocytes and platelets indicate that 23652 plays a role in post-translational modification of granule protein(s). Therefore, inhibition of 23652 would provide a means to inhibit platelet-granule mediated thrombus formation. Due to 23652 mRNA expression in platelets of patients with coronary artery disease, along with its functional role, modulators of 23652 activity would be useful in treating disorders associated with cardiovascular disease, including but not limited to thrombosis. 23652 polypeptides of the present invention are useful in screening for modulators of 23652 activity.

Gene ID 7301

The human 7301 sequence (SEQ ID NO:39), known also as Adomet synthetase, is approximately 3386 nucleotides long including untranslated regions. The coding sequence, located at about nucleic acid 223 to 1410 of SEQ ID NO:39, encodes a 395 amino acid protein (SEQ ID NO:40).

As determined by TaqMan analysis, 7301 mRNA was down-reregulated in the livers of marmosets treated with lipid-lowering therapies such as niacin, probucol, fenofibrate, gemfibrozil, and cerivastatin. 7301 mRNA was also down-regulated in the livers of mice during treatment with ciprofibrate.

7301 is Adomet synthetase, a protein that converts methionine and adenosine to Adomet. A reduction in 7301 and an increase in other known genes by lipid-lowering therapies cause a reduction in the levels of Adomet in the liver. Adomet is a metabolic sensor in the liver, thereby causing levels of Adomet signaling in the liver to either move towards a metabolic state or towards a repair state. When in the repair state, such as after liver injury by hepatectomy, the liver reduces the secretion of very low density lipoprotein (VLDL) particles. Therefore, the partial inhibition of 7301 would orient the liver towards a repair mode and would result in decrease of VLDL secretion. This would be beneficial for the treatment of hyperlipidemia and atherosclerosis. Due to 7301 mRNA expression in various lipid models, along with its functional role, modulators of 7301 activity would be useful in treating disorders associated with cardiovascular disease, including but not limited to atherosclerosis. 7301 polypeptides of the present invention are useful in screening for modulators of 7301 activity.

Gene ID 8925

The human 8925 sequence (SEQ ID NO:41), known also as Aldehyde oxidase (AO) (EC 1.2.3.1), is approximately 4170 nucleotides long including untranslated regions. The coding sequence, located at about nucleic acid 101 to 4117 of SEQ ID NO:41, encodes a 1338 amino acid protein (SEQ ID NO:42).

As determined by TaqMan analysis, 8925 mRNA was upregulated in the livers of marmosets treated with lipid-lowering therapies such as niacin, cholestyramine, and cerivastatin. 8925 mRNA was expressed only in the liver and adrenal glands of over 40 tissues and cells tested.

8925 is Aldehyde oxidase (AO) which belongs to the family molybdo-flavoenzymes (MFEs) that includes xanthine oxidase and recently the identified mouse aldehyde oxidase homologs 1, 2 and 3. Molybdenum acts as a biologically active cofactor (MoCo). 8925 or AO oxidizes aromatic aza-heterocycles containing a —CH═N— chemical function (phthalazine and purines), aromatic or non-aromatic charged aza-heterocycles (N1-methylnicotinamide and N-methylphtalazinium) or aldehydes such as benzaldehyde, retinal and vanillin. Given the upregulation of AO by lipid-lowering therapies, 8925 or AO potentially plays a role in the generation of a compound that stimulates VLDL synthesis. For example, AO can convert retinal to retinoic acid which is a ligand for nuclear hormone receptors such as RXR and RAR. These receptors form heterodimers with other nuclear hormone receptors such as LXR and PXR. The heterodimers that are formed can regulate the expression of genes involved in lipid synthesis and metabolism. Therefore, an inhibitor of 8925 will reduce the level of a lipid-related nuclear hormone in the liver and affect the expression of lipid-synthesis genes. Due to 8925 mRNA expression in various models of lipid metabolism, along with its functional role, modulators of 8925 activity would be useful in treating disorders associated with cardiovascular disease, including but not limited to atherosclerosis. 8925 polypeptides of the present invention are useful in screening for modulators of 8925 activity.

Gene ID 8701

The human 8701 sequence (SEQ ID NO:43), known also as alcohol dehydrogenase (ADHFe1), is approximately 1981 nucleotides long including untranslated regions. The coding sequence, located at about nucleic acid 15 to 1418 of SEQ ID NO:43, encodes a 467 amino acid protein (SEQ ID NO:44).

As determined by TaqMan analysis, 8701 mRNA was upregulated in the livers of marmosets treated with lipid-lowering therapies such as an MTP inhibitor, cholestyramine, gemfibrozil, fenofibrate, and cerivastatin.

8701 is an alcohol dehydrogenase (ADHFe1) which causes a reversible oxidation of alcohols to aldehydes or ketones using NAD(P) as a cofactor. It is well established that moderate alcohol intake increases HDL cholesterol and plasma ApoAI (e.g. J Lipid Res 2001, 42:2077-83: Alcohol Clin Exp Res 2002, 26: 1430-5). It has been shown that moderate drinkers who are homozygous for a slow-oxidizing form of another alcohol dehydrogenase (ADH3) have higher HDL levels and a substantially decreased risk of myocardial infarction (N Engl J Med 2001, 344:549-555). The mechanism by which this protection occurs is unknown. Because 8701 is highly regulated by lipid-lowering therapies, inhibition of 8701 should increase HDL, decrease LDL, and be protective for atherosclerosis. Due to 8701 mRNA expression in various models of lipid metabolism, along with its functional role, modulators of 8701 activity would be useful in treating disorders associated with cardiovascular disease, including but not limited to atherosclerosis. 8701 polypeptides of the present invention are useful in screening for modulators of 8701 activity.

Gene ID 3533

The human 3533 sequence (SEQ ID NO:45), known also as a serine protease or human reelin (RELN) (EC 3.4.21), is approximately 11580 nucleotides long including untranslated regions. The coding sequence, located at about nucleic acid 176 to 10558 of SEQ ID NO:45, encodes a 3460 amino acid protein (SEQ ID NO:46).

As determined by TaqMan analysis using a human tissue panel, 3533 mRNA was highly expressed in the dorsal root ganglion, spinal cord, brain, and liver out of over 40 tissues and cells tested. 3533 mRNA was significantly downregulated by many lipid lowering drugs including niacin, cholestyramine and statins. 3533 mRNA was also downregulated by T3 and variable regulation of 3533 was observed by fibrates.

Reelin or 3533 is a large secreted molecule with an active serine protease and it has been shown to be important in neuronal guidance in the developing nervous system. Reelin plays a role in layering of neurons in the cerebral cortex and cerebellum, it regulates microtubule function in neurons and neuronal migration and it affects migration of sympathetic preganglionic neurons in the spinal cord, where it seems to act as a barrier to neuronal migration. Binding to the extracellular domains of lipoprotein receptors VLDLR and ApoER2 induces tyrosine phosphorylation of Dab1 and modulation of Tau phosphorylation. Reelin can signal through the VLDL receptor and the ApoE2 receptor and can be found circulating in the plasma. 3533 is potentially associated with VLDL and LDL and during this association the protease activity of 3533 serves to degrade the ECM and allow transvessel migration of LDL and VLDL. Once associated within an artery wall, 3533 continues to degrade the ECM and destabilize a developing plaque. Threfore, inhibition of 3533 will reduce the size of atherosclerotic plaques and will increase the stability of already formed plaques. This combination of effects will reduce the number of coronary events. Due to 3533 mRNA expression in the dorsal root ganglion, spinal cord, brain, and liver, along with its functional role, modulators of 3533 activity would be useful in treating disorders associated with cardiovascular disease, including but not limited to atherosclerosis. 3533 polypeptides of the present invention are useful in screening for modulators of 3533 activity.

Gene ID 9462

The human 9462 sequence (SEQ ID NO:47), known also as microsomal epoxide hydrolase, is approximately 1742 nucleotides long including untranslated regions. The coding sequence, located at about nucleic acid 163 to 1530 of SEQ ID NO:47, encodes a 455 amino acid protein (SEQ ID NO:48).

As determined by TaqMan analysis, 9462 mRNA was upregulated by an MTP inhibitor, cholestyramine, niacin, fibrates, and statins. 9462 mRNA was also expressed in adrenal, liver, ovary, and the CNS.

9462 is microsomal epoxide hydrolase. Epoxides are three-membered rings from enzymatic (e.g. P450 monooxygenase system) or chemical oxidative metabolism of endogenous and xenobiotic compounds. Epoxides are usually unstable in aqueous environments and are chemically reactive. Some epoxides are carcinogenic initiators. Epoxide hydrolases convert epoxides to dihydrodiol products. Microsomal epoxide hydrolase catalyzes the hydrolysis of a wide variety of aliphatic and arene oxides to trans-dihydrodiols and is found in the ER and nuclear envelope where it is associated with cytochrome P-450 mixed function oxidase system. Oxysterols, which are regulators of lipid metabolism, are degraded by the P-450 system. Oxysterols serve to reduce the levels of cholesterol synthetic enzymes. Therefore, 9462 is involved in the metabolism of oxysterols and inhibiton of this enzyme will increase oxysterol levels and reduce cholesterol synthesis. This will reduce serum cholesterol levels and have a protective effect on coronary artery disease. Due to 9462 mRNA expression in the adrenal, liver, ovary, and the CNS, along with its functional role, modulators of 9462 activity would be useful in treating disorders associated with cardiovascular disease, including but not limited to atherosclerosis. 9462 polypeptides of the present invention are useful in screening for modulators of 9462 activity.

Gene ID 9123

The human 9123 sequence (SEQ ID NO:49), known also as Adomet decarboxylase, is approximately 1805 nucleotides long including untranslated regions. The coding sequence, located at about nucleic acid 249 to 1253 of SEQ ID NO:49, encodes a 334 amino acid protein (SEQ ID NO:50).

As determined by TaqMan analysis, 9123 mRNA was upregulated in the livers of marmosets treated with lipid-lowering therapies such as niacin, probucol, fenofibrate, gemfibrozil, and cerivastatin. 9123 mRNA was also upregulated in the livers of mice during treatment with ciprofibrate.

9123 is Adomet decarboxylase. The combination of a reduction of Adomet synthetase and an increase in 9123 by lipid-lowering therapies should cause a reduction in the levels of Adomet in the liver. Adomet is a metabolic sensor in the liver and levels of Adomet signal the liver to either move towards a metabolic state or towards a repair state. When in the repair state, such as after liver injury by hepatectomy, the liver reduces the secretion of very low density lipoprotein (VLDL) particles. Therefore, partial inhibition of 9123 would orient the liver towards a repair mode and would therefore result in decrease of VLDL secretion. This would be beneficial for the treatment of hyperlipidemia and atherosclerosis. Due to 9123 mRNA expression in various models of lipid metabolism, along with its functional role, modulators of 9123 activity would be useful in treating disorders associated with cardiovascular disease, including but not limited to atherosclerosis. 9123 polypeptides of the present invention are useful in screening for modulators of 9123 activity.

Gene ID 12788

The human 12788 sequence (SEQ ID NO:51), known also as a phospholipase (PLC-beta-4), is approximately 3707 nucleotides long including untranslated regions. The coding sequence, located at about nucleic acid 231 to 3299 of SEQ ID NO:51, encodes a 1022 amino acid protein (SEQ ID NO:52).

As determined by TaqMan analysis, 12788 mRNA was highly expressed in human arteries, veins, failing hearts, colon tumors, prostate tumors, brain cortex and hypothalamus. Further TaqMan analysis indicated that 12788 mRNA demonstrated robust upregulation across human heart failure samples when compared to non-failing ventricles. In rat models, 12788 mRNA was moderately expressed in aortas. In spontaneously hypertensive-stroke prone rat models, 12788 mRNA was upregulated when compared to Wistar Kyoto normotensive controls in 15 week old animals. In addition, 12788 mRNA was upregulated in aortas from rats treated for 3 days with two anti-hypertensive agents, an AT1-receptor blocker and an L-type calcium channel blocker.

12788 is a phospholipase known as PLC-beta-4. 12788 is a member of a family of PLC's that act through G-protein dependent pathways and is expressed in vascular smooth muscle cells (BR J Pharmacol 1996 June; 118:1003-1011). Phospholipase C catalyzes hydrolysis of phosphatidylinositol 4,5-bisphosphate which generates two second messengers, namely 1,4,5-inositol trisphosphate (IP3) and 1,2 diacylglycerol (DAG). These products are known to modulate intracellular calcium flux and activate protein kinase C. This consequently causes vasoconstriction in vascular smooth muscle cells, leading to an enhancement of vascular tone. Therefore, inhibition of 12788 prevents the formation of 1P3 and DAG and promotes vasorelaxation. In addition, antagoning 12788 in heart disease is likely to benefit similar mechanisms, including a reduction in calcium elevation in the failing myocyte. Due to 12788 mRNA expression in human vasculature, along with its functional role, modulators of 12788 activity would be useful in treating disorders associated with cardiovascular disease, including but not limited to heart failure. 12788 polypeptides of the present invention are useful in screening for modulators of 12788 activity.

Gene ID 17729

The human 17729 sequence (SEQ ID NO:53), known also as ERG4/ERG24, is approximately 1443 nucleotides long including untranslated regions. The coding sequence, located at about nucleic acid 127 to 1383 of SEQ ID NO:53, encodes a 418 amino acid protein (SEQ ID NO:54).

As determined by TaqMan analysis, 17729 mRNA was expressed in the pancreas, brain cortex, CHF heart, prostate tumor and fibrotic liver. 17729 mRNA was upregulated by cholestyramine, fibrates, and cerivastatin in marmoset models. Further TaqMan analysis indicated that 17729 mRNA was downregulated by cholesterol feeding in the ApoE knockout mouse models.

17729 is a member of the ERG4/ERG24 family of proteins. 17729 is an integral membrane protein in the endoplasmic reticulum. 17729 is the product of the human TM7SF2 gene. It has been called sterol reductase 1 (SR-1). Expression of SR-1 in yeast yielded no sterol delta-14-, delta-7- or delta-24-reducatase activities (Biochim. Biophys. Acta 2000, 1529:340-356). A 38 kDa protein from bovine liver with similarity to human SR-1 (92% similarity) had functional delta-14-reductase activity (Eur. J. Biochem. 2002, 269:283-290).

The upregulation of 17729 mRNA by lipid-lowering therapies and its downregulation by cholesterol suggests that 17729 is involved in cholesterol synthesis. Although no delta-14-reductase activity has been detected for human SR-1, we suggest that SR-1 is indeed involved in cholesterol synthesis and that it is a functional sterol reductase of undetermined activity. Due to 17729 mRNA upregulation in various models of heart failure, along with its functional role, modulators of 17729 activity would be useful in treating disorders associated with cardiovascular disease, including but not limited to heart failure. 17729 polypeptides of the present invention are useful in screening for modulators of 17729 activity.

Gene ID 65552

The human 65552 sequence (SEQ ID NO:55), known also as metalloprotease disintegrin 15 (ADAMTS15), is approximately 2853 nucleotides long. The coding sequence, located at about nucleic acid 1 to 2853 of SEQ ID NO:55, encodes a 950 amino acid protein (SEQ ID NO:56).

As determined by TaqMan analysis, 65552 mRNA was expressed in a variety of human tissues, including artery, heart, kidney, skeletal muscle, adipose and breast. Additionally, 65552 mRNA expression was dramatically biased in its vessel expression profile to arteries over veins.

65552 is a metalloprotease disintegrin 15, ADAMTS15 (Cal, S.; Obaya, A. J.; Llamazares, M.; Garabaya, C.; Quesada, V.; Lopez-Otin, C.: Cloning, expression analysis, and structural characterization of seven novel human ADAMTSs, a family of metalloproteinases with disintegrin and thrombospondin-1 domains. Gene 283: 49-62, 2002.) The metalloproteinases (MPs) are a class of molecules which includes the recently discovered ADAMs (a disintegrin and metalloproteinase domain) and ADAMTS (a disintegrin and metalloproteinase thrombospondin) families. Metalloproteinases are key mediators of extracellular matrix degradation. The well described role of MPs in cardiovascular disease, coupled with the expression profile and enrichment in arteries, suggest that inhibition of 65552 would result in a protection against vascular injury and atherosclerosis by reducing matrix breakdown. Due to 65552 mRNA expression along with its functional role, modulators of 65552 activity would be useful in treating disorders associated with cardiovascular disease, including but not limited to atherosclerosis. 65552 polypeptides of the present invention are useful in screening for modulators of 65552 activity.

Gene ID 1261

The human 1261 sequence (SEQ ID NO:57), known also as NR41_HUMAN Orphan nuclear receptor HMR, is approximately 2481 nucleotides long including untranslated regions. The coding sequence, located at about nucleic acid 110 to 1906 of SEQ ID NO:57, encodes a 598 amino acid protein (SEQ ID NO:58).

As determined by TaqMan analysis, 1261 mRNA showed very high expression in large arteries and aorta, with lower but significant expression in diseased specimens. Expression of 1261 mRNA was much lower in hemangioma samples consisting largely of angiogenic endothelial cells. Further TaqMan experiments indicated that 1261 mRNA was upregulated by laminar shear stress and by vascular endothelial growth factor (VEGF) individually, and synergistically upregulated when treated with laminar shear stress followed by 3 hours of VEGF treatment. In addition, a timecourse study of 1261 mRNA expression showed strong upregulation at 1 hour of VEGF treatment in statically cultured or shear-stressed cells treated with VEGF, while upregulation was more persistent in shear-stressed cells.

The cell regulation and tissue expression data indicate that 1261 mRNA expression is correlated with vessel stability. High expression of 1261 is related to resistance to the angiogenic effects of VEGF as measured in the tube formation assay. In the shear stress +VEGF experiments, the well-characterized anti-angiogenic factor Meth-1 was regulated similarly to 1261. Reduced expression of 1261 in diseased and angiogenic vessels compared to healthy stable vessels is consistent with this hypothesis. The transient upregulation of 1261 in response to VEGF, TNF-α, IL-1β, and serum addition, conditions mimicking angiogenic/inflammatory stimuli, indicates that 1261 is involved in immediate responses to counter angiogenic stimulation. Therefore, an antagonist of 1261 is useful for inducing therapeutic angiogenesis. Due to 1261 mRNA expression in a variety of vascular tissues, along with its functional role, modulators of 1261 activity would be useful in treating disorders associated with cardiovascular disease, including but not limited to atherosclerosis. 1261 polypeptides of the present invention are useful in screening for modulators of 1261 activity.

Gene ID 21476

The human 21476 sequence (SEQ ID NO:59), known also as galactoside 2-alpha-L-fucosyltransferase 2 (EC 2.4.1.69) (GDP-L-fucose:beta-D-galactoside 2-alpha-L-fucosyltransferase 2) (Alpha(1,2)FT 2) (Fucosyltransferase 2) (Secretor blood group alpha-2-fucosyltransferase), is approximately 1071 nucleotides long including untranslated regions. The coding sequence, located at about nucleic acid 106 to 918 of SEQ ID NO:59, encodes a 270 amino acid protein (SEQ ID NO:60).

As determined by TaqMan analysis, 21476 mRNA was highly expressed in arteries, HUVEC cells, kidney, ovary, and liver fibrosis tissues. 21476 mRNA was upregulated in confluent endothelial cells from multiple tissue sources compared to proliferating controls. Serum starvation of confluent cells further increased 21476 mRNA expression.

21476, also known as galactoside 2-alpha-L-fucosyltransferase 2, is known to be expressed specifically in cells of endodermal origin. One possible interpretation of 21476 function is that lowering its expression in actively proliferating cells leads to an increase in proliferation. However, several other genes with this pattern of regulation in cells have been shown to be upregulated in angiogenic vessels in vivo, suggesting that the opposite interpretation is also possible. Therefore, antagonists of 21476 may either promote or inhibit angiogenesis. Due to 21476 mRNA expression in a variety of vascular tissues, along with its functional role, modulators of 21476 activity would be useful in treating disorders associated with cardiovascular disease, including but not limited to atherosclerosis. 21476 polypeptides of the present invention are useful in screening for modulators of 21476 activity.

Gene ID 33770

The human 33770 sequence (SEQ ID NO:61), known also as Aldehyde dehydrogenase 8 A1 and ALDH12, is approximately 2156 nucleotides long including untranslated regions. The coding sequence, located at about nucleic acid 39 to 1502 of SEQ ID NO:61, encodes a 487 amino acid protein (SEQ ID NO:62).

As determined by TaqMan analysis, 33770 mRNA expression was at highest levels in the liver, followed by kidney and brain tissues. 33770 mRNA was also downregulated by ciprofibrate (0.76 fold, p=0.02) in the livers of mice.

33770 is known as Aldehyde dehydrogenase 8 A1 and ALDH12. 33770 is 43-47% identical at the nucleic acid level to other aldehyde dehydrogenases (ALDH1, 3, 6, and 9) (J Biol Chem 2000, 275:40106-12). 33770 is known to convert retinal to retinoic acid and is the only aldehyde dehydrogenase known to have some specificity for 9-cis retinal over all-trans retinal. Retinoic acids (specifically 13-cis retinoic acid and 9-cis retinoic acid) are known to increase total cholesterol, ILDL-cholesterol, apoB and triglyceride levels and decrease HDL-cholesterol in humans. This unfavorable lipid profile increases the risk of developing atherosclerosis and coronary artery disease. Our data indicate that 33770 mRNA is highly expressed in liver and is downregulated in ciprofibrate in the liver. This data is consistent with a role for 33770 in generating retinoic acid which is known to yield an unfavorable lipid profile. Therefore blocking the synthesis of retinoic acid through the use of 33770 inhibitors may result in a more favorable lipid profile in humans leading to a reduction in coronary artery disease. Due to 33770 mRNA expression in human lipids, along with its functional role, modulators of 33770 activity would be useful in treating disorders associated with cardiovascular disease, including but not limited to heart failure and coronary artery disease. 33770 polypeptides of the present invention are useful in screening for modulators of 3370 activity.

Gene ID 9380

The human 9380 sequence (SEQ ID NO:63), known also as homogentisate 1,2 dioxygenase (HGD), is approximately 1712 nucleotides long including untranslated regions. The coding sequence, located at about nucleic acid 168 to 1505 of SEQ ID NO:63, encodes a 445 amino acid protein (SEQ ID NO:64).

As determined by TaqMan analysis, 9380 mRNA expression was downregulated in the livers of mice treated with ciprofibrate and downregulated in the ApoE knockout mice on a high cholesterol diet. 9380 mRNA was upregulated in the livers of marmosets treated with microsomal triglyceride transfer protein (MTP) inhibitor, cholestyramine, niacin and probucol.

9380 is homogentisate 1,2 dioxygenase (HGD), a protein involved in tyrosine and phenylalanine degradation that converts homogentisic acid to maleylacetoacetic acid. The endpoint of tyrosine degradation is production of fumarate and acetoacetate. Fumarate then enters the tricarboxylic acid cycle (TCA cycle) for ATP production. Acetoacetate is one of the main ketone bodies synthesized by the liver; ketone bodies are lipid-based energy-yielding molecules that are converted to acetoacetyl CoA in non-liver tissues such as cardiac and skeletal muscle to produce acetyl CoA which in turn enters the TCA cycle to eventually produce ATP. In the liver, acetyl CoA can be converted to HMG CoA to either enter the cholesterol synthesis or the ketone bodies synthesis pathway. Therefore, inhibition of 9380 would decrease the levels of fumarate and acetoacetate in the liver. This inhibition would have an effect on 2 levels. Firstly, less fumarate would enter the TCA cycle reducing ATP production therefore increasing the need for energy. Secondly, a reduction in acetoacetate would result in a decrease in ketone bodies availability for peripheral tissues, a reduction of energy metabolism and therefore an increase in energy demand. Overall, the acetyl CoA that is also required for cholesterol synthesis will be mostly redirected towards the TCA cycle to compensate for a reduction in energy metabolism resulting in a decrease in cholesterol synthesis. Due to 9380 mRNA upregulation in various models of heart failure, along with its functional role, modulators of 9380 activity would be useful in treating disorders associated with cardiovascular disease, including but not limited to hyperlipidemia. 9380 polypeptides of the present invention are useful in screening for modulators of 9380 activity.

Gene ID 2569654

The human 2569654 sequence (SEQ ID NO:65), known also as Betaine-Homocysteine Methyltransferase (BHMT) (E.C. 2.1.1.5), is approximately 2420 nucleotides long including untranslated regions. The coding sequence, located at about nucleic acid 27 to 1247 of SEQ ID NO:65, encodes a 406 amino acid protein (SEQ ID NO:66).

As determined by TaqMan analysis, 2569654 mRNA was significantly downregulated in the livers of ApoE knock out (KO) mice with cholesterol feeding and in mice treated with Ciprofibrate. 2569654 is also downregulated in the livers of LDLR knock out mice fed on a high-fat diet. 2569654 mRNA was also upregulated with T3, Niacin and Cholestyramine treatment in marmoset models.

2569654 or BHMT catalyzes the synthesis of methionine by re-methylating homocysteine using betaine (trimethylglycine) as the methyl donor. In the rat hepatoma cell line, McArdle RH-7777, ApoB mRNA expression is increased significantly with increasing BHMT protein expression (Sowden et al, Biochem. J. (1999) 341: 639-645). In the cell line secreting the highest level of BHMT protein, ApoB lipoprotein secretion was increased. ApoB 48 expression was increased to a greater extent than the increase observed in ApoB 100 expression. Message levels for other apolipoproteins was unaffected by BH expression. In thyroidectomized chickens, overexpression of a 45 KDa protein was observed and later identified as BHMT (Shibata et al, Poult Sci (2003) 82(2):207-213). Expression of BHMT in the liver of the thyroidectomized chickens corresponds to an increase in plasma phospholipid, triglyceride and total cholesterol levels. These data indicate that an increased expression of BHMT increases expression of ApoB mRNA as well as secretion of ApoB lipoprotein. Furthermore, Cholesterol feeding significantly decreased the expression of BHMT in ApoE deficient mice indicating that high levels of cholesterol suppress expression of BHMT in a feedback manner. Ciprofibrate treatment in mice, a therapy used in humans to lower plasma lipids, also downregulates BHMT expression. Our data indictes that the downregulation of BHMT is part of the mechanism of protection by fibrates. Therefore, the inhibition of BHMT leads to the reduction of ApoB, cholesterol and triglyceride levels and could be an effective treatment for hyperlipidemia. Due to 2569654 mRNA expression in highly vascularized tissues, along with its functional role, modulators of 2569654 activity would be useful in treating disorders associated with cardiovascular disease, including but not limited to hyperlipidemia. 2569654 polypeptides of the present invention are useful in screening for modulators of 2569654 activity.

Gene ID 33556

The human 33556 sequence (SEQ ID NO:67), known also as peptide/histidine transporter 2 (PHT2), is approximately 2057 nucleotides long including untranslated regions. The coding sequence, located at about nucleic acid 133 to 1878 of SEQ ID NO:67, encodes a 581 amino acid protein (SEQ ID NO:68).

As determined by TaqMan analysis, 33556 mRNA was expressed in a variety of human tissues, mainly vascularized tissues such as artery, vein, heart, kidney and skeletal muscle. Additionally, 33556 mRNA was highly expressed in macrophages, spleen, and human umbilical vein endothelial cells. 33556 mRNA was also significantly upregulated in atherosclerotic human arteries compared to normal arteries (t-test, p<0.001). In cultured human monocytes and macrophages, 33556 mRNA was robustly expressed and was upregulated at 18h of CD401 and interferon gamma stimulation in macrophages. There was dramatic upregulation of 33556 in ApoE−/−aortic arches when compared to abdominal aortas beginning at 12 weeks of age and continuing through the age of 35 weeks.

33556 is known as peptide/histidine transporter 2 (PHT2) (Herrera-Ruiz, D and Knipp, GT, Journal of Pharmaceutical Sciences, 2003, 92; 691-714). 33556 function has been shown to relate to the transport of dipeptides, with high affinity to histidine. Histidine is converted to histamine by the enzyme histidine decarboxylase. Histadine decarboxlase and its product, histamine, are thought to be causally related to the progression of human atherosclerosis (Higuchi, S, et al, FEBS Letters, 2001, 505: 217-222). Therefore, blocking 33556 would prevent histidine transport into the cytoplasm and would result in a decreased production of histamine. This would result in a decrease in the initiation and degree of atherosclerosis. Due to 33556 mRNA expression in human vasculature, along with its functional role, modulators of 33556 activity would be useful in treating disorders associated with cardiovascular disease, including but not limited to atherosclerosis. 33556 polypeptides of the present invention are useful in screening for modulators of 33556 activity.

Gene ID 53656

The human 53656 sequence (SEQ ID NO:69), known also as an organic anion transporting polypeptide (OATP8) or Liver-specific organic anion transporter 2 LST-2, is approximately 2109 nucleotides long. The coding sequence, located at about nucleic acid 1 to 2109 of SEQ ID NO:69, encodes a 702 amino acid protein (SEQ ID NO:70).

As assessed by TaqMan analysis, 53656 mRNA was downregulated in the livers of African Green Monkeys (1.3 fold). 53656 mRNA was also upregulated by cerivastatin (1.4 fold), cholestyramine, (1.8 fold), niacin (1.4 fold), and the combination therapy of cerivastatin and fenofibrate (2.0 fold) in the livers of Marmosets. In an organ recital model of 40 tissues and cells, 53656 mRNA was found to be highly selective for colon tumor, liver, and liver fibrosis.

Organic Anion Transporting Polypeptides (OATPs) are located on the basolateral (sinusoidal) domain in hepatocytes. They play a role in the removal of organic anions from the circulation (e.g. bile salts [in concert with Na-taurocholate transporters], drugs, some peptide hormones). Each has a unique tissue expression and there is some specificity overlap. Some transporters have unique ligands. Of all the known ligands for OATP8, the only one unique to OATP8 is cholesystkinin-8 (CCK-8). CCK-8 is known to play a role in decreasing gastric emptying (causing appetite suppresion). It also plays a role in increasing gallbladder emptying and exocrine pancreatic secretion. CCK-8 causes a 53% reduction of triacylglycerol secretion in isolated hepatocytes via the CCK-B receptor (Hepatology, 1993, 18:1232-7) and OATP8 selectively removes CCK-8 which leads to its degradation (Hepatology, 1990, 12:301-5).

Inhibition of 53656 or OATP8 leads to increased levels of CCK-8 which decreases triglcyeride secretion by hepatocytes. Increased CCK-8 also increases gall bladder emptying leading to increased bile acid synthesis and reduced liver cholesterol levels. Together this potentially results in a decrease in plasma cholesterol and triglycerides. Due to 53656 mRNA expression in the liver, along with its functional role, modulators of 53656 activity would be useful in treating disorders associated with cardiovascular disease including but not limited to heart failure. 53656 polypeptides of the present invention are useful in screening for modulators of 53656 activity.

Gene ID 44143

The human 44143 sequence (SEQ ID NO:71), known also as Cdc-42 binding kinase beta/MRCK beta, is approximately 6780 nucleotides long including untranslated regions. The coding sequence, located at about nucleic acid 290 to 5425 of SEQ ID NO:71, encodes a 1711 amino acid protein (SEQ ID NO:72).

As determined by TaqMan analysis, 44143 mRNA was expressed predominantly in vascular tissues such as artery, heart, bladder, kidney and in brain cortex. Additionally, 44143 mRNA was expressed in cultured smooth muscle cells and in human umbilical vein endothelial cells. In rats treated with minoxidil or an angiotensin receptor blocker (antihypertensive agents) for twenty eight days, expression of 44143 mRNA was upregulated in aortas compared to vehicle treated controls. In spontaneously hypertensive (SHR) rats, there was upregulation of 44143 mRNA in aortas of the SHR and stroke-prone strains at 15 weeks of age compared to Wistar-Kyoto normotensive age-matched controls.

44143 is known as Cdc-42 binding kinase beta or myotonic dystrophy related kinase beta (MRCKbeta). 44143 is a downstream signaling molecule for the small Rho-like GTPase, Cdc42. Cdc42 is a member of a family of two kinases which effect actin and myosin reorganization (Leung, T et al., Myotonic dystrophy kinase-related Cdc42-binding kinase acts as a Cdc42 effector in promoting cytoskeletal reorganization, 1998, Molecular and Cellular Biology, 18: 130-140). In vitro, the MRCKalpha is shown to phosphorylate myosin light chain, which is known to cause vascular contraction. Therefore, 44143 will have a similar effect as MRCK alpha on the vasculature, hence the inhibition of 44143 will result in vasorelaxation. Due to 44143 mRNA expression in human vasculature, along with its functional role, modulators of 44143 activity would be useful in treating disorders associated with cardiovascular disease including but not limited to vasorelaxation. 44143 polypeptides of the present invention are useful in screening for modulators of 44143 activity.

Gene ID 32612

The human 32612 sequence (SEQ ID NO:73), known also as peptide/histidine transporter 1 (PHT1), is approximately 2699 nucleotides long including untranslated regions. The coding sequence, located at about nucleic acid 181 to 1671 of SEQ ID NO:73, encodes a 496 amino acid protein (SEQ ID NO:74).

As determined by TaqMan analysis, 32612 mRNA was expressed in a variety vascularized tissues such as artery, vein, heart, kidney, skeletal muscle and central nervous system (CNS) structures, such as brain cortex and hypothalamus. Additionally, 32612 mRNA was highly expressed in various cell types, specifically smooth muscle cells, epithelial cells, glial cells and endothelial cells. 32612 mRNA was also upregulated (t-test, p<0.05) in diseased human arteries when compared to normal human arteries.

32612 is known as peptide/histidine transporter 1 (PHT1) (Herrera-Ruiz, D and Knipp, G T, Journal of Pharmaceutical Sciences, 2003, 92; 691-714). The function of 32612 has been shown to relate to the transport of dipeptides and with high affinity to histidine. Histidine is converted to histamine by the enzyme histidine decarboxylase. Histadine decarboxylase and its product, histamine, are thought to be causally related to the progression of human atherosclerosis (Higuchi, S, et al, FEBS Letters, 2001, 505: 217-222). Therefore, blocking the 32612 or PHT1 would prevent histidine transport into the cytoplasm and would result in a decreased production of histamine. This would result in a decrease in the initiation and degree of atherosclerosis. Due to 32612 mRNA expression in human vasculature, along with its functional role, modulators of 32612 activity would be useful in treating disorders associated with cardiovascular disease including but not limited to atherosclerosis. 32612 polypeptides of the present invention are useful in screening for modulators of 32612 activity.

Gene ID 10671

The human 10671 sequence (SEQ ID NO:75), known also as tryptophan 5-monooxygenase (TPH), is approximately 1335 nucleotides long. The coding sequence, located at about nucleic acid 1 to 1335 of SEQ ID NO:75, encodes a 444 amino acid protein (SEQ ID NO:76).

As determined by TaqMan analysis, 10671 mRNA expression was found to be limited to a small number of human tissues. 10671 mRNA was found to be restricted to arteries (normal and diseased) and veins.

10671 is known as tryptophan 5-monooxygenase or TPH. TPH modulates the rate limiting step in the synthesis of serotonin in the central nervous system. This enzyme has been shown to be primarily expressed in CNS structures (pineal gland, dorsal and medial raphe nuclei, gut enterochromaffin cells) (Walther, D J et al., Synthesis of serotonin by a second tryptophan hydroxylase isoform, 2003, Science, 299: 76). Our data is the first to describe the presence of 10671 in blood vessels and the first to support a novel role for the local production of serotonin in peripheral blood vessels. There is a well described role for serotonin in promoting vascular tone and advancing proliferative disorders such as atherosclerosis (Watanabe, T et al., Oxidized low density lipoproteins potentiate the mitogenic effect of 5-hydroxytryptamine on vascular smooth muscle cells, 2002, Jpn Heart J, 43: 35-42. Kandabaski, T et al., Involvement of rho-kinase in agonists-induced contractions of arteriosclerotic human arteries, 2002, 22: 243-248.). Therefore, reducing the levels of serotonin, by inhibiting tryptophan monooxygenase, will promote local vasorelaxation as well as diminish plaque development seen in atherosclerosis. Due to 10671 mRNA expression in human vasculature, along with its functional role, modulators of 10671 activity would be useful in treating disorders associated with cardiovascular disease including but not limited to atherosclerosis. 10671 polypeptides of the present invention are useful in screening for modulators of 10671 activity.

Gene ID 261

The human 261 sequence (SEQ ID NO:77), known also as Kappa-type 3 opioid receptor, is approximately 2534 nucleotides long including untranslated regions. The coding sequence, located at about nucleic acid 118 to 1230 of SEQ ID NO:77, encodes a 370 amino acid protein (SEQ ID NO:78).

As determined by TaqMan analysis, 261 mRNA was expressed in a limited number of human tissues, namely CNS tissues such as artery, vein, heart, kidney and skeletal muscle. 261 mRNA was expressed in mononuclear cells, spleen, and erythroid progenitor cells. 261 mRNA was also significantly upregulated in atherosclerotic human arteries compared to normal tissues (t-test, p<0.05). 261 mRNA was robustly expressed in cultured human monocytes and macrophages. 261 mRNA was also downregulated in monocytes and macrophages treated with interferon gamma and soluble CD40L.

261 is a de-orphaned G-protein coupled receptor known as Kappa-type 3 opioid receptor. Nociceptin has been identified as the ligand of this GPCR. Nociceptin receptors are densely expressed pre and post-synaptically in the nucleus tractus solitarius (Mao L, Wang J Q. Pharmacological activation of nociceptin receptors in the nucleus tractus solitarius inhibits baroreceptor reflex in pentobarbital-anesthetized rats. 2000; Neuroscience, 101:435-40). The activation of these central receptors has been shown to be involved in the regulation of heart rate and blood pressure (Mao L, Wang J Q. Microinjection of nociceptin (Orphanin FQ) into nucleus tractus solitarii elevates blood pressure and heart rate in both anesthetized and conscious rats. 2000; J Pharmacol Exp Ther, 294:255-62). Due to 261 mRNA expression in human vasculature, along with its functional role, modulators of 261 activity would be useful in treating disorders associated with cardiovascular disease including but not limited to atherosclerosis. 261 polypeptides of the present invention are useful in screening for modulators of 261 activity.

Gene ID 44570

The human 44570 sequence (SEQ ID NO:79), known also as ATP-binding cassette, sub-family A (ABC 1), member 6, is approximately 5296 nucleotides long including untranslated regions. The coding sequence, located at about nucleic acid 176 to 5029 of SEQ ID NO:79, encodes a 1617 amino acid protein (SEQ ID NO:80).

As determined by TaqMan analysis, 44570 mRNA was expressed in a variety of human tissues, including artery, vein, heart, liver, brain, ovary, macrophages and breast tissues. 44570 mRNA was strongly expressed in vascular smooth muscle and endothelial cells. 44570 mRNA was also significantly upregulated in failing human myocardium when compared to non-failing hearts (t-test, p=0.005). In Situ hybridization experiments indicated that 44570 mRNA was expressed in human myocytes.

44570 is an ATP-binding cassette, sub-family A (ABC1), member 6, which was cloned as a novel member of the ABC A transporter subfamily from human macrophages.(Kaminski W E, Wenzel J J, Piehler A, Langmann T, Schmitz G. ABCA6, a novel a subclass ABC transporter. Biochem Biophys Res Commun. 2001 Aug. 3;285(5): 1295-301). ATP-binding cassette (ABC) transporters are expressed in monocyte-derived macrophages and are subject to sterol-dependent regulation. ABCA6-like transporters are thought to be involved in macrophage lipid homeostasis. It has been described in the literature that nuclear hormone receptors including LXR/RXR and PPAR/RXR heterodimers act as direct or indirect regulators of other ABC transporters and are potential targets for pharmacological intervention in cardiovascular disease. (Schmitz, G and Drobnik, W, ATP-binding cassette transporters in macrophages: promising drug targets for treatment of cardiovascular disease. Curr Opin Investig Drugs. 2002 June;3(6):853-8). The enriched vascular expression of this target and the regulation in human heart failure suggest a role for ABCA6 in cardiovascular disease. Due to 44570 mRNA expression in highly vascularized tissues, along with its functional role, modulators of 44570 activity would be useful in treating disorders associated with cardiovascular disease, including but not limited to atherosclerosis. 44570 polypeptides of the present invention are useful in screening for modulators of 44570 activity.

Gene ID 41922

The human 41922 sequence (SEQ ID NO:81), known also as blood plasma glutamate carboxypeptidase (PGCP) is approximately 1928 nucleotides long including untranslated regions. The coding sequence, located at about nucleic acid 167 to 1585 of SEQ ID NO:81, encodes a 472 amino acid protein (SEQ ID NO:82).

As determined by TaqMan analysis, 41922 mRNA was expressed in a variety of human tissues and cells including bladder, osteoblasts, veins, CNS structures and pancreas. 41922 mRNA was strongly expressed in normal human arteries and human umbilical vein endothelial cells. 41922 mRNA was significantly upregulated in congestive heart failure samples when compared to non-failing hearts (t-test, p=0.005). In rats treated with antihypertensive agents for 28 days, 41922 was upregulated in aortas compared to the aortas of vehicle treated controls. Furthermore, 41922 was expressed at significantly higher levels in the aortas of spontaneously hypertensive and stroke-prone strains at 15 weeks of age when compared to Wistar Kyoto normotensive age-matched controls. Additionally, there is a trend toward upregulation at a young age (5 wks) suggesting an increase in gene expression which corresponds closely to levels of mean arterial blood pressure. Finally, in rennin transgenic and Sprague Dawley rats, 41922 was upregulated in the aortas of the 15 week old mice when compared to younger 5 week old counterparts, suggesting an age dependent increase.

41922 is a carboxypeptidase also known as PGCP. The specific substrates of 41922 are unknown although it has been shown to function as a glutamate carboxypeptidase, as a dipeptidyl peptidase, as an endopeptidase and an esterase (Gingras R, Richard C, El-Alfy M, Morales C R, Potier M, Pshezhetsky A V. Purification, cDNA cloning, and expression of a new human blood plasma glutamate carboxypeptidase homologous to N-acetyl-aspartyl-alpha-glutamate carboxypeptidase/prostate-specific membrane antigen. J. Biol. Chem. 1999 Apr. 23; 274(17):11742-50). The carboxypeptidase is thought to be responsible for the processing and/or degradation of peptide hormones such as neuropeptides and glutamate. Studies have shown that PGCP is secreted from COS cells and is expressed in blood plasma. The results of the studies described herein indicate that 41922 is highly expressed in arteries and other tissues involved in blood pressure maintenance (veins, HUVECs). Furthermore enhanced expression of 41922 was observed in the hypertensive states. This expression and regulatory data suggest that 41922 is involved in regulating vasomotor tone and heart failure. Therefore modulators of 41922 activities would be useful in treating disorders associated with cardiovascular disease, including but not limited to vasomotor tone and heart failure. 41922 polypeptides of the present invention are useful in screening for modulators of 41922 activity.

Gene ID 2552

The human 2552 sequence (SEQ ID NO:83), known also as glutamyl aminopeptidase (EAP) (EC 3.4.11.7), aminopeptidase A (APA), and differentiation antigen GP160, is approximately 3763 nucleotides long including untranslated regions. The coding sequence, located at about nucleic acid 216 to 3089 of SEQ ID NO:83, encodes a 957 amino acid protein (SEQ ID NO:84).

As determined by TaqMan analysis, 2552 mRNA was expressed in a variety of human tissues and cells including small intestine, colon, hemangiomas, kidney and liver. In marmoset livers 2552 mRNA was upregulated by niacin, cholestyramine, combination therapy with fibrate/statin, and probucol therapy. 2552 mRNA was also upregulated by fibrates in marmoset livers and human hepatocytes. Cirprofibrate was shown to downregulate 2552 mRNA in the livers of mice. In LDLR knock-out mice livers, 2552 mRNA was shown to be downregulated when the mice were fed a high-fat diet.

Thus given that expression of 2552 mRNA is modulated by lipid-lowering therapies as well as high-fat diets, it is apparent that 2552 is modulated in response to lipids and/or bile acid levels in the liver. Furthermore, 2552 is known to cleave CCK8, a peptide hormone that causes gall bladder contraction. (See Migaud et al. The in vivo metabolism of cholecystokinin (CCK-8) is essentially ensured by aminopeptidase A. Peptides. 1996; 17(4):601-607; Shaffer. Review article: control of gall-balder motor function. Ailment Pharmacol. Ther. May 2000;14 Suppl 2:2-8). While not wanting to be bound by theory, it is hypothesized that inhibition of 2552 will lead to increased CCK8 levels which would result in increased gall bladder emptying. Gall bladder emptying removes cholesterol from the body and therefore would result in a decrease in liver cholesterol and a subsequent decrease in plasma cholesterol and triglycerides. Thus given the expression of 2552 is modulated by lipid lowering therapies and high fat diets it is believed that 2552 is involved in cardiovascular disease, including but not limited to atherosclerosis, dyslipidemia and coronary artery disease. 2552 polypeptides of the present invention are useful in screening for modulators of 2552 activity Gene ID 2417

Human 2417, known also as the purinergic receptor P2Y9, is encoded for by the nucleotide sequence of SEQ ID NO:85. The 2417 nucleotide sequence is approximately 1302 nucleotides long including untranslated regions. The coding sequence, located at about nucleic acid residues 152 to 1264 of SEQ ID NO:85, encodes a 370 amino acid protein (SEQ ID NO:86).

As determined by TaqMan analysis, 2417 mRNA was expressed in a variety of human tissues and cells, including platelets, megakaryocytes, HUVECs, spinal cord, brain cortex, brain hypothalamus, breast, ovary and peripheral blood leukocytes. Furthermore, differential expression of 2417 mRNA was found when platelets obtained from normal volunteers were compared to platelets obtained from patients with coronary artery disease (e.g., unstable angina, stable angina, and myocardial infraction). The platelets obtained from patients with coronary artery disease were found to express 2417 mRNA at significantly higher levels than the control platelets.

2417, also known as P2Y9, was classified as a purinergic receptor based upon sequence identity with P2Y receptors. P2Y9 was recently shown to bind lysophosphatidic acid and activate adenylyl cyclase. (See Noguchi et al. (2003) Identification of P2Y9/GPR23 as a Novel G Protein-Coupled Receptor for Lysophosphatidic Acid, Structurally Distant from the Edg Family. J. of Biol. Chem. in pres.). Interestingly, inhibition of adenylyl cyclase is involved in platelet activation and aggregation. (See Kunapuli and Daniel. (1998) Biochem. J.; 336:513-523). For example, inhibitors of platelet aggregation have been shown to block ADP-induced inhibition of adenylate cyclase. (See Kaniel et al. (1998) J. Biol. Chem.; 273:2024-2029).

The invention is based, at least in part, on the discoveries that 2417 is expressed in platelets and megakaryocytes and is expressed at elevated levels in platelets obtained from patients diagnosed with coronary artery disease. The findings herein support the conclusion that 2417 is involved in thrombosis. Furthermore, the recent discovery that P2Y9 activates adenylyl cyclase supports the findings herein that 2417 is involved in thrombosis. Thus, 2417 polypeptides of the present invention are useful in screening for modulators of 2417 and modulators of 2417 would be useful in treating thrombotic disorders.

Gene ID 19319

The human 19319 sequence (SEQ ID NO:87), known as Pantothenate kinase 1 (EC 2.7.1.33) (Pantothenic acid kinase 1) (hPanK1) (hPanK), is approximately 2510 nucleotides long including untranslated regions. The coding sequence, located at about nucleic acid 152 to 1096 of SEQ ID NO:87, encodes a 314 amino acid protein (SEQ ID NO:88).

As determined by TaqMan analysis, 19319 mRNA was expressed in a variety of human tissues, including liver, kidney, skeletal muscle and brain. 19319 mRNA was upregulated in the livers of mice administered ciprofibrate. 19319 mRNA was downregulated by cholesterol in ApoE knock-out mice and downregulated in LDLR knock-out mice administered a high-fat diet. 19319 mRNA is also upregulated by cholestyramine and cerivistatin in the livers of marmosets.

19319 or PANK1 is a Pantothenate kinase. There are four members of the PANK family of genes, namely PANK1, PANK2, PANK3, and PANK4. Pantothenate kinases are known to play a role in regulating intracellular CoA concentration. The PANK enzymes phosphorylate pantothenate, generating D-4′-phosphopantothenate and catalyze the phosphorylation of N-pantothenoyl-cyteine and pantetheine. Pantetheine is hydrolyzed by vanin-1, an ectoenzyme, generating pantothenate and cysteamine. Pantethine is a lipid-lowering drug and it is believed that the breakdown of Pantetheine by vanin-1 generates the active agent, cysteamine, responsible for the lipid-lowering effect. (Atherosclerosis. 1987; 68(1-2):41-49). It is also known that cysteamine is found naturally in the liver. (FEBS Letters 483 (2000) 149-154). The present inventors believe that inhibition of PANK1 will be beneficial to lowering cholesterol. While not wanting to be bound by theory, it is apparent from the teachings present herein that 19319 responds to lipid levels, e.g., 19319 is upregulated by ciprofibrate in mouse livers and downregulated by cholesterol administration in ApoE knockout mice and by high fat diets in LDLR knockout mouse livers. Furthermore, the expression pattern of 19319 (e.g., liver) is consistent with 19319's involvement in dyslipidemia. Additionally 19319 diminishes the level of pantetheine by hydrolysis, reducing the level of cysteamine in the liver. Therefore inhibition of 19319 would lead to an increase in the level of cysteamine which would reduce serum cholesterol and triglycerides. Additionally inhibition of PANK1 should also lead to a decrease in the level of CoA available in the liver for the production of HMGCoA, a cholesterol precursor. Inhibition of PANK1 should also lead to a decrease in the generation of acyl-carrier protein, an important agent in the generation of fatty acids. Thus, 19319 polypeptides of the present invention are useful in screening for modulators of 19319 and modulators of 19319 can be beneficial for lowering cholesterol and triglycerides. Therefore, 19319 modulators can be used to ameliorate cardiovascular disease, including but not limited to atherosclerosis, and coronary artery disease.

Gene ID 43969

The human 43969 sequence (SEQ ID NO:89), known also as HAOX1, Glycolate oxidase or GOX is approximately 1136 nucleotides long including untranslated regions. The coding sequence, located at about nucleic acid 24 to 1136 of SEQ ID NO:89, encodes a 370 amino acid protein (SEQ ID NO:90).

As determined by TaqMan analysis, 43969 mRNA was expressed almost exclusively in the liver. 43969 was differentially expressed in several mouse models used in assessing lipid levels. For example 43969 was downregulated in the livers of mice administered ciprofibrate, ApoE knock-out mice administered cholesterol and in LDLR knock-out mice maintained on a high-fat diet.

43969 or Glycolate oxidase, is an enzyme that oxidizes 2-hydroxyacids with a preference for glycolate, but which can also utilize 2-hyudroxy fatty acids. Additionally 46476 (SEQ ID NO:129, 130) is a glyoxylate reductase and catalyzes the reverse reaction. Studies have shown that glycolate feeding has been shown to increase cholesterol and triglycerides by an unclear mechanism (Pharmacol Res. 1993, 27:289-97) and hyperlipidemia in rats fed a cholesterol- and fat-rich diet was associated with hyperoxaluria (Urol Res. 2000, 28:404-15). Given that glyoxylate can be converted to oxalate, it is therefore likely that the lipid-raising effect of glycolate is via glyoxylate. Based on known information regarding glyoxylate and based on our data demonstrating regulation of genes in the pathway, we hypothesize that glyoxylate and/or glycolate are metabolic sensors in the liver. Our data indicate that ciprofibrate causes gene changes that would lead to a decrease in the levels of glyoxylate and an increase in glycolate. We hypothesize that PPAR alpha agonists may improve lipid profiles, in part, by releasing the inhibitory effect of glyoxylate on beta-oxidation. We hypothesize that it is the conversion of glycolate to glyoxylate that causes an increase in liver cholesterol and triglycerides. Therefore, inhibition of 43969 will lead to a decrease of both triglycerides and cholesterol in the liver by limiting the conversion of glycolate to glyoxylate. Thus, 43969 polypeptides of the present invention are useful in screening for modulators of 43969 and inhibitors of 43969 can be used to ameliorate cardiovascular disease, including but not limited to atherosclerosis, dyslipidemia and coronary artery disease.

Gene ID 8921

The human 8921 sequence (SEQ ID NO:91), known also as ADHP_HUMAN Alcohol dehydrogenase class II pi chain precursor (ADH4) (EC 1.1.1.1) or alcohol dehydrogenase 2 (ADH2), class II, is approximately 1981 nucleotides long including untranslated regions. The coding sequence, located at about nucleic acid 62 to 1240 of SEQ ID NO:91, encodes a 392 amino acid protein (SEQ ID NO:92).

As determined by TaqMan analysis, 8921 mRNA was expressed mainly in the liver with much lower expression in the small intestine and skin. 8921 was downregulated in LDL receptor knockout mice maintained on a high fat diet (1.25% cholesterol +0.5% sodium cholate) and Western diet (0.15% cholesterol) when compared with LDL receptor knockout mice maintained on a normal chow diet (10 mouse liver samples per group). 8921 mRNA was found to be downregulated 0.76−(p=0.02) and 0.77-(p=0.005) fold on the high fat and Western diet, respectively. Using microarray experiments, 8921 mRNA was found to be downregulated by 0.59-fold, p=0.002 when comparing liver samples of mice treated with ciprofibrate with control liver samples. In marmosets treated with cholestyramine and cerivastatin, there was a marked increase in 8921 mRNA expression (1.5- and 1.4- fold, respectively).

Human ADH2 belongs to the alcohol dehydrogenase family composed of five classes and seven isoforms. Unlike other ADHs, ADH2 is expressed mainly in the liver. ADHs are thought to metabolize a wide variety of substrates, including ethanol, retinol, other aliphatic alcohols, hydroxysteroids, and lipid peroxidation products. Although the 7 enzymes are similar in amino acid sequence and in structure, the ADHs differ in their preferred substrates. ADH2 has lower affinity for ethanol than other ADHs but at intoxicating levels the enzyme accounts for 40% of the total ethanol oxidation rate. It exhibits high activity for oxidation of long-chain aliphatic alcohols and aromatic alcohols, converting alcohols to aldehydes for synthesis of fatty acids. Reports also indicate that ADH2 is second only to ADH4 for efficient oxidation of all-trans-retinol. ADH2 is expressed in the liver and preferably converts retinol to retinal over ethanol. However at high levels of ethanol, ADH2 is responsible for 40% of ethanol oxidation. Ethanol is a competitive inhibitor against retinol for ADH2 and it is hypothesized that at higher levels of alcohol in the liver, ADH2 oxidizes ethanol rather than retinol. Further support is evident by the finding that alcohol consumption has been shown to result in elevated HDL-cholesterol levels and reduce the risk of myocardial infraction. Furthermore studies indicate that synthetic retinoids adversely affect plasma cholesterol levels in humans resulting in increased total cholesterol and LDL-cholesterol levels, and decreased HDL-C. In addition, ADH2 activity in humans is significantly higher in males than in females; males are also known to have lower HDL-C than females. It is therefore likely that ADH2 is responsible for the beneficial effects of ethanol consumption on HDL-C profile resulting in a decrease of retinol oxidation. Thus given that expression of 8921 mRNA is modulated by lipid-lowering therapies as well as high-fat diets, and the studies to date, it is apparent that 8921 is modulated in response to lipids and/or bile acid levels in the liver. Therefore, inhibiting ADH2 would have a beneficial effect on lipid profiles by way of a decrease in retinol oxidation and inhibitors of ADH2 could be used to treat cardiovascular disease, including but not limited to atherosclerosis, dyslipidemia and coronary artery disease. Thus, 8921 polypeptides of the present invention are useful in screening for modulators of 8921 and modulators of 8921 would be useful in treating cardiovascular disease, including but not limited to atherosclerosis, dyslipidemia and coronary artery disease.

Gene ID 8993

The human 8993 sequence (SEQ ID NO:93), is an ADP-ribosylarginine hydrolase and is approximately 1365 nucleotides long including untranslated regions. The coding sequence, located at about nucleic acid 106 to 1179 of SEQ ID NO:93, encodes a 357 amino acid protein (SEQ ID NO:94).

As determined by TaqMan analysis, 8993 mRNA was expressed at highest levels in megakaryocytes generated in vitro. Significant 8993 mRNA expression was also observed at high levels in the spleen, peripheral blood monocytes and B cells. 8993 mRNA was detected at significantly higher levels in the platelets of patients with unstable angina or acute coronary syndrome than in platelets from patients with no coronary artery disease.

Mono-ADP-ribosylation of arginine is a reversible modification of proteins with NAD:arginine ADP-ribosyltransferases and ADP-ribosylarginine hydrolases catalyzing the opposing reactions in the cycle. (Journal of Biological Chemistry. 1993. 268(24): 17837). The intracellular ADP-ribosylation enzymes are implicated in cell-signaling (EMBO J. 2003. 22(9):1953). 8993 does not have a predicted signal peptide and therefore is probably not a secreted enzyme. Finally, cytoskeletal components, such as actin, are potential targets for ADP-ribosylation. ADP ribosylation of actin would inhibit cytoskeletal reorganization, an early event in platelet activation.

The precise cellular target of ADP-ribosylarginine hydrolase is not known, however inhibition of ADP-ribosyl hydrolase would result in increased adenylyl cyclase activation and subsequent increased cyclic AMP levels, which results in the inhibition of platelet degranulation. Either independent of the increased cyclic AMP, or in parallel, ADP-ribosyl hydrolase inhibition may also block the GPCR signaling and cytoskeletal reorganization that is necessary for platelet degranulation. The significant increase in levels of 8993 mRNA in the platelets of patients with unstable angina, as compared with patients without coronary artery disease, suggests a possible role for 8993 in thrombus formation. Thus, based upon the teachings present herein particularly the elevated expression of 8993 mRNA observed in patients with coronary artery disease when compared with those without coronary artery disease, it is the present invention that 8993 is involved in thrombus formation and inhibition of 8993 would inhibit platelet aggregation and thrombus formation. 8993 polypeptides of the present invention are useful in screening for modulators of 8993 activity.

Gene ID 955

The human 955 sequence (SEQ ID NO:95), is known as PTP-PEST, a human non-transmembrane protein tyrosine phosphatase, and is approximately 3425 nucleotides long including untranslated regions. The coding sequence, located at about nucleic acid >1 to 2619 of SEQ ID NO:95, encodes a 872 amino acid protein (SEQ ID NO:96).

As determined by TaqMan analysis, 955 mRNA was highly expressed in brain and megakaryocytes. Additionally 955 mRNA was highly expressed in human platelets.

955 is a protein tyrosine phosphatase that is known to be associated with focal adhesion proteins and has been implicated in cell migration via the action of integrins. (J. Cell Sci. 2002; 115(Pt22):4305). Furthermore 955 has been shown to interact with the PDGF receptor. (Biochemistry. 2003; 42(9):2691). The studies presented herein teach that 955 is expressed in megakaryocytes and human platelets. While not wanting to be bound by theory, inhibition of PTP-PEST would provide a means to inhibit platelet activation and thrombus formation. Thus inhibitors of 955 can be used to treat arterial thrombosis. 955 polypeptides of the present invention are useful in screening for modulators of 955 activity.

Gene ID 32345

The human 32345 sequence (SEQ ID NO:97), known as duet or trad kinase (Gene. 199; 227:249-255) is approximately 5355 nucleotides long including untranslated regions. The coding sequence, located at about nucleic acid 118 to 3987 of SEQ ID NO:97, encodes a 1289 amino acid protein (SEQ ID NO:98).

As determined by TaqMan analysis, 32345 mRNA was most highly expressed in arteries (normal and diseased), HUVECs, skeletal muscle, CNS structures and megakaryocytes. In addition, 32345 mRNA was upregulated by mevastatin, an HMG-CoA reductase inhibitor, in HUVECs. In rat aortas, 32345 mRNA was upregulated in SHRs and SHR-sps with age compared to WKY controls. Upregulation of 32345 mRNA was also observed in the presence of 3 day or 28 day treatment with anti-hypertensives.

The cellular localization of 32345 shows overlap with actin-cytoskeletal elements. In general, Dbl-homology guanine nucleotide exchange factors (DH-GEFs) regulate actin cytoskeletal reorganization, cell adhesion, and gene transcription via activation of Rho GTPases. Trio, which is closely related to 32345, is a unique Rho GEF, because it has separate GEF domains, GEFD1 and GEFD2 that control the GTPases RhoG/Racl and RhoA, respectively. It is the present invention that inhibitors of 32345 will result in decreased vascular tone and blood pressure. The invention is based at least in part on the studies indicating that 32345 is homologous to Trio, the studies first disclosed herein indicating 32345 mRNA is expressed in vascular tissues, is regulated in HUVECs by a Rho inhibitor and in rat aortas from tone models. Thus inhibitors of 32345 can be used to treat cardiovascular disease, specifically those indications in which vascular tone and blood pressure is a problem. 32345 polypeptides of the present invention are useful in screening for modulators of 32345 activity.

Gene ID 966

The human 966 sequence (SEQ ID NO:99), known as R-PTP-epsilon is approximately 2160 nucleotides long including untranslated regions. The coding sequence, located at about nucleic acid 52 to 2154 of SEQ ID NO:99, encodes a 700 amino acid protein (SEQ ID NO:100). 966 is a protein tyrosine phosphatase epsilon that was identified as having both a cytoplasmic and transmembrane form. The cytoplasmic form is induced by LPS and is thought to be expressed in macrophages.

As determined by TaqMan analysis, 966 mRNA was upregulated in human diseased arteries compared to normal vessels. Additionally, there was significant upregulation in ApoE knockout mouse aortas at various ages compared to wild-type counterparts. The upregulation of 966 mRNA in mice aortic arches was inhibited when captopril, an ACE inhibitor and anti-atherogenic drug, was given to the mice for 6 or 12 weeks.

The invention is based at least in part on studies which demonstrate R-PTP-epsilon dephosphorylates interleukins in macrophages, which inhibits IL-6 and IL-10 induced JAK/STAT signaling. IL-10 is a known anti-atherogenic factor and when overexpressed can reduce the progression of atherosclerosis in mouse models and conversely, IL-10 deficiency promotes atherogenesis. Additionally the studies disclosed herein demonstrate differential expression in diseased arteries and ApoE knockout mice aortas. Therefore, inhibition of 966 will specifically prevent the inactivation of IL-10 and promote JAK/STAT signaling. It is the present invention that inhibition of 966 would inhibit macrophage activation and plaque development during the formation of atherosclerotic lesions and therefore inhibitors of 966 can be used to treat cardiovascular disorders including but not limited to atherosclerosis. 966 polypeptides of the present invention are useful in screening for modulators of 966 activity.

Gene ID 1920

The human 1920 sequence (SEQ ID NO:101), known as glutamate receptor 2 or AMPA2, is approximately 3331 nucleotides long including untranslated regions. The coding sequence, located at about nucleic acid 161 to 2812 of SEQ ID NO:101, encodes an 883 amino acid protein (SEQ ID NO:102).

As determined by TaqMan analysis using a human tissue panel, 1920 mRNA expression was fairly restricted in human tissues, with the most abundant expression observed in brain cortex, followed by hypothalamus, pituitary gland, diseased aorta, dorsal root ganglion and normal artery. In SHR and SHR-sp aortas, 1920 mRNA was dramatically down-regulated (p<0.001) prior to and during the hypertensive state (at 5 and 15 weeks of age) compared to WKY controls. 1920 mRNA was also significantly expressed in rat mesenteric vascular bed.

L-glutamate acts as an excitatory neurotransmitter at many synapses in the central nervous system. The postsynaptic actions of GLU are mediated by a variety of receptors that are named according to their selective agonists. This receptor binds AMPA(quisqualate)>glutamate>kainate. There are two splice isoforms, called “flip” and “flop” which are created by two alternative, adjacent exons preceding the 4th transmembrane domain. The two isoforms have different function, pharmacology and localization. In addition, 1920 is subject to post-translational RNA editing of one nucleotide (and hence one amino acid) which can also change functional properties.

Regulation of vascular tone by glutamate is a novel disease hypothesis. The presence of glutamate receptors (1920) in the vasculature and the regulation of 1920 and correlation of expression of 1920 with a hypertensive state suggests a role for glutamate signaling in the cardiovascular system which is independent of its role in neural transmission. Due to 1920 mRNA expression, along with its functional role, modulators of 1920 activity would be useful in treating disorders associated with cardiovascular disease, including but not limited to atherosclerosis. 1920 polypeptides of the present invention are useful in screening for modulators of 1920 activity.

Gene ID 17318

The human 17318 sequence (SEQ ID NO:103), known as GPR41, is approximately 1830 nucleotides long including untranslated regions. The coding sequence, located at about nucleic acids 449 to 1786 of SEQ ID NO:103, encodes a 445 amino acid protein (SEQ ID NO:104).

As determined by TaqMan analysis, 17318 mRNA was expressed in a variety of human tissues, including blood vessels, kidney, heart, tumors of the colon, lung, breast and prostate and also in CNS structures. Additionally, it is expressed in macrophages, megakaryocytes, and erythroid progenitor cells. It is also well expressed and significantly upregulated in atherosclerotic human arteries (p<0.05; t-test).

17318 or GPR41 was recently de-orphaned as a receptor for propionate and butyrate (short chain fatty acids, SCFAs) (Brown et al., JBC, 2003; 278: 11312-11319). SCFAs have been shown to have an anti-inflammatory effect in vitro and in vivo (Cavaglieri et al., Life Sci. 2003 Aug. 15;73(13):1683-90 and Rodriguez-Cabezas et al., J Nutr. 2002 November;132(11):3263-71). Some of the mechanisms associated with this are reductions in IL-2 production in lymphocytes, decreased TNFαand nitric oxide production and increased IL-10 production. Based in part on the fact that the presence of pro-inflammatory mediators such as NO and interleukins are a determinant of the disease process in vessel wall atherosclerosis, we hypothesize that agonism of this SCFA-stimulated GPCR would result in a reduction of the macrophage inflammatory response and cytokine release in atherosclerotic plaques and thus diminished lesion size. Thus, inhibitors of 17318 can be used to ameliorate cardiovascular disease, including but not limited to atherosclerosis and coronary artery disease. 17318 polypeptides of the present invention are useful in screening for modulators of 17318 activity.

Gene ID 1510

The human 1510 sequence (SEQ ID NO:105), known as mitogen-activated protein kinase kinase kinase 8, or serine/threonine-specific protein kinase, cot [cancer osaka thyroid oncogene], 58K form; or, tumor progression locus 2 (tpl2), is approximately 2763 nucleotides long including untranslated regions. The coding sequence, located at about nucleic acids 367 to 1770 of SEQ ID NO:105, encodes a 467 amino acid protein (SEQ ID NO:106).

As determined by TaqMan analysis, 1510 mRNA was upregulated in diseased human vessels when compared to normal vessels. Furthermore, 1510 mRNA was upregulated in the aortic arches of ApoE knockout mice relative to abdominal aorta, at 12 weeks. This elevated expression can be blocked with the angiotensin converting enzyme inhibitor, captopril.

1510, known as mitogen-activated protein kinase kinase kinase 8, or serine/threonine-specific protein kinase, cot [cancer osaka thyroid oncogene], 58K form; or, tumor progression locus 2 (tpl2), contains a protein kinase domain and has been shown to have serine/threonine kinase activity (Aoki M, Hamada F, Sugimoto T, Sumida S, Akiyama T, Toyoshima K.; J. Biol. Chem. 1993 Oct. 25;268(30):22723-32). Inhibitors of 1510 would be expected to block NFκB mediated inflammatory responses in macrophages. There may also be a role for this kinase in activation of ERK and other MAPK (e.g. JNK and p38), which are also known to participate in the inflammatory process in macrophages (e.g. production of the pro-inflammatory cytokine, TNFα). Repression or dampening of the inflammatory response in macrophages would be expected to hinder or halt the progression of atherosclerosis in humans. Thus, inhibitors of 1510 can be used to ameliorate cardiovascular disease, including but not limited to atherosclerosis and coronary artery disease. 1510 polypeptides of the present invention are useful in screening for modulators of 1510 activity.

Gene ID 14180

The human 14180 sequence (SEQ ID NO:107), known as Human Rac-gamma serine/threonine protein kinase (EC 2.7.1.-) (RAC-PK-gamma) (Protein kinase Akt-3) (Protein kinase B, gamma) (PKB gamma), is approximately 2811 nucleotides long including untranslated regions. The coding sequence, located at about nucleic acids 1 to 1440 of SEQ ID NO:107, encodes a 479 amino acid protein (SEQ ID NO:108).

As determined by TaqMan analysis, 14180 mRNA was upregulated in diseased human vessels compared to normal vessels. Significant 14180 mRNA levels were expressed in the brain.

Human 14180 or Human Rac-gamma serine/threonine protein kinase (EC 2.7.1.-) (RAC-PK-gamma) (Protein kinase Akt-3) (Protein kinase B, gamma) (PKB gamma), is one of three members of the AKT/PKB family of serine/threonine kinases (AKT1, PKB alpha; AKT2, PKB beta; AKT3, PKB gamma). Each is expressed with distinct tissue distribution and shares over 80% amino acid similarity. PKB/Akt is activated in cells exposed to diverse stimuli such as hormones, growth factors, and extracellular matrix components. PKB/Akt phosphorylates and regulates the function of many cellular proteins involved in processes that include metabolism, apoptosis, and proliferation. The increased activity in leukocytes seems to be largely progrowth (it inactivates pro-apoptotic genes). C(2)-ceramide was suggested to inhibit LPS-induced NO production through down-regulating the activation of Akt in RAW 264.7. IFN-gamma induces the phosphorylation of the serine/threonine kinase Akt in primary human peripheral blood monocytes, and abrogation of the IFN-stimulated Akt activation by phosphatidylinositol-3 kinase (PI-3K) inhibitors prevents IFN-induced adhesion in these cells. An inhibitor of this kinase would therefore be expected to block inflammatory processes in macrophages. Such repression, or dampening of the inflammatory response in macrophages would be expected to hinder, or halt the progression of atherosclerosis in humans. Therefore inhibitors of 14180 would be useful to ameliorate cardiovascular disease, including but not limited to atherosclerosis and coronary artery disease. 14180 polypeptides of the present invention are useful in screening for modulators of 14180 activity.

Gene ID 26005

The human 26005 sequence (SEQ ID NO:109), is an adenosine deaminase and is approximately 3941 nucleotides long including untranslated regions. The coding sequence, located at about nucleic acids 213 to 1748 of SEQ ID NO:109, encodes a 511 amino acid protein (SEQ ID NO:110).

As determined by TaqMan analysis, 26005 mRNA was upregulated in diseased human vessels when compared to normal vessels.

Human 26005 shows sequence similarity to insect and mollusk growth factors. The N terminus contains a secretory signal peptide and the C-terminal half shows significant similarity to adenosine deaminase (ADA), particularly in the conserved catalytic domain. Adenosine deaminase inhibitors are found to have utility in regulating inflammation, hypertension, and ischemic injury. An inhibitor of this enzyme would thereore be expected to elevate the levels of adenosine and thus be useful to ameliorate cardiovascular disease, including but not limited to atherosclerosis and coronary artery disease. 26005 polypeptides of the present invention are useful in screening for modulators of 26005 activity.

Gene ID 554, 16408 and 42028

The cholesterol synthesis pathway has been fairly well described and the proteins, or their enzymatic activities, in this pathway are almost all known. In contrast, the pathways that modulate VLDL synthesis (ApoB synthesis, phosphatidylcholine synthesis, triglyceride synthesis) are less well known. Through the use of lipid-altering paradigms, we have found a pathway whose genes are highly regulated by cholesterol/lipid altering treatments. This overall pathway has not been described as being a crucial component of VLDL synthesis and therefore has not been described as being a target for inhibition for dyslipidemias. Each of the individual genes in isolation or combination may be a therapeutic target for a small molecule inhibitor.

The pathway specifically in the liver involves first the flux of betaine in the liver either by conversion of betaine aldehyde to betaine (via betaine aldehyde dehydrogenase) or by the uptake of betaine into the liver via 554 (Sodium- and chloride-dependent betaine transporter; also called gamma-aminobutyric acid transporter (BGT-1)). Betaine can be ingested in the diet. The foods with the most abundant betaine concentration (mg/100 g) are: wheat bran (1339), wheat germ (1241), spinach (645), pretzels (237), shrimp (218) and wheat bread (201). Betaine is then combined with homocysteine by betaine-homocysteine methyltransferase (BHMT) and is converted to methionine and dimethylglycine. The methionine is converted to S-adenosylmethionine (SAM) via MAT1a and SAM is a methyl donor to generate phosphatidylcholine (PC) from phosphatidylethanolamine (PE) via phosphatidylethanolamine methyltransferase (PEMT). Part of this pathway has been described for PC biosynthesis for bile production (Breslow et al, 2003, J. Lipid Res, June 16), but has not been clearly shown to play a role in regulation of plasma cholesterol levels. The dimethylglycine that is formed from BHMT is converted to sarcosine and then to glycine. The conversion of sarcosine to glycine is mediated by 42028 and the reverse reaction is mediated by 16408 disclosed herein. Glycine is then cleaved in the mitochondria to CO₂ via the glycine cleavage system. Cysteamine, a potent inhibitor of the glycine cleavage system, can block glycine cleavage. Therefore, increased flux of methyl groups through the betaine pathway yields a higher level of PC production and thus a higher level of plasma VLDL and cholesterol (refer to Am J Clin Nutr, 2002, 76:961-967; J Nutr Sci Vitaminol (Tokyo). 1986, 32:537-49; J Nutr Sci Vitaminol (Tokyo). 1990, 36 Suppl 2:S105-10; J Nutr. 1999, 129:1807-13; J Nutr Sci Vitaminol (Tokyo). 1986, 32:537-49; J Nutr Sci Vitaminol (Tokyo). 1990, 36 Suppl 2:S105-10; Int J Clin Pharmacol Ther Toxicol. 1986;24:630-7; Clin Ter. 1989;128:411-22; Acta Biomed Ateneo Parm 1987;58: 143-52).

Gene ID 554

The human 554 sequence (SEQ ID NO:111), a sodium- and chloride-dependent betaine transporter (Na⁺/Cl— betaine/GABA transporter); also called gamma-aminobutyric acid transporter (BGT-1), is approximately 1920 nucleotides long including untranslated regions. The coding sequence, located at about nucleic acids 61 to 1905 of SEQ ID NO:111, encodes a 614 amino acid protein (SEQ ID NO:112).

As determined by TaqMan analysis using a human tissue panel, 554 mRNA is highly expressed in kidney, brain cortex, and liver.

As determined by microarray profiling experiments comparing LDL receptor knockout mice on a high fat diet (1.25% cholesterol +0.5% sodium cholate) and on a Western diet (0.15% cholesterol) with LDL receptor knockout mice on a normal chow diet (10 mouse liver samples per group), 554 mRNA was downregulated in the LDL receptor knockout mice on a high fat diet and on a Western diet. 554 mRNA was found to be down 0.65-fold due to high fat and Western diet (p<0.001). Additional microarray experiments performed on the livers of mice treated with ciprofibrate demonstrated that 554 mRNA was downregulated by 0.68-fold (p=0.006) in the livers of the mice treated with ciprofibrate compared to control liver samples. 554 mRNA was also shown to be down-regulated by cholesterol in the livers of African Green Monkeys.

Additional microarray profiling experiments were conducted which compared the expression patterns of 554 mRNA in ApoE knockout mice maintained on a Western diet for 0, 2 and 6 months. Results demonstrated that 554 mRNA was downregulated by 0.51-fold (p<0.001) at 6 months on the Western diet compared to the 0 month time point.

554 belongs to the sodium:neurotransmitter symporter (SNF) family that has a common structure of 12 presumed transmembrane helices and includes carriers for GABA, noradrenaline/adrenaline, dopamine, serotonin, proline, glycine, choline, betaine and taurine. In a hyperosmotic environment, BGT-1 is upregulated and betaine is transported into cells along with up to 2 Cl⁻ and 3 Na⁺ ions. Studies of betaine supplementation show an increase of betaine in the plasma along with its accumulation in the liver. In humans, studies show that betaine supplemented diets showed a decrease in plasma homocysteine concentration and a small but significant increase of total and LDL cholesterol concentration compared to the control group. Plasma homocysteine is associated with a higher risk of cardiovascular disease. BHMT converts homocysteine and betaine to methionine and dimethylglycine. Methionine is thought to increase lipoprotein synthesis. Therefore, specifically inhibiting 554 would have a dual beneficial effect on atherosclerosis. First, a reduction of betaine transport into the liver would result in a reduction of betaine and methionine conversion by BHMT therefore a reduction of methionine converted to S-adenosylmethionine (SAM) via MATla. SAM is a methyl donor to generate phosphatidylcholine (PC) from phosphatidylethanolamine (PE) via PEMT to generate lipoprotein particles. Thus, a reduction in synthesis and secretion of lipoproteins would result from the inhibition of 554. Secondly, an inhibition of betaine transport into the liver would result in an increase of betaine in the plasma. Since high betaine levels are known to reduce the levels of plasma homocysteine, BHMT also present in erythroid cells may be responsible for converting plasma betaine and homocysteine to methionine and dimethylglycine, therefore having a positive effect by reducing homocysteine levels in the blood.

Therefore, based on the function of 554 and its specific expression and regulation patterns, modulators of 554 activity would be useful in treating cardiovascular disorders including but not limited to atherosclerosis. 554 polypeptides of the present invention would be useful in screening for modulators of 554 activity.

Gene ID 16408

The human 16408 sequence (SEQ ID NO:113), known as glycine N-methyl transferase, is approximately 1097 nucleotides long including untranslated regions. The coding sequence, located at about nucleic acids 11 to 898 of SEQ ID NO:113, encodes a 295 amino acid protein (SEQ ID NO:114).

As determined by TaqMan analysis using a human tissue panel, 16408 mRNA expression was elevated in pancreas, liver, prostate tumor and pituitary samples. Furthermore, microarray profiling experiments demonstrated that 16408 mRNA was down-regulated in the livers of LDLR knock-out mice which were fed a high-fat diet. Additionally, microarray profiling experiments demonstrated that 16408 mRNA down-regulated in the livers of mice treated with ciprofibrate.

16408 is known as glycine N-methyl transferase (GNMT). Glycine N-methyltransferase (GNMT; EC 2.1.1.20) catalyzes the synthesis of N-methylglycine (sarcosine) from glycine using S-adenosylmethionine (AdoMet) as the methyl donor. GNMT acts as an enzyme to regulate the ratio of S-adenosylmethionine to S-adenosylhomocysteine (AdoHcy) and participates in the detoxification pathway in liver cells. Therefore, based on the function of 16408 and its specific expression and regulation patterns, modulators of 16408 activity would be useful in treating cardiovascular disorders including but not limited to atherosclerosis. 16408 polypeptides of the present invention would be useful in screening for modulators of 16408 activity.

Gene ID 42028

The human 42028 sequence (SEQ ID NO:115), known as sarcosine dehydrogenase, is approximately 3363 nucleotides long including untranslated regions. The coding sequence, located at about nucleic acids 259 to 3015 of SEQ ID NO:115, encodes a 918 amino acid protein (SEQ ID NO:116).

As determined by TaqMan analysis using a human tissue panel, 42028 mRNA expression was elevated is pancreas and liver samples, followed by bladder samples. Furthermore, microarray profiling experiments demonstrated that 42028 was down-regulated in the livers of LDLR knock-out mice fed a high-fat diet. Additionally, microarray profiling experiments demonstrated that 42028 was down-regulated in the livers of mice treated with ciprofibrate.

42028 is known as sarcosine dehydrogenase. Sarcosine dehydrogenase (SARDH; EC 1.5.99.1) is a liver mitochondrial matrix flavoenzyme that catalyzes the oxidative demethylation of sarcosine disease. Inhibition of 42028 will cause a decrease in the betaine-homocysteine-methionine conversion pathway and therefore lead to a decrease in VLDL synthesis. Therefore, based on the function of 42028 and its specific expression and regulation patterns, modulators of 42028 activity would be useful in treating cardiovascular disorders including but not limited to atherosclerosis. 42028 polypeptides of the present invention would be useful in screening for modulators of 42028 activity.

We have identified three genes, namely 554, 16408 and 42028 that are regulated in the liver by manipulation of cholesterol levels in animals. These genes highlight a potential pathway that is involved in VLDL synthesis, probably through regulation of phosphatidylcholine synthesis. Therefore, specifically inhibiting a single gene (or combination of these genes) of this pathway in the liver will lead to alterations in plasma cholesterol and hence may be useful in treating cardiovascular disorders including but not limited to atherosclerosis.

Gene ID 112091

The human 112091 sequence (SEQ ID NO:117), known as the Na+ and H+coupled amino acid transport system N, is approximately 2431 nucleotides long including untranslated regions. The coding sequence, located at about nucleic acids 125 to 1639 of SEQ ID NO:117, encodes a 504 amino acid protein (SEQ ID NO:118).

As determined by TaqMan analysis using a human tissue panel, 112091 mRNA expression was elevated in liver samples, followed by pancreas, cortex, spinal cord, hypothalamus and skeletal muscle samples. Furthermore, 112091 is down-regulated by cholesterol in the livers of African Green Monkeys, it is down-regulated in the livers of LDLR knock-out mice fed a high-fat diet and it is down-regulated in the livers of wild-type mice treated with ciprofibrate.

112091 is an amino acid transporter. It is known as solute carrier family 38, member 3, as G17, as SN1, and also as system N1 Na+ and H+-coupled glutamine transporter. It can mediate glutamine efflux as well as uptake. Intake of certain amino acids has an impact on plasma cholesterol levels. For example, methionine raises plasma cholesterol (J Nutr Sci Vitaminol (Tokyo). 1990, 36 Suppl 2:S105-10; J Nutr. 1999, 129:1807-13) and glycine reduces plasma cholesterol (J Nutr Sci Vitaminol (Tokyo). 1986, 32:537-49; J Nutr Sci Vitaminol (Tokyo). 1990, 36 Suppl 2:S105-10). Regulation of 112091 in cholesterol-related models suggests that it may be involved in lipoprotein and cholesterol metabolism. Inhibition of 112091 should result in a decrease in plasma cholesterol levels. 112091 polypeptides of the present invention would be useful in screening for modulators of 112091 activity.

Gene ID 13886

The human 13886 sequence (SEQ ID NO:119), known as MAP/microtubule affinity-regulating kinase 1 (MARK1), is approximately 2831 nucleotides long including untranslated regions. The coding sequence, located at about nucleic acids 160 to 2547 of SEQ ID NO:119, encodes a 795 amino acid protein (SEQ ID NO:120).

As assessed by TaqMan analysis, 13886 mRNA is expressed in a variety of human tissues, with predominance in vascular tissues such as artery, vein, heart, and in brain cortex and pituitary gland. Additionally, it is highly expressed in human umbilical vein endothelial cells. In rats treated with minoxidil, nifedipine or an angiotensin receptor blocker (antihypertensive agents) for three days, expression of 13886 mRNA is dramatically down-regulated in aortas compared to vehicle treated controls (p<0.001; ANOVA). In rats treated with guanylate cyclase stimulators or activators or L-NAME (a nitric oxide synthase inhibitor) for seven days, expression of 13886 mRNA is also significantly down-regulated in aortas compared to vehicle treated controls (p<0.005; ANOVA). In spontaneously hypertensive rats, there is a trend toward upregulation of the kinase in 15 week old aortas compared to WKY normotensive age-matched controls.

13886 is known as MAP/microtubule affinity-regulating kinase 1 (MARK1). It is a member of a family of four MARKs, which phosphorylate microtubule associated proteins, tau and MAPs.(J. Biol. Chem. 1996 May 3;271(18):10834-43.) Microtubules can affect the contractile process in several cell types such as fibroblasts, cardiac myocytes and vascular smooth muscle cells of large and intermediate-sized arteries. One possible mechanism of tone is thought to be the imposition of a viscous intracellular load which is correlated with density of microtubules. This opposes the actin cytoskeleton and contraction. Colchicine, nocodazole, demecolcine all caused significant vasoconstriction in isolated rat arterioles (33.9, 29.6, 32.7% compared to basal tone). (Platts et al., Alteration of microtubule polymerization modulates arteriolar vasomotor tone. Am J. Physiol. 1999 July;277 (1 Pt 2):H100-6). Disruption of microtubules via nocodazole has been shown to cause increases in vascular tension and intracellular calcium levels, which is known to cause vascular contraction in coronary arteries (Am J Physiol Heart Circ Physiol. 2000 November;279(5):H2493-501).

It is believed is that MARK1 via phosphorylation of MAPs will have a similar disruptive effect on microtubules within the vasculature and that inhibiting this kinase will result in vasorelaxation via microtubule stabilization. Thus, inhibitors of 13886 can be used to ameliorate cardiovascular disease, including but not limited to atherosclerosis and coronary artery disease. 13886 polypeptides of the present invention would be useful in screening for modulators of 13886 activity.

Gene ID 13942

The human 13942 sequence (SEQ ID NO:121), known as matrix metalloproteinase 19 (MMP19), is approximately 1811 nucleotides long including untranslated regions. The coding sequence, located at about nucleic acids 102 to 1628 of SEQ ID NO:121, encodes a 508 amino acid protein (SEQ ID NO:122).

As determined by TaqMan analysis, 13942 mRNA is expressed largely in macrophages with some tissue expression found in pancreas, adipose, ovary and kidney. It is also found differentially expressed in human diseased vessels, vs. normal vessels. Additionally, the mouse ortholog of 13942 is found more highly expressed in regions of the aorta (the arch) which contain a higher portion of atherosclerotic lesion, than the abdominal aorta.

The inhibition of this gene would be expected to decrease the amount of insulin-like growth factor (IGF) I and II that are present in the vessels, by preventing the degradation of insulin-like growth factor binding protein (IGFBP) III (and possibly I, II and IV). IGFBP is one of the principal substrates of MMP19. Increased IGFI is a known smooth muscle cell mitogen and chemotactic agent, increasing neoangiogenesis of endothelial cells and increasing recruitment, differentiation and expression of inflammatory cytokines from macrophages. Inhibiting these biological processes in diseased vessels, would be expected to largely attenuate the inflammatory biology, medial thickening (SMC proliferation and migration) and vascularization of the lesion.

Thus, inhibitors of 13942 can be used to ameliorate cardiovascular disease, including but not limited to atherosclerosis and coronary artery disease. 13942 polypeptides of the present invention would be useful in screening for modulators of 13942 activity.

Gene ID 1673

The human 1673 sequence (SEQ ID NO:123), known as KSYK_HUMAN Tyrosine-protein kinase SYK (EC 2.7.1.112) (Spleen tyrosine kinase), is approximately 2639 nucleotides long including untranslated regions. The coding sequence, located at about nucleic acids 148 to 2055 of SEQ ID NO:123, encodes a 635 amino acid protein (SEQ ID NO:124).

As assessed by TaqMan analysis, 1673 mRNA is expressed largely in megakaryocytes, progenitors, erythroid cells and neutrophils, with smaller amounts in macrophages. It is also found in tonsil, pancreas, kidney, lymph node and spleen. 1673 mRNA was found to be differentially expressed in human diseased vessels, vs. normal vessels. 1673 mRNA expression was upregulated in diseased monkey aorta and it was dramatically upregulated in ApoE knock out arch/ab mice. Syk is upregulated by ModLDL in macrophages, but not monocytes. No apparent upregulation was observed by CD40L or IFNg in monocytes or macrophages. Additionally, the mouse ortholog of 1673 is more highly expressed in regions of the aorta (the arch) which contain a higher portion of atherosclerotic lesion than the abdominal aorta.

Inhibitors of 1673 will have profound effects on phagocytosis and leukocyte attachment/recruitment. In addition Syk plays a role in full activation of both NFkB and ERK pathways in macrophages. Thus, an inhibitor of Syk is expected to decrease the load of highly activated macrophages at the site of vascular inflammation and reduce plaque burden. In addition this will likely stabilize the lesion and reduce neovascularization. This would be expected to occur through decreases in the activation of the p65 subunit of NFkB, particularly in response to reactive oxygen species. (J. Biol. Chem. 2003 Jun. 27; 278(26):24233-41. Epub 2003 Apr. 23).

Thus, inhibitors of 1673 can be used to ameliorate cardiovascular disease, including but not limited to atherosclerosis and coronary artery disease. 1673 polypeptides of the present invention would be useful in screening for modulators of 1673 activity.

Gene ID 54946

The human 54946 sequence (SEQ ID NO:125), known as Serine/threonine-protein kinase Sgk2, (EC 2.7.1.37) Serum/glucocorticoid regulated kinase 2, is approximately 2146 nucleotides long including untranslated regions. The coding sequence, located at about nucleic acids 220 to 1503 of SEQ ID NO:125, encodes a 427 amino acid protein (SEQ ID NO:126).

As assessed by TaqMan analysis using a human tissue panel, 54946 mRNA expression is fairly selective and is primarily expressed in human kidney, liver, pancreas, and brain.

Additional regulation studies using microarray profiling experiments or RT-PCR experiments demonstrated that i) 54946 mRNA is downregulated by 0.62 fold (p=0.001) by cholesterol in the livers of Apo E knock out mice relative to their wild-type controls; ii) 54946 mRNA is downregulated in livers of Apo E knock out mice vs. wildtype (males and females) mice; iii) 54946 mRNA is more highly expressed in the livers of males than female mice (wild type as well as knock out mice); and iv) 54946 mRNA is upregulated by 2.0 fold (p=0.008) by ciprofibrate in the livers of mice.

54946 is known as SGK2. SGK2 is a member of a large family of serine/threonine kinases. Its closest members are SGK1 and SGK3. SGK1 regulates the plasma membrane levels of the sodium channel ENaC in the kidney. 54946 falls into the subfamily which contains AKT (also known as protein kinase B) which is involved in glucose transport, glycogen synthesis, DNA synthesis, anti-apoptotic activity, and cell proliferation. The SGKs appear to have significant impact on the plasma membrane and its components. Despite its name, SGK2, unlike SGK1 is not apparently regulated by serum or glucocorticoids. Ypk1 may be the yeast homolog of SKG2 but is equally similar to SGK1. Ypk1 regulates translation initiation in response to nutrient signals, either through the TOR pathway or in a functionally related pathway parallel to TOR (Genetics. 2002 August; 161(4):1453-64). Ypk1 acts downstream of the Pkh kinases to control endocytosis by phosphorylating components of the endocytic machinery (J Cell Biol 2002 Jan. 21;156(2):241-8), which supports the notion that this family of kinases affects proteins on the plasma membrane. There are two splice variants which exist for SGK2, namely SGK2-alpha and -beta. SGK2-alpha encodes a protein of 367 amino acids (41 kD). SGK2-beta has an alternative 5′ end and encodes a protein of 427 amino acids (48 kD). SGK2 shares about 80% sequence identity with SGK1 and SGK3 within the central catalytic domain. Both the N and C termini share much less similarity. Kobayashi et al. (Biochem J. 1999, 344 Pt 1: 189-97) found that PKD 1 phosphorylated and activated SGK2-alpha in vitro.

In analogy with SGK1, SGK2 probably plays a role in regulating plasma-membrane proteins. SGK2 likely phosphorylates a ligand expressed in similar tissues which plays a role in stabilizing membrane proteins and which plays a role in regulating cholesterol. The one ligand that fits all these criteria is PDZK1. This protein stabilizes SR-BI on the plasma membrane of hepatocytes, thereby keeping its levels high. SGK2 phosphorylation of PDZK1 would inhibit is interaction with SR-BI (similar to the role SGK1 plays in inhibiting the interaction of Nedd4-2 with ENaC). Expression of SGK2 leads to increased expression of Na transporters on the apical membrane, thereby diminishing the sodium gradient. Since the sodium gradient drives bile transport, reduced bile flow occurs and cholesterol accumulates. Thus inhibiting SGK2 will increase SR-BI levels in the liver and therefore increase reverse cholesterol transport. Also, inhibition of SGK2 should lead to increased bile flow and a concomitant decrease in total plasma cholesterol. This would decrease plasma cholesterol levels.

Thus, inhibitors of 54946 can be used to ameliorate cardiovascular disease, including but not limited to atherosclerosis. 54946 polypeptides of the present invention would be useful in screening for modulators of 54946 activity.

Gene ID 2419

The human 2419 sequence (SEQ ID NO:127), known as human Chemokine receptor-like 1 (CML1) or G-protein coupled receptor DEZ or ChemR23, is approximately 1900 nucleotides long including untranslated regions. The coding sequence, located at about nucleic acids 514 to 1629 of SEQ ID NO:127, encodes a 371 amino acid protein (SEQ ID NO:128).

As assessed by TaqMan analysis using a human tissue panel, 2419 mRNA is expressed in a variety of human tissues, with the highest expression seen in arteries and veins, heart, colon and breast tumors, skin and CNS structures. Additionally, 2419 mRNA is expressed in cultured endothelial cells and primary osteoblasts. In rats treated with antihypertensive agents for three or twenty-eight days, expression of 2419 is subtly up-regulated in aortas compared to vehicle-treated controls. In spontaneously hypertensive rats (SHR), there is a significant (t-test, p<0.05) up-regulation of 2419 mRNA in aortas of the SHR and SHR stroke-prone strains at 15 weeks of age compared to Wistar Kyoto normotensive age-matched controls. In addition, the ligand chemerin is highly expressed in both rat and human vessels.

2419 is a recently de-orphaned Gi-coupled GPCR known as DEZ or ChemR23 which is activated by the ligand chemerin (Wittamer et al., J. Exp. Med., 198: 977-985 (2003); and Methner, et al., Biochem. Biophys. Res. Commun. 233: 336-342 (1997)). Chemerin is a novel protein which is encoded by the Tig-2 (tazarotene induced gene. It is secreted as a precursor with low activity; proteolytic cleavage of C-terminus produces potent, highly selective agonist of ChemR23. Chemerin is an extracellular protein which is expressed in human ascitic fluid and inflammatory samples at ng/ml concentrations. 2419 has high homology to several neuropeptide and chemoattractant receptors, such as the angiotensin II and C5a anaphylatoxin receptors. Activation of the 2419 receptor by chemerin was shown to promote calcium mobilization and chemotaxis of dendritic cells and macrophages (Wittamer et al., J. Exp. Med., 198: 977-985 (2003)). Our expression and regulation data suggest that antagonism of this GPCR will result in a beneficial effect by reducing well known vasoconstrictor signaling cascades (i.e. intracellular calcium elevations, p42/44 MAPK activation and cAMP levels). The expression of 2419 is enhanced in hypertensive states and expressed in relevant tissues involved in blood pressure maintenance (veins, arteries, kidney and skeletal muscle). Thus, inhibitors of 2419 can be used to ameliorate cardiovascular disease, including but not limited to atherosclerosis. 2419 polypeptides of the present invention would be useful in screening for modulators of 2419 activity.

Various aspects of the invention are described in further detail in the following subsections:

I. Screening Assays:

The invention provides a method (also referred to herein as a “screening assay”) for identifying modulators, i.e., candidate or test compounds or agents (e.g., peptides, peptidomimetics, small molecules (organic or inorganic) or other drugs) which bind to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins, have a stimulatory or inhibitory effect on, for example, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity, or have a stimulatory or inhibitory effect on, for example, the expression or activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate. Compounds identified using the assays described herein may be useful for treating cardiovascular diseases, e.g., atherosclerosis and/or thrombosis.

These assays are designed to identify compounds that bind to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, bind to other intracellular or extracellular proteins that interact with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, and interfere with the interaction of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein with other intercellular or extracellular proteins. For example, in the case of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, which is a transmembrane receptor-type protein, such techniques can identify ligands for such a receptor. A 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein ligand or substrate can, for example, be used to ameliorate cardiovascular diseases, e.g., atherosclerosis, ischemia/reperfusion, hypertension, restenosis, arterial inflammation, thrombosis and endothelial cell disorders. Such compounds may include, but are not limited to peptides, antibodies, or small organic or inorganic compounds. Such compounds may also include other cellular proteins.

Compounds identified via assays such as those described herein may be useful, for example, for ameliorating cardiovascular disease, e.g., athersclerosis and/or thrombosis. In instances whereby a cardiovascular disease condition results from an overall lower level of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression and/or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein in a cell or tissue, compounds that interact with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein may include compounds which accentuate or amplify the activity of the bound 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Such compounds would bring about an effective increase in the level of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein activity, thus ameliorating symptoms.

In other instances, mutations within the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene may cause aberrant types or excessive amounts of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins to be made which have a deleterious effect that leads to a cardiovascular disease. Similarly, physiological conditions may cause an excessive increase in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression leading to a cardiovascular disease. In such cases, compounds that bind to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein may be identified that inhibit the activity of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Assays for testing the effectiveness of compounds identified by techniques such as those described in this section are discussed herein.

In one embodiment, the invention provides assays for screening candidate or test compounds which are substrates of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or polypeptide or biologically active portion thereof. In another embodiment, the invention provides assays for screening candidate or test compounds which bind to or modulate the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K.S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.).

In one embodiment, an assay is a cell-based assay in which a cell which expresses a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or biologically active portion thereof is contacted with a test compound and the ability of the test compound to modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is determined. Determining the ability of the test compound to modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity can be accomplished by monitoring, for example, intracellular calcium, IP₃, cAMP, or diacylglycerol concentration, the phosphorylation profile of intracellular proteins, cell proliferation and/or migration, gene expression of, for example, cell surface adhesion molecules or genes associated with angiogenesis, or the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 regulated transcription factor. The cell can be of mammalian origin, e.g., an endothelial cell. In one embodiment, compounds that interact with a receptor domain can be screened for their ability to function as ligands, i.e., to bind to the receptor and modulate a signal transduction pathway. Identification of ligands, and measuring the activity of the ligand-receptor complex, leads to the identification of modulators (e.g., antagonists) of this interaction. Such modulators may be useful in the treatment of cardiovascular disease.

The ability of the test compound to modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 binding to a substrate or to bind to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can also be determined. Determining the ability of the test compound to modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 binding to a substrate can be accomplished, for example, by coupling the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate with a radioisotope or enzymatic label such that binding of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be determined by detecting the labeled 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate in a complex. 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 could also be coupled with a radioisotope or enzymatic label to monitor the ability of a test compound to modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 binding to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate in a complex. Determining the ability of the test compound to bind 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be accomplished, for example, by coupling the compound with a radioisotope or enzymatic label such that binding of the compound to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be determined by detecting the labeled 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 compound in a complex. For example, compounds (e.g., 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 ligands or substrates) can be labeled with ¹²⁵I, ³⁵S, ¹⁴C, or ³H, either directly or indirectly, and the radioisotope detected by direct counting of radioemmission or by scintillation counting. Compounds can further be enzymatically labeled with, for example, horseradish peroxidase, alkaline phosphatase, or luciferase, and the enzymatic label detected by determination of conversion of an appropriate substrate to product.

It is also within the scope of this invention to determine the ability of a compound (e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 ligand or substrate) to interact with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 without the labeling of any of the interactants. For example, a microphysiometer can be used to detect the interaction of a compound with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 without the labeling of either the compound or the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 (McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between a compound and 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419.

In another embodiment, an assay is a cell-based assay comprising contacting a cell expressing a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule (e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate) with a test compound and determining the ability of the test compound to modulate (e.g., stimulate or inhibit) the activity of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule. Determining the ability of the test compound to modulate the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule can be accomplished, for example, by determining the ability of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to bind to or interact with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule.

Determining the ability of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or a biologically active fragment thereof, to bind to or interact with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule can be accomplished by one of the methods described above for determining direct binding. In a preferred embodiment, determining the ability of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to bind to or interact with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule can be accomplished by determining the activity of the target molecule. For example, the activity of the target molecule can be determined by detecting induction of a cellular second 2+messenger of the target (i.e., intracellular Ca²⁺, diacylglycerol, IP₃, cAMP), detecting catalytic/enzymatic activity of the target on an appropriate substrate, detecting the induction of a reporter gene (comprising a target-responsive regulatory element operatively linked to a nucleic acid encoding a detectable marker, e.g., luciferase), or detecting a target-regulated cellular response (e.g., gene expression).

In yet another embodiment, an assay of the present invention is a cell-free assay in which a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or biologically active portion thereof, is contacted with a test compound and the ability of the test compound to bind to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or biologically active portion thereof is determined. Preferred biologically active portions of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins to be used in assays of the present invention include fragments which participate in interactions with non-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 molecules, e.g., fragments with high surface probability scores. Binding of the test compound to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be determined either directly or indirectly as described above. In a preferred embodiment, the assay includes contacting the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or biologically active portion thereof with a known compound which binds 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 to form an assay mixture, contacting the assay mixture with a test compound, and determining the ability of the test compound to interact with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, wherein determining the ability of the test compound to interact with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein comprises determining the ability of the test compound to preferentially bind to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 or biologically active portion thereof as compared to the known compound. Compounds that modulate the interaction of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 with a known target protein may be useful in regulating the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, especially a mutant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein.

In another embodiment, the assay is a cell-free assay in which a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or biologically active portion thereof is contacted with a test compound and the ability of the test compound to modulate (e.g., stimulate or inhibit) the activity of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or biologically active portion thereof is determined. Determining the ability of the test compound to modulate the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be accomplished, for example, by determining the ability of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to bind to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule by one of the methods described above for determining direct binding. Determining the ability of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to bind to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule can also be accomplished using a technology such as real-time Biomolecular Interaction Analysis (BIA) (Sjolander, S. and Urbaniczky, C. (1991) Anal. Chem. 63:2338-2345 and Szabo et al. (1995) Curr. Opin. Struct. Biol. 5:699-705). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules. In another embodiement, determining the ability of the test compound to bind to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule can also be accomplished using an immunoassay, a competition binding assay or a yeast-hybrid assay.

In another embodiment, determining the ability of the test compound to modulate the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be accomplished by determining the ability of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to further modulate the activity of a downstream effector of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule. For example, the activity of the effector molecule on an appropriate target can be determined or the binding of the effector to an appropriate target can be determined as previously described.

In yet another embodiment, the cell-free assay involves contacting a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or biologically active portion thereof with a known compound which binds the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to form an assay mixture, contacting the assay mixture with a test compound, and determining the ability of the test compound to interact with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, wherein determining the ability of the test compound to interact with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein comprises determining the ability of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to preferentially bind to or modulate the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule.

In more than one embodiment of the above assay methods of the present invention, it may be desirable to immobilize either 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 or its target molecule to facilitate separation of complexed from uncomplexed forms of one or both of the proteins, as well as to accommodate automation of the assay. Binding of a test compound to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, or interaction of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase/1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound or the test compound and either the non-adsorbed target protein or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, and the mixture incubated under conditions conducive to complex formation (e.g., at physiological conditions for salt and pH). Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 binding or activity determined using standard techniques.

Other techniques for immobilizing proteins on matrices can also be used in the screening assays of the invention. For example, either a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 target molecule can be immobilized utilizing conjugation of biotin and streptavidin. Biotinylated 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or target molecules can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). Alternatively, antibodies reactive with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or target molecules but which do not interfere with binding of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to its target molecule can be derivatized to the wells of the plate, and unbound target or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein trapped in the wells by antibody conjugation. Methods for detecting such complexes, in addition to those described above for the GST-immobilized complexes, include immunodetection of complexes using antibodies reactive with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or target molecule, as well as enzyme-linked assays which rely on detecting an enzymatic activity associated with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or target molecule.

In another embodiment, modulators of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression are identified in a method wherein a cell is contacted with a candidate compound and the expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein in the cell is determined. The level of expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein in the presence of the candidate compound is compared to the level of expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein in the absence of the candidate compound. The candidate compound can then be identified as a modulator of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression based on this comparison. For example, when expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein is greater (statistically significantly greater) in the presence of the candidate compound than in its absence, the candidate compound is identified as a stimulator of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein expression. Alternatively, when expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein is less (statistically significantly less) in the presence of the candidate compound than in its absence, the candidate compound is identified as an inhibitor of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein expression. The level of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein expression in the cells can be determined by methods described herein for detecting 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or protein.

In yet another aspect of the invention, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419(“1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-binding proteins” or “1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-bp”) and are involved in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. Such 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-binding proteins are also likely to be involved in the propagation of signals by the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 targets as, for example, downstream elements of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-mediated signaling pathway. Alternatively, such 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-binding proteins are likely to be 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 inhibitors.

The two-hybrid system is based on the modular nature of most transcription factors, which consist of separable DNA-binding and activation domains. Briefly, the assay utilizes two different DNA constructs. In one construct, the gene that codes for a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein.

In another aspect, the invention pertains to a combination of two or more of the assays described herein. For example, a modulating agent can be identified using a cell-based or a cell free assay, and the ability of the agent to modulate the activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be confirmed in vivo, e.g., in an animal such as an animal model for cardiovascular disease, e.g., atherosclerosis and/or thrombosis, as described herein.

This invention further pertains to novel agents identified by the above-described screening assays. Accordingly, it is within the scope of this invention to further use an agent identified as described herein in an appropriate animal model. For example, an agent identified as described herein (e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulating agent, an antisense 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecule, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-specific antibody, or a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

Any of the compounds, including but not limited to compounds such as those identified in the foregoing assay systems, may be tested for the ability to treat cardiovascular disease symptoms. Cell-based and animal model-based assays for the identification of compounds exhibiting such an ability to ameliorate cardiovascular disease systems are described herein.

In one aspect, cell-based systems, as described herein, may be used to identify compounds which may act to treat at least one cardiovascular disease symptom. For example, such cell systems may be exposed to a compound, suspected of exhibiting an ability to treat cardiovascular disease symptoms, at a sufficient concentration and for a time sufficient to elicit such an amelioration of cardiovascular disease symptoms in the exposed cells. After exposure, the cells are examined to determine whether one or more of the cardiovascular disease cellular phenotypes has been altered to resemble a more normal or more wild type, non-cardiovascular disease phenotype. Cellular phenotypes that are associated with cardiovascular disease states include aberrant proliferation and migration, angiogenesis, deposition of extracellular matrix components, accumulation of intracellular lipids, and expression of growth factors, cytokines, and other inflammatory mediators.

In addition, animal-based cardiovascular disease systems, such as those described herein, may be used to identify compounds capable of ameliorating cardiovascular disease symptoms. Such animal models may be used as test substrates for the identification of drugs, pharmaceuticals, therapies, and interventions which may be effective in treating cardiovascular disease. For example, animal models may be exposed to a compound, suspected of exhibiting an ability to ameliorate cardiovascular disease symptoms, at a sufficient concentration and for a time sufficient to elicit such an amelioration of cardiovascular disease symptoms in the exposed animals. The response of the animals to the exposure may be monitored by assessing the reversal of disorders associated with cardiovascular disease, for example, by counting the number of atherosclerotic plaques and/or measuring their size before and after treatment.

With regard to intervention, any treatments which reverse any aspect of cardiovascular disease symptoms should be considered as candidates for human cardiovascular disease therapeutic intervention. Dosages of test agents may be determined by deriving dose-response curves.

Additionally, gene expression patterns may be utilized to assess the ability of a compound to ameliorate cardiovascular disease symptoms. For example, the expression pattern of one or more genes may form part of a “gene expression profile” or “transcriptional profile” which may be then be used in such an assessment. “Gene expression profile” or “transcriptional profile”, as used herein, includes the pattern of mRNA expression obtained for a given tissue or cell type under a given set of conditions. Such conditions may include, but are not limited to, atherosclerosis, ischemia/reperfusion, hypertension, restenosis, and arterial inflammation, including any of the control or experimental conditions described herein, for example, atherogenic cytokine stimulation of macrophages. Gene expression profiles may be generated, for example, by utilizing a differential display procedure, Northern analysis and/or RT-PCR. In one embodiment, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences may be used as probes and/or PCR primers for the generation and corroboration of such gene expression profiles.

Gene expression profiles may be characterized for known states, either cardiovascular disease or normal, within the cell- and/or animal-based model systems. Subsequently, these known gene expression profiles may be compared to ascertain the effect a test compound has to modify such gene expression profiles, and to cause the profile to more closely resemble that of a more desirable profile.

For example, administration of a compound may cause the gene expression profile of a cardiovascular disease model system to more closely resemble the control system. Administration of a compound may, alternatively, cause the gene expression profile of a control system to begin to mimic a cardiovascular disease state. Such a compound may, for example, be used in further characterizing the compound of interest, or may be used in the generation of additional animal models.

II. Cell- and Animal-Based Model Systems

Described herein are cell- and animal-based systems which act as models for cardiovascular disease. These systems may be used in a variety of applications. For example, the cell- and animal-based model systems may be used to further characterize differentially expressed genes associated with cardiovascular disease, e.g., 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419. In addition, animal- and cell-based assays may be used as part of screening strategies designed to identify compounds which are capable of ameliorating cardiovascular disease symptoms, as described, below. Thus, the animal- and cell-based models may be used to identify drugs, pharmaceuticals, therapies and interventions which may be effective in treating cardiovascular disease. Furthermore, such animal models may be used to determine the LD50 and the ED50 in animal subjects, and such data can be used to determine the in vivo efficacy of potential cardiovascular disease treatments.

A. Animal-Based Systems

Animal-based model systems of cardiovascular disease may include, but are not limited to, non-recombinant and engineered transgenic animals.

Non-recombinant animal models for cardiovascular disease may include, for example, genetic models. Such genetic cardiovascular disease models may include, for example, ApoB or ApoR deficient pigs (Rapacz, et al., 1986, Science 234:1573-1577) and Watanabe heritable hyperlipidemic (WHHL) rabbits (Kita et al., 1987, Proc. Natl. Acad. Sci USA 84: 5928-5931). Transgenic mouse models in cardiovascular disease and angiogenesis are reviewed in Carmeliet, P. and Collen, D. (2000) J. Pathol. 190:387-405.

Non-recombinant, non-genetic animal models of atherosclerosis may include, for example, pig, rabbit, or rat models in which the animal has been exposed to either chemical wounding through dietary supplementation of LDL, or mechanical wounding through balloon catheter angioplasty. Animal models of cardiovascular disease also include rat myocardial infarction models (described in, for example, Schwarz, ER et al. (2000) J. Am. Coll. Cardiol. 35:1323-1330) and models of chromic cardiac ischemia in rabbits (described in, for example, Operschall, C et al. (2000) J. Appl. Physiol. 88:1438-1445).

Additionally, animal models exhibiting cardiovascular disease symptoms may be engineered by using, for example, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences described above, in conjunction with techniques for producing transgenic animals that are well known to those of skill in the art. For example, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences may be introduced into, and overexpressed in, the genome of the animal of interest, or, if endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences are present, they may either be overexpressed or, alternatively, be disrupted in order to underexpress or inactivate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression, such as described for the disruption of ApoE in mice (Plump et al., 1992, Cell 71: 343-353).

The host cells of the invention can also be used to produce non-human transgenic animals. For example, in one embodiment, a host cell of the invention is a fertilized oocyte or an embryonic stem cell into which 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-coding sequences have been introduced. Such host cells can then be used to create non-human transgenic animals in which exogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequences have been introduced into their genome or homologous recombinant animals in which endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequences have been altered. Such animals are useful for studying the function and/or activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 and for identifying and/or evaluating modulators of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, and the like. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene has been altered by homologous recombination between the endogenous gene and an exogenous DNA molecule introduced into a cell of the animal, e.g., an embryonic cell of the animal, prior to development of the animal.

A transgenic animal used in the methods of the invention can be created by introducing a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-encoding nucleic acid into the male pronuclei of a fertilized oocyte, e.g., by microinjection, retroviral infection, and allowing the oocyte to develop in a pseudopregnant female foster animal. The 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 cDNA sequence can be introduced as a transgene into the genome of a non-human animal. Alternatively, a nonhuman homologue of a human 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, such as a mouse or rat 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, can be used as a transgene. Alternatively, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene homologue, such as another 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 family member, can be isolated based on hybridization to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 cDNA sequences and used as a transgene. Intronic sequences and polyadenylation signals can also be included in the transgene to increase the efficiency of expression of the transgene. A tissue-specific regulatory sequence(s) can be operably linked to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 transgene to direct expression of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to particular cells. Methods for generating transgenic animals via embryo manipulation and microinjection, particularly animals such as mice, have become conventional in the art and are described, for example, in U.S. Pat. Nos. 4,736,866 and 4, 870, 009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 transgene in its genome and/or expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA in tissues or cells of the animals. A transgenic founder animal can then be used to breed additional animals carrying the transgene. Moreover, transgenic animals carrying a transgene encoding a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can further be bred to other transgenic animals carrying other transgenes.

To create a homologous recombinant animal, a vector is prepared which contains at least a portion of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene into which a deletion, addition or substitution has been introduced to thereby alter, e.g., functionally disrupt, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene. The 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene can be a human gene but more preferably, is a non-human homologue of a human 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene. For example, a rat 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene can be used to construct a homologous recombination nucleic acid molecule, e.g., a vector, suitable for altering an endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene in the mouse genome. In a preferred embodiment, the homologous recombination nucleic acid molecule is designed such that, upon homologous recombination, the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene is functionally disrupted (i.e., no longer encodes a functional protein; also referred to as a “knock out” vector). Alternatively, the homologous recombination nucleic acid molecule can be designed such that, upon homologous recombination, the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene is mutated or otherwise altered but still encodes functional protein (e.g., the upstream regulatory region can be altered to thereby alter the expression of the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein). In the homologous recombination nucleic acid molecule, the altered portion of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene is flanked at its 5′ and 3′ ends by additional nucleic acid sequence of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene to allow for homologous recombination to occur between the exogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene carried by the homologous recombination nucleic acid molecule and an endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene in a cell, e.g., an embryonic stem cell. The additional flanking 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid sequence is of sufficient length for successful homologous recombination with the endogenous gene. Typically, several kilobases of flanking DNA (both at the 5′ and 3′ ends) are included in the homologous recombination nucleic acid molecule (see, e.g., Thomas, K. R. and Capecchi, M. R. (1987) Cell 51:503 for a description of homologous recombination vectors). The homologous recombination nucleic acid molecule is introduced into a cell, e.g., an embryonic stem cell line (e.g., by electroporation) and cells in which the introduced 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene has homologously recombined with the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene are selected (see e.g., Li, E. et al. (1992) Cell 69:915). The selected cells can then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination nucleic acid molecules, e.g., vectors, or homologous recombinant animals are described further in Bradley, A. (1991) Current Opinion in Biotechnology 2:823-829 and in PCT International Publication Nos.: WO 90/11354 by Le Mouellec et al.; WO 91/01140 by Smithies et al.; WO 92/0968 by Zijlstra et al.; and WO 93/04169 by Berns et al.

In another embodiment, transgenic non-human animals for use in the methods of the invention can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, I. et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

The 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 transgenic animals that express 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 peptide (detected immunocytochemically, using antibodies directed against 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 epitopes) at easily detectable levels should then be further evaluated to identify those animals which display characteristic cardiovascular disease symptoms. Such cardiovascular disease symptoms may include, for example, increased prevalence and size of fatty streaks and/or cardiovascular disease plaques.

Additionally, specific cell types (e.g., endothelial cells) within the transgenic animals may be analyzed and assayed for cellular phenotypes characteristic of cardiovascular disease. In the case of endothelial cells, such phenotypes include, but are not limited to cell proliferation, migration, angiogenesis, production of proinflammatory growth factors and cytokines, and adhesion to inflammatory cells. In the case of monocytes, such phenotypes may include but are not limited to increases in rates of LDL uptake, adhesion to endothelial cells, transmigration, foam cell formation, fatty streak formation, and production of foam cell specific products. Cellular phenotypes may include a particular cell type's pattern of expression of genes associated with cardiovascular disease as compared to known expression profiles of the particular cell type in animals exhibiting cardiovascular disease symptoms.

B. Cell-Based Systems

Cells that contain and express 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences which encode a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, and, further, exhibit cellular phenotypes associated with cardiovascular disease, may be used to identify compounds that exhibit anti-cardiovascular disease activity. Such cells may include non-recombinant monocyte cell lines, such as U937 (ATCC# CRL-1593), THP-1 (ATCC#TIB-202), and P388D1 (ATCC# TIB-63); endothelial cells such as human umbilical vein endothelial cells (HUVECs), human microvascular endothelial cells (HMVEC), and bovine aortic endothelial cells (BAECs); as well as generic mammalian cell lines such as HeLa cells and COS cells, e.g., COS-7 (ATCC# CRL-1651). Further, such cells may include recombinant, transgenic cell lines. For example, the cardiovascular disease animal models of the invention, discussed above, may be used to generate cell lines, containing one or more cell types involved in cardiovascular disease, that can be used as cell culture models for this disorder. While primary cultures derived from the cardiovascular disease transgenic animals of the invention may be utilized, the generation of continuous cell lines is preferred. For examples of techniques which may be used to derive a continuous cell line from the transgenic animals, see Small et al., (1985) Mol. Cell Biol. 5:642-648.

Alternatively, cells of a cell type known to be involved in cardiovascular disease may be transfected with sequences capable of increasing or decreasing the amount of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression within the cell. For example, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences may be introduced into, and overexpressed in, the genome of the cell of interest, or, if endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences are present, they may be either overexpressed or, alternatively disrupted in order to underexpress or inactivate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression.

In order to overexpress a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, the coding portion of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene may be ligated to a regulatory sequence which is capable of driving gene expression in the cell type of interest, e.g., an endothelial cell. Such regulatory regions will be well known to those of skill in the art, and may be utilized in the absence of undue experimentation. Recombinant methods for expressing target genes are described above.

For underexpression of an endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequence, such a sequence may be isolated and engineered such that when reintroduced into the genome of the cell type of interest, the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 alleles will be inactivated. Preferably, the engineered 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequence is introduced via gene targeting such that the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequence is disrupted upon integration of the engineered 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequence into the cell's genome. Transfection of host cells with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 genes is discussed, above.

Cells treated with compounds or transfected with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 genes can be examined for phenotypes associated with cardiovascular disease. In the case of monocytes, such phenotypes include but are not limited to increases in rates of LDL uptake, adhesion to endothelial cells, transmigration, foam cell formation, fatty streak formation, and production by foam cells of growth factors such as bFGF, IGF-I, VEGF, IL-1, M-CSF, TGFβ, TGFα, TNFα, HB-EGF, PDGF, IFN-γ, and GM-CSF. Transmigration rates, for example, may be measured using the in vitro system of Navab et al. (1988) J. Clin. Invest. 82:1853-1863, by quantifying the number of monocytes that migrate across the endothelial monolayer and into the collagen layer of the subendothelial space.

Similarly, endothelial cells can be treated with test compounds or transfected with genetically engineered 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 genes. The endothelial cells can then be examined for phenotypes associated with cardiovascular disease, including, but not limited to changes in cellular morphology, cell proliferation, cell migration, and mononuclear cell adhesion; or for the effects on production of other proteins involved in cardiovascular disease such as adhesion molecules (e.g., ICAM, VCAM, E-selectin), growth factors and cytokines (e.g., PDGF, IL-1β, TNFα, MCF), and proteins involved in angiogenesis (e.g., FLK, FLT).

Transfection of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid may be accomplished by using standard techniques (described in, for example, Ausubel (1989) supra). Transfected cells should be evaluated for the presence of the recombinant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences, for expression and accumulation of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA, and for the presence of recombinant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein production. In instances wherein a decrease in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression is desired, standard techniques may be used to demonstrate whether a decrease in endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression and/or in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein production is achieved.

Cellular models for the study of cardiovascular disease and angiogenesis include models of endothelial cell differentiation on Matrigel (Baatout, S. et al. (1996) Rom. J. Intern. Med. 34:263-269; Benelli, R et al. (1999) Int. J. Biol. Markers 14:243-246), embryonic stem cell models of vascular morphogenesis (Doetschman, T. et al. (1993) Hypertension 22:618-629), the culture of microvessel fragments in physiological gels (Hoying, J B et al. (1996) In Vitro Cell Dev. Biol. Anim. 32: 409-419; U.S. Pat. No. 5,976,782), and the treatment of endothelial cells and smooth muscle cells with atherogenic and angiogenic factors including growth factors and cytokines (e.g., IL-1β, PDGF, TNFα, VEGF), homocysteine, and LDL. In vitro angiogenesis models are described in, for example, Black, AF et al. (1999) Cell Biol. Toxicol. 15:81-90.

Also provided are cells or a purified preparation thereof, e.g., human cells, in which an endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 is under the control of a regulatory sequence that does not normally control the expression of the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene. The expression characteristics of an endogenous gene within a cell, e.g., a cell line or microorganism, can be modified by inserting a heterologous DNA regulatory element into the genome of the cell such that the inserted regulatory element is operably linked to the endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene. For example, an endogenous 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, e.g., a gene which is “transcriptionally silent,” e.g., not normally expressed, or expressed only at very low levels, may be activated by inserting a regulatory element which is capable of promoting the expression of a normally expressed gene product in that cell. Techniques such as targeted homologous recombinations, can be used to insert the heterologous DNA as described in, e.g., Chappel, U.S. Pat. No. 5,272,071; WO 91/06667, published on May 16, 1991.

III. Predictive Medicine:

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein and/or nucleic acid expression as well as 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity, in the context of a biological sample (e.g., blood, serum, cells, e.g., endothelial cells, or tissue, e.g., vascular tissue) to thereby determine whether an individual is afflicted with a cardiovascular disease. The invention also provides for prognostic (or predictive) assays for determining whether an individual is at risk of developing a cardiovascular disorder. For example, mutations in a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene can be assayed for in a biological sample. Such assays can be used for prognostic or predictive purpose to thereby phophylactically treat an individual prior to the onset of a cardiovascular disorder, e.g., atherosclerosis.

Another aspect of the invention pertains to monitoring the influence of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulators (e.g., anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 ribozymes) on the expression or activity of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 in clinical trials.

These and other agents are described in further detail in the following sections.

A. Diagnostic Assays For Cardiovascular Disease

To determine whether a subject is afflicted with a cardiovascular disease, a biological sample may be obtained from a subject and the biological sample may be contacted with a compound or an agent capable of detecting a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or nucleic acid (e.g., mRNA or genomic DNA) that encodes a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, in the biological sample. A preferred agent for detecting 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or genomic DNA is a labeled nucleic acid probe capable of hybridizing to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or genomic DNA. The nucleic acid probe can be, for example, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid set forth in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 or a portion thereof, such as an oligonucleotide of at least 15, 20, 25, 30, 25, 40, 45, 50, 100, 250 or 500 nucleotides in length and sufficient to specifically hybridize under stringent conditions to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA or genomic DNA. Other suitable probes for use in the diagnostic assays of the invention are described herein.

A preferred agent for detecting 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein in a sample is an antibody capable of binding to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells, and biological fluids isolated from a subject, as well as tissues, cells, and fluids present within a subject. That is, the detection method of the invention can be used to detect 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA, protein, or genomic DNA in a biological sample in vitro as well as in vivo. For example, in vitro techniques for detection of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA include Northern hybridizations and in situ hybridizations. In vitro techniques for detection of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein include enzyme linked immunosorbent assays (ELISAs), Western blots, immunoprecipitations and immunofluorescence. In vitro techniques for detection of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 genomic DNA include Southern hybridizations. Furthermore, in vivo techniques for detection of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein include introducing into a subject a labeled anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody. For example, the antibody can be labeled with a radioactive marker whose presence and location in a subject can be detected by standard imaging techniques.

In another embodiment, the methods further involve obtaining a control biological sample from a control subject, contacting the control sample with a compound or agent capable of detecting 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA, or genomic DNA, such that the presence of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA or genomic DNA is detected in the biological sample, and comparing the presence of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA or genomic DNA in the control sample with the presence of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA or genomic DNA in the test sample.

B. Prognostic Assays For Cardiovascular Disease

The present invention further pertains to methods for identifying subjects having or at risk of developing a cardiovascular disease associated with aberrant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity.

As used herein, the term “aberrant” includes a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity which deviates from the wild type 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity. Aberrant expression or activity includes increased or decreased expression or activity, as well as expression or activity which does not follow the wild type developmental pattern of expression or the subcellular pattern of expression. For example, aberrant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity is intended to include the cases in which a mutation in the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene causes the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene to be under-expressed or over-expressed and situations in which such mutations result in a non-functional 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or a protein which does not function in a wild-type fashion, e.g., a protein which does not interact with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate, or one which interacts with a non-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate.

The assays described herein, such as the preceding diagnostic assays or the following assays, can be used to identify a subject having or at risk of developing a cardiovascular disease, e.g., including but not limited to, atherosclerosis, ischemia/reperfusion injury, hypertension, restenosis, arterial inflammation, and endothelial cell disorders. A biological sample may be obtained from a subject and tested for the presence or absence of a genetic alteration. For example, such genetic alterations can be detected by ascertaining the existence of at least one of 1) a deletion of one or more nucleotides from a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, 2) an addition of one or more nucleotides to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, 3) a substitution of one or more nucleotides of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, 4) a chromosomal rearrangement of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, 5) an alteration in the level of a messenger RNA transcript of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, 6) aberrant modification of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, such as of the methylation pattern of the genomic DNA, 7) the presence of a non-wild type splicing pattern of a messenger RNA transcript of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, 8) a non-wild type level of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-protein, 9) allelic loss of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, and 10) inappropriate post-translational modification of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-protein.

As described herein, there are a large number of assays known in the art which can be used for detecting genetic alterations in a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene. For example, a genetic alteration in a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene may be detected using a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene (see Abravaya et al. (1995) Nucleic Acids Res. 23:675-682). This method includes collecting a biological sample from a subject, isolating nucleic acid (e.g., genomic DNA, mRNA or both) from the sample, contacting the nucleic acid sample with one or more primers which specifically hybridize to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene under conditions such that hybridization and amplification of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene from a biological sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be identified by hybridizing biological sample derived and control nucleic acids, e.g., DNA or RNA, to high density arrays containing hundreds or thousands of oligonucleotide probes (Cronin, M. T. et al. (1996) Human Mutation 7:244-255; Kozal, M. J. et al. (1996) Nature Medicine 2:753-759). For example, genetic mutations in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be identified in two dimensional arrays containing light-generated DNA probes as described in Cronin, M. T. et al. (1996) supra. Briefly, a first hybridization array of probes can be used to scan through long stretches of DNA in a sample and control to identify base changes between the sequences by making linear arrays of sequential, overlapping probes. This step allows for the identification of point mutations. This step is followed by a second hybridization array that allows for the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene in a biological sample and detect mutations by comparing the sequence of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 in the biological sample with the corresponding wild-type (control) sequence. Examples of sequencing reactions include those based on techniques developed by Maxam and Gilbert (1977) Proc. Natl. Acad. Sci. USA 74:560) or Sanger (1977) Proc. Natl. Acad. Sci. USA 74:5463). It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad Sci USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequence, e.g., a wild-type 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 genes. For example, single strand conformation polymorphism (SSCP) may be used to detect differences in electrophoretic mobility between mutant and wild type nucleic acids (Orita et al. (1989) Proc Natl. Acad. Sci USA: 86:2766; see also Cotton (1993) Mutat. Res. 285:125-144 and Hayashi (1992) Genet. Anal. Tech. Appl. 9:73-79). Single-stranded DNA fragments of sample and control 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Furthermore, the prognostic assays described herein can be used to determine whether a subject can be administered a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator (e.g., an agonist, antagonist, peptidomimetic, protein, peptide, nucleic acid, or small molecule) to effectively treat a cardiovascular disease, e.g., atherosclerosis.

C. Monitoring of Effects During Clinical Trials

The present invention further provides methods for determining the effectiveness of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator (e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator identified herein) in treating a cardiovascular disease, e.g., atherosclerosis and/or thrombosis, in a subject. For example, the effectiveness of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator in increasing 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression, protein levels, or in upregulating 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity, can be monitored in clinical trials of subjects exhibiting decreased 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression, protein levels, or downregulated 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. Alternatively, the effectiveness of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator in decreasing 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression, protein levels, or in downregulating 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity, can be monitored in clinical trials of subjects exhibiting increased 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression, protein levels, or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. In such clinical trials, the expression or activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, and preferably, other genes that have been implicated in, for example, atherosclerosis and/or thrombosis can be used as a “read out” or marker of the phenotype of a particular cell, e.g., a vascular endothelial cell.

For example, and not by way of limitation, genes, including 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419, that are modulated in cells by treatment with an agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity (e.g., identified in a screening assay as described herein) can be identified. Thus, to study the effect of agents which modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity on subjects suffering from a cardiovascular disease in, for example, a clinical trial, cells can be isolated and RNA prepared and analyzed for the levels of expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 and other genes implicated in the cardiovascular disease. The levels of gene expression (e.g., a gene expression pattern) can be quantified by Northern blot analysis or RT-PCR, as described herein, or alternatively by measuring the amount of protein produced, by one of the methods described herein, or by measuring the levels of activity of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 or other genes. In this way, the gene expression pattern can serve as a marker, indicative of the physiological response of the cells to the agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. This response state may be determined before, and at various points during treatment of the individual with the agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity.

In a preferred embodiment, the present invention provides a method for monitoring the effectiveness of treatment of a subject with an agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity (e.g., an agonist, antagonist, peptidomimetic, protein, peptide, nucleic acid, or small molecule identified by the screening assays described herein) including the steps of (i) obtaining a pre-administration sample from a subject prior to administration of the agent; (ii) detecting the level of expression of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA, or genomic DNA in the pre-administration sample; (iii) obtaining one or more post-administration samples from the subject; (iv) detecting the level of expression or activity of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA, or genomic DNA in the post-administration samples; (v) comparing the level of expression or activity of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA, or genomic DNA in the pre-administration sample with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, mRNA, or genomic DNA in the post administration sample or samples; and (vi) altering the administration of the agent to the subject accordingly. For example, increased administration of the agent may be desirable to increase the expression or activity of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 to higher levels than detected, i.e., to increase the effectiveness of the agent. Alternatively, decreased administration of the agent may be desirable to decrease expression or activity of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 to lower levels than detected, i.e. to decrease the effectiveness of the agent. According to such an embodiment, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity may be used as an indicator of the effectiveness of an agent, even in the absence of an observable phenotypic response.

IV. Methods of Treatment of Subjects Suffering From Cardiovascular Disease:

The present invention provides for both prophylactic and therapeutic methods of treating a subject, e.g., a human, at risk of (or susceptible to) a cardiovascular disease such as atherosclerosis, ischemia/reperfusion injury, hypertension, restenosis, arterial inflammation, thrombosis, and endothelial cell disorders. With regard to both prophylactic and therapeutic methods of treatment, such treatments may be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype”, or “drug response genotype”).

Thus, another aspect of the invention provides methods for tailoring an subject's prophylactic or therapeutic treatment with either the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 molecules of the present invention or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

A. Prophylactic Methods

In one aspect, the invention provides a method for preventing in a subject, a cardiovascular disease by administering to the subject an agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity, e.g., modulation of calcium influx, cellular migration, or formation of atherosclerotic lesions. Subjects at risk for a cardiovascular disease, e.g., atherosclerosis and/or thrombosis, can be identified by, for example, any or a combination of the diagnostic or prognostic assays described herein. Administration of a prophylactic agent can occur prior to the manifestation of symptoms characteristic of aberrant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity, such that a cardiovascular disease is prevented or, alternatively, delayed in its progression. Depending on the type of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 aberrancy, for example, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 agonist or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antagonist agent can be used for treating the subject. The appropriate agent can be determined based on screening assays described herein.

B. Therapeutic Methods

Described herein are methods and compositions whereby cardiovascular disease symptoms may be ameliorated. Certain cardiovascular diseases are brought about, at least in part, by an excessive level of a gene product, or by the presence of a gene product exhibiting an abnormal or excessive activity. As such, the reduction in the level and/or activity of such gene products would bring about the amelioration of cardiovascular disease symptoms. Techniques for the reduction of gene expression levels or the activity of a protein are discussed below.

Alternatively, certain other cardiovascular diseases are brought about, at least in part, by the absence or reduction of the level of gene expression, or a reduction in the level of a protein's activity. As such, an increase in the level of gene expression and/or the activity of such proteins would bring about the amelioration of cardiovascular disease symptoms.

In some cases, the up-regulation of a gene in a disease state reflects a protective role for that gene product in responding to the disease condition. Enhancement of such a gene's expression, or the activity of the gene product, will reinforce the protective effect it exerts. Some cardiovascular disease states may result from an abnormally low level of activity of such a protective gene. In these cases also, an increase in the level of gene expression and/or the activity of such gene products would bring about the amelioration of cardiovascular disease symptoms. Techniques for increasing target gene expression levels or target gene product activity levels are discussed herein.

Accordingly, another aspect of the invention pertains to methods of modulating 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity for therapeutic purposes. Accordingly, in an exemplary embodiment, the modulatory method of the invention involves contacting a cell with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 or agent that modulates one or more of the activities of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein activity associated with the cell (e.g., an endothelial cell or an ovarian cell). An agent that modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein activity can be an agent as described herein, such as a nucleic acid or a protein, a naturally-occurring target molecule of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein (e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 ligand or substrate), a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 agonist or antagonist, a peptidomimetic of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 agonist or antagonist, or other small molecule. In one embodiment, the agent stimulates one or more 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activities. Examples of such stimulatory agents include active 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein and a nucleic acid molecule encoding 1722, 10280, 59917, 85553, 10653, 9235, 21668, 17794, 2210, 6169, 10102, 21061, 17662, 1468, 12282, 6350, 9035, 1.820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 that has been introduced into the cell. In another embodiment, the agent inhibits one or more 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activities. Examples of such inhibitory agents include antisense 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecules, anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies, and 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 inhibitors. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or nucleic acid molecule. In one embodiment, the method involves administering an agent (e.g., an agent identified by a screening assay described herein), or combination of agents that modulates (e.g., upregulates or downregulates) 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity. In another embodiment, the method involves administering a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or nucleic acid molecule as therapy to compensate for reduced, aberrant, or unwanted 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity.

Stimulation of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is desirable in situations in which 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 is abnormally downregulated and/or in which increased 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is likely to have a beneficial effect. Likewise, inhibition of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is desirable in situations in which 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 is abnormally upregulated and/or in which decreased 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is likely to have a beneficial effect.

(i) Methods for Inhibiting Target Gene Expression, Synthesis, or Activity

As discussed above, genes involved in cardiovascular disorders may cause such disorders via an increased level of gene activity. In some cases, such up-regulation may have a causative or exacerbating effect on the disease state. A variety of techniques may be used to inhibit the expression, synthesis, or activity of such genes and/or proteins.

For example, compounds such as those identified through assays described above, which exhibit inhibitory activity, may be used in accordance with the invention to ameliorate cardiovascular disease symptoms. Such molecules may include, but are not limited to, small organic molecules, peptides, antibodies, and the like.

For example, compounds can be administered that compete with endogenous ligand for the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. The resulting reduction in the amount of ligand-bound 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein will modulate endothelial cell physiology. Compounds that can be particularly useful for this purpose include, for example, soluble proteins or peptides, such as peptides comprising one or more of the extracellular domains, or portions and/or analogs thereof, of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, including, for example, soluble fusion proteins such as Ig-tailed fusion proteins. (For a discussion of the production of Ig-tailed fusion proteins, see, for example, U.S. Pat. No. 5,116,964). Alternatively, compounds, such as ligand analogs or antibodies, that bind to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 receptor site, but do not activate the protein, (e.g., receptor-ligand antagonists) can be effective in inhibiting 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein activity.

Further, antisense and ribozyme molecules which inhibit expression of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene may also be used in accordance with the invention to inhibit aberrant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene activity. Still further, triple helix molecules may be utilized in inhibiting aberrant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene activity.

The antisense nucleic acid molecules used in the methods of the invention are typically administered to a subject or generated in situ such that they hybridize with or bind to cellular mRNA and/or genomic DNA encoding a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong po1 II or po1 III promoter are preferred.

In yet another embodiment, an antisense nucleic acid molecule used in the methods of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid used in the methods of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA transcripts to thereby inhibit translation of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA. A ribozyme having specificity for a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-encoding nucleic acid can be designed based upon the nucleotide sequence of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 cDNA disclosed herein (i.e., SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127). For example, a derivative of a Tetrahymena L-19 IVS RNA can be constructed in which the nucleotide sequence of the active site is complementary to the nucleotide sequence to be cleaved in a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-encoding mRNA (see, for example, Cech et al. U.S. Pat. No. 4,987,071; and Cech et al. U.S. Pat. No. 5,116,742). Alternatively, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA can be used to select a catalytic RNA having a specific ribonuclease activity from a pool of RNA molecules (see, for example, Bartel, D. and Szostak, J. W. (1993) Science 261:1411-1418).

1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression can also be inhibited by targeting nucleotide sequences complementary to the regulatory region of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 (e.g., the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 promoter and/or enhancers) to form triple helical structures that prevent transcription of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene in target cells (see, for example, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15).

Antibodies that are both specific for the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein and interfere with its activity may also be used to modulate or inhibit 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein function. Such antibodies may be generated using standard techniques described herein, against the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein itself or against peptides corresponding to portions of the protein. Such antibodies include but are not limited to polyclonal, monoclonal, Fab fragments, single chain antibodies, or chimeric antibodies.

In instances where the target gene protein is intracellular and whole antibodies are used, internalizing antibodies may be preferred. Lipofectin liposomes may be used to deliver the antibody or a fragment of the Fab region which binds to the target epitope into cells. Where fragments of the antibody are used, the smallest inhibitory fragment which binds to the target protein's binding domain is preferred. For example, peptides having an amino acid sequence corresponding to the domain of the variable region of the antibody that binds to the target gene protein may be used. Such peptides may be synthesized chemically or produced via recombinant DNA technology using methods well known in the art (described in, for example, Creighton (1983), supra; and Sambrook et al. (1989) supra). Single chain neutralizing antibodies which bind to intracellular target gene epitopes may also be administered. Such single chain antibodies may be administered, for example, by expressing nucleotide sequences encoding single-chain antibodies within the target cell population by utilizing, for example, techniques such as those described in Marasco et al. (1993) Proc. Natl. Acad. Sci. USA 90:7889-7893).

In some instances, the target gene protein is extracellular, or is a transmembrane protein, such as the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Antibodies that are specific for one or more extracellular domains of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, for example, and that interfere with its activity, are particularly useful in treating cardiovascular disease. Such antibodies are especially efficient because they can access the target domains directly from the bloodstream. Any of the administration techniques described below which are appropriate for peptide administration may be utilized to effectively administer inhibitory target gene antibodies to their site of action.

(ii) Methods for Restoring or Enhancing Target Gene Activity

Genes that cause cardiovascular disease may be underexpressed within cardiovascular disease situations. Alternatively, the activity of the protein products of such genes may be decreased, leading to the development of cardiovascular disease symptoms. Such down-regulation of gene expression or decrease of protein activity might have a causative or exacerbating effect on the disease state.

In some cases, genes that are up-regulated in the disease state might be exerting a protective effect. A variety of techniques may be used to increase the expression, synthesis, or activity of genes and/or proteins that exert a protective effect in response to cardiovascular disease conditions.

Described in this section are methods whereby the level 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity may be increased to levels wherein cardiovascular disease symptoms are ameliorated. The level of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity may be increased, for example, by either increasing the level of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene expression or by increasing the level of active 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein which is present.

For example, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, at a level sufficient to ameliorate cardiovascular disease symptoms may be administered to a patient exhibiting such symptoms. Any of the techniques discussed below may be used for such administration. One of skill in the art will readily know how to determine the concentration of effective, non-toxic doses of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, utilizing techniques such as those described below.

Additionally, RNA sequences encoding a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein may be directly administered to a patient exhibiting cardiovascular disease symptoms, at a concentration sufficient to produce a level of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein such that cardiovascular disease symptoms are ameliorated. Any of the techniques discussed below, which achieve intracellular administration of compounds, such as, for example, liposome administration, may be used for the administration of such RNA molecules. The RNA molecules may be produced, for example, by recombinant techniques such as those described herein.

Further, subjects may be treated by gene replacement therapy. One or more copies of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene, or a portion thereof, that directs the production of a normal 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 function, may be inserted into cells using vectors which include, but are not limited to adenovirus, adeno-associated virus, and retrovirus vectors, in addition to other particles that introduce DNA into cells, such as liposomes. Additionally, techniques such as those described above may be used for the introduction of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene sequences into human cells.

Cells, preferably, autologous cells, containing 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expressing gene sequences may then be introduced or reintroduced into the subject at positions which allow for the amelioration of cardiovascular disease symptoms. Such cell replacement techniques may be preferred, for example, when the gene product is a secreted, extracellular gene product.

C. Pharmaceutical Compositions

Another aspect of the invention pertains to methods for treating a subject suffering from a cardiovascular disease, e.g., atherosclerosis. These methods involve administering to a subject an agent which modulates 1722, 10280, 59917, 85553, 10653, 92-35, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity (e.g., an agent identified by a screening assay described herein), or a combination of such agents. In another embodiment, the method involves administering to a subject a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or nucleic acid molecule as therapy to compensate for reduced, aberrant, or unwanted 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 expression or activity.

Stimulation of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is desirable in situations in which 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180; 26005, 554, 16408, 42028, 112091, 13886, 13942, 1673, 54946 or 2419 is abnormally downregulated and/or in which increased 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is likely to have a beneficial effect. Likewise, inhibition of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is desirable in situations in which 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 is abnormally upregulated and/or in which decreased 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity is likely to have a beneficial effect, e.g., inhibition of atherosclerotic lesion formation.

The agents which modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity can be administered to a subject using pharmaceutical compositions suitable for such administration. Such compositions typically comprise the agent (e.g., nucleic acid molecule, protein, or antibody) and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition used in the therapeutic methods of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the agent that modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity (e.g., a fragment of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or an anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The agents that modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity can also be prepared in the form of suppositories (e.g., with conventional suppository bases such as cocoa butter and other glycerides) or retention enemas for rectal delivery.

In one embodiment, the agents that modulate 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the agent that modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity and the particular therapeutic effect to be achieved, and the limitations inherent in the art of compounding such an agent for the treatment of subjects.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50. Agents which exhibit large therapeutic indices are preferred. While agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage for use in humans. The dosage of such 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulating agents lies preferably within a range of circulating concentrations that include the ED50 with little or no toxicity. The dosage may vary within this range depending upon the dosage form employed and the route of administration utilized. For any agent used in the therapeutic methods of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Further, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982). Alternatively, an antibody can be conjugated to a second antibody to form an antibody heteroconjugate as described by Segal in U.S. Pat. No. 4,676,980.

The nucleic acid molecules used in the methods of the invention can be inserted into vectors and used as gene therapy vectors. Gene therapy vectors can be delivered to a subject by, for example, intravenous injection, local administration (see U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

D. Pharmacogenomics

In conjunction with the therapeutic methods of the invention, pharmacogenomics (i.e., the study of the relationship between a subject's genotype and that subject's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity, as well as tailoring the dosage and/or therapeutic regimen of treatment with an agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11): 983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate aminopeptidase deficiency (G6PD) is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants). Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach” can be utilized to identify genes that predict drug response. According to this method, if a gene that encodes a drug target is known (e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein used in the methods of the present invention), all common variants of that gene can be fairly easily identified in the population and it can be determined if having one version of the gene versus another is associated with a particular drug response.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and the cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Alternatively, a method termed the “gene expression profiling” can be utilized to identify genes that predict drug response. For example, the gene expression of an animal dosed with a drug (e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 molecule or 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator used in the methods of the present invention) can give an indication whether gene pathways related to toxicity have been turned on.

Information generated from more than one of the above pharmacogenomics approaches can be used to determine appropriate dosage and treatment regimens for prophylactic or therapeutic treatment of a subject. This knowledge, when applied to dosing or drug selection, can avoid adverse reactions or therapeutic failure and, thus, enhance therapeutic or prophylactic efficiency when treating a subject suffering from a cardiovascular disease, e.g., atherosclerosis, with an agent which modulates 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity.

V. Recombinant Expression Vectors and Host Cells Used in the Methods of the Invention

The methods of the invention (e.g., the screening assays described herein) include the use of vectors, preferably expression vectors, containing a nucleic acid encoding a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors to be used in the methods of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins, mutant forms of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins, fusion proteins, and the like).

The recombinant expression vectors to be used in the methods of the invention can be designed for expression of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins in prokaryotic or eukaryotic cells. For example, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins can be expressed in bacterial cells such as E. coli, insect cells (using baculovirus expression vectors), yeast cells, or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Purified fusion proteins can be utilized in 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity assays, (e.g., direct assays or competitive assays described in detail below), or to generate antibodies specific for 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins. In a preferred embodiment, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion protein expressed in a retroviral expression vector of the present invention can be utilized to infect bone marrow cells which are subsequently transplanted into irradiated recipients. The pathology of the subject recipient is then examined after sufficient time has passed (e.g., six weeks).

In another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J. et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid).

The methods of the invention may further use a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific, or cell type specific expression of antisense RNA. The antisense expression vector can be in the form of a recombinant plasmid, phagemid, or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews-Trends in Genetics, Vol. 1(1) 1986.

Another aspect of the invention pertains to the use of host cells into which a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecule of the invention is introduced, e.g., a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecule within a recombinant expression vector or a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecule containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell used in the methods of the invention, such as a prokaryotic or eukaryotic host cell in culture, can be used to produce (i.e., express) a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Accordingly, the invention further provides methods for producing a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein using the host cells of the invention. In one embodiment, the method comprises culturing the host cell of the invention (into which a recombinant expression vector encoding a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein has been introduced) in a suitable medium such that a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein is produced. In another embodiment, the method further comprises isolating a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein from the medium or the host cell.

VI. Isolated Nucleic Acid Molecules Used in the Methods of the Invention

The methods of the invention include the use of isolated nucleic acid molecules that encode 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins or biologically active portions thereof, as well as nucleic acid fragments sufficient for use as hybridization probes to identify 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-encoding nucleic acid molecules (e.g., 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA) and fragments for use as PCR primers for the amplification or mutation of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

A nucleic acid molecule used in the methods of the present invention, e.g., a nucleic acid molecule having the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 or a portion thereof, can be isolated using standard molecular biology techniques and the sequence information provided herein. Using all or portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127, as a hybridization probe, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecules can be isolated using standard hybridization and cloning techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

Moreover, a nucleic acid molecule encompassing all or a portion of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 can be isolated by the polymerase chain reaction (PCR) using synthetic oligonucleotide primers designed based upon the sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127.

A nucleic acid used in the methods of the invention can be amplified using cDNA, mRNA or, alternatively, genomic DNA as a template and appropriate oligonucleotide primers according to standard PCR amplification techniques. Furthermore, oligonucleotides corresponding to 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleotide sequences can be prepared by standard synthetic techniques, e.g., using an automated DNA synthesizer.

In a preferred embodiment, the isolated nucleic acid molecules used in the methods of the invention comprise the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127, or a complement of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 or a portion of any of these nucleotide sequences. A nucleic acid molecule which is complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 is one which is sufficiently complementary to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 such that it can hybridize to the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 thereby forming a stable duplex.

In still another preferred embodiment, an isolated nucleic acid molecule used in the methods of the present invention comprises a nucleotide sequence which is at least about 55%, 60%, 65%, 70%, 75%, 80%, 85%, 90%, 95%, 96%, 97%, 98%, 99% or more identical to the entire length of the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 or a portion of any of this nucleotide sequence.

Moreover, the nucleic acid molecules used in the methods of the invention can comprise only a portion of the nucleic acid sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 for example, a fragment which can be used as a probe or primer or a fragment encoding a portion of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, e.g., a biologically active portion of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. The probe/primer typically comprises substantially purified oligonucleotide. The oligonucleotide typically comprises a region of nucleotide sequence that hybridizes under stringent conditions to at least about 12 or 15, preferably about 20 or 25, more preferably about 30, 35, 40, 45, 50, 55, 60, 65, or 75 consecutive nucleotides of a sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 of an anti-sense sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 or of a naturally occurring allelic variant or mutant of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127. In one embodiment, a nucleic acid molecule used in the methods of the present invention comprises a nucleotide sequence which is greater than 100, 100-200, 200-300, 300-400, 400-500, 500-600, 600-700, 700-800, 800-900, 900-1000, 1000-1100, 1100-1200, 1200-1300, or more nucleotides in length and hybridizes under stringent hybridization conditions to a nucleic acid molecule of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127.

As used herein, the term “hybridizes under stringent conditions” is intended to describe conditions for hybridization and washing under which nucleotide sequences that are significantly identical or homologous to each other remain hybridized to each other. Preferably, the conditions are such that sequences at least about 70%, more preferably at least about 80%, even more preferably at least about 85% or 90% identical to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chloride/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 1×SSC plus 50% formamide at about 42-50° C.) followed by one or more washes in 0.3×SSC, at about 65-70° C. A preferred, non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.)=2(# of A+T bases)+4(# of G+C bases). For hybrids between 18 and 49 base pairs in length, Tₘ(° C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M). It will also be recognized by the skilled practitioner that additional reagents may be added to hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS), chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washes at 0.02M NaH₂PO₄, 1% SDS at 65° C., see e.g., Church and Gilbert (1984) Proc. Natl. Acad. Sci. USA 81:1991-1995, (or alternatively 0.2×SSC, 1% SDS).

In preferred embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, such as by measuring a level of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-encoding nucleic acid in a sample of cells from a subject e.g., detecting 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA levels or determining whether a genomic 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene has been mutated or deleted.

The methods of the invention further encompass the use of nucleic acid molecules that differ from the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 due to degeneracy of the genetic code and thus encode the same 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins as those encoded by the nucleotide sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127. In another embodiment, an isolated nucleic acid molecule included in the methods of the invention has a nucleotide sequence encoding a protein having an amino acid sequence shown in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127.

The methods of the invention further include the use of allelic variants of human 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419, e.g., functional and non-functional allelic variants. Functional allelic variants are naturally occurring amino acid sequence variants of the human 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein that maintain a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. Functional allelic variants will typically contain only conservative substitution of one or more amino acids of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128 or substitution, deletion or insertion of non-critical residues in non-critical regions of the protein.

Non-functional allelic variants are naturally occurring amino acid sequence variants of the human 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein that do not have a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. Non-functional allelic variants will typically contain a non-conservative substitution, deletion, or insertion or premature truncation of the amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128, or a substitution, insertion or deletion in critical residues or critical regions of the protein.

The methods of the present invention may further use non-human orthologues of the human 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Orthologues of the human 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein are proteins that are isolated from non-human organisms and possess the same 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity.

The methods of the present invention further include the use of nucleic acid molecules comprising the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 or a portion thereof, in which a mutation has been introduced. The mutation may lead to amino acid substitutions at “non-essential” amino acid residues or at “essential” amino acid residues. A “non-essential” amino acid residue is a residue that can be altered from the wild-type sequence of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 (e.g., the sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128 without altering the biological activity, whereas an “essential” amino acid residue is required for biological activity. For example, amino acid residues that are conserved among the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins of the present invention and other members of the family are not likely to be amenable to alteration.

Mutations can be introduced into SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127 by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein is preferably replaced with another amino acid residue from the same side chain family. Alternatively, in another embodiment, mutations can be introduced randomly along all or part of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 coding sequence, such as by saturation mutagenesis, and the resultant mutants can be screened for 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 biological activity to identify mutants that retain activity. Following mutagenesis of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127, the encoded protein can be expressed recombinantly and the activity of the protein can be determined using the assay described herein.

Another aspect of the invention pertains to the use of isolated nucleic acid molecules which are antisense to the nucleotide sequence of SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125 or 127. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 coding strand, or to only a portion thereof. In one embodiment, an antisense nucleic acid molecule is antisense to a “coding region” of the coding strand of a nucleotide sequence encoding a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419. The term “noncoding region” refers to 5′ and 3′ sequences which flank the coding region that are not translated into amino acids (also referred to as 5′ and 3′ untranslated regions).

Given the coding strand sequences encoding 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 disclosed herein, antisense nucleic acids of the invention can be designed according to the rules of Watson and Crick base pairing. The antisense nucleic acid molecule can be complementary to the entire coding region of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA, but more preferably is an oligonucleotide which is antisense to only a portion of the coding or noncoding region of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA. For example, the antisense oligonucleotide can be complementary to the region surrounding the translation start site of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest). Antisense nucleic acid molecules used in the methods of the invention are further described above, in section IV.

In yet another embodiment, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecules used in the methods of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-OKeefe et al. (1996) Proc. Natl. Acad. Sci. 93:14670-675.

PNAs of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecules can be used in the therapeutic and diagnostic applications described herein. For example, PNAs can be used as antisense or antigene agents for sequence-specific modulation of gene expression by, for example, inducing transcription or translation arrest or inhibiting replication. PNAs of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. et al. (1996) supra)); or as probes or primers for DNA sequencing or hybridization (Hyrup B. et al. (1996) supra; Perry-O'Keefe et al. (1996) supra).

In another embodiment, PNAs of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. et al. (1996) supra). The synthesis of PNA-DNA chimeras can be performed as described in Hyrup B. et al. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3 'PNA segment (Peterser, K. H. et al. (1975) Bioorganic Med. Chem. Lett. 5: 1119-11124).

In other embodiments, the oligonucleotide used in the methods of the invention may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO88/09810) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

VII. Isolated 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570.41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 Proteins and Anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 Antibodies Used in the Methods of the Invention

The methods of the invention include the use of isolated 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins, and biologically active portions thereof, as well as polypeptide fragments suitable for use as immunogens to raise anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies. In one embodiment, native 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins can be isolated from cells or tissue sources by an appropriate purification scheme using standard protein purification techniques. In another embodiment, 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins are produced by recombinant DNA techniques. Alternative to recombinant expression, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or polypeptide can be synthesized chemically using standard peptide synthesis techniques.

As used herein, a “biologically active portion” of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein includes a fragment of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein having a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity. Biologically active portions of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein include peptides comprising amino acid sequences sufficiently identical to or derived from the amino acid sequence of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, e.g., the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128, which include fewer amino acids than the full length 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins, and exhibit at least one activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Typically, biologically active portions comprise a domain or motif with at least one activity of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein (e.g., the N-terminal region of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein that is believed to be involved in the regulation of apoptotic activity). A biologically active portion of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be a polypeptide which is, for example, 25, 50, 75, 100, 125, 150, 175, 200, 250, 300 or more amino acids in length. Biologically active portions of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be used as targets for developing agents which modulate a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 activity.

In a preferred embodiment, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein used in the methods of the invention has an amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128. In other embodiments, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein is substantially identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128 and retains the functional activity of the protein of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128, yet differs in amino acid sequence due to natural allelic variation or mutagenesis, as described in detail in subsection V above. Accordingly, in another embodiment, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein used in the methods of the invention is a protein which comprises an amino acid sequence at least about 50%, 55%, 60%, 65%, 70%, 75%, 80%, 85%, 90%, 95%, 96%, 97%, 98%, 99% or more identical to SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 amino acid sequence of SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128, having 500 amino acid residues, at least 75, preferably at least 150, more preferably at least 225, even more preferably at least 300, and even more preferably at least 400 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. 48:444-453 (1970)) algorithm which has been incorporated into the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (Comput. Appl. Biosci. 4:11-17 (1988)) which has been incorporated into the ALIGN program (version 2.0 or 2.0U), using a PAM120 weight residue table, a gap length penalty of 12 and a gap penalty of 4.

The methods of the invention may also use 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 chimeric or fusion proteins. As used herein, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 “chimeric protein” or “fusion protein” comprises a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide operatively linked to a non-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide. An “1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide” refers to a polypeptide having an amino acid sequence corresponding to a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 molecule, whereas a “non-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide” refers to a polypeptide having an amino acid sequence corresponding to a protein which is not substantially homologous to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, e.g., a protein which is different from the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein and which is derived from the same or a different organism. Within a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion protein the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide can correspond to all or a portion of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. In a preferred embodiment, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion protein comprises at least one biologically active portion of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. In another preferred embodiment, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion protein comprises at least two biologically active portions of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Within the fusion protein, the term “operatively linked” is intended to indicate that the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide and the non-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide are fused in-frame to each other. The non-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide can be fused to the N-terminus or C-terminus of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide.

For example, in one embodiment, the fusion protein is a GST-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion protein in which the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequences are fused to the C-terminus of the GST sequences. Such fusion proteins can facilitate the purification of recombinant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419.

In another embodiment, this fusion protein is a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein containing a heterologous signal sequence at its N-terminus. In certain host cells (e.g., mammalian host cells), expression and/or secretion of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be increased through use of a heterologous signal sequence.

The 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion proteins used in the methods of the invention can be incorporated into pharmaceutical compositions and administered to a subject in vivo. The 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion proteins can be used to affect the bioavailability of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate. Use of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fusion proteins may be useful therapeutically for the treatment of disorders caused by, for example, (i) aberrant modification or mutation of a gene encoding a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein; (ii) mis-regulation of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 gene; and (iii) aberrant post-translational modification of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein.

Moreover, the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-fusion proteins used in the methods of the invention can be used as immunogens to produce anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies in a subject, to purify 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 ligands and in screening assays to identify molecules which inhibit the interaction of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 substrate.

Preferably, a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 chimeric or fusion protein used in the methods of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-encoding nucleic acid can be cloned into such an expression vector such that the fusion moiety is linked in-frame to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein.

The present invention also pertains to the use of variants of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins which function as either 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 agonists (mimetics) or as 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antagonists. Variants of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins can be generated by mutagenesis, e.g., discrete point mutation or truncation of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. An agonist of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins can retain substantially the same, or a subset, of the biological activities of the naturally occurring form of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. An antagonist of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can inhibit one or more of the activities of the naturally occurring form of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein by, for example, competitively modulating a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419-mediated activity of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein.

In one embodiment, variants of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein which function as either 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 agonists (mimetics) or as 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antagonists can be identified by screening combinatorial libraries of mutants, e.g., truncation mutants, of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein for 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein agonist or antagonist activity. In one embodiment, a variegated library of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 variants is generated by combinatorial mutagenesis at the nucleic acid level and is encoded by a variegated gene library. A variegated library of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 variants can be produced by, for example, enzymatically ligating a mixture of synthetic oligonucleotides into gene sequences such that a degenerate set of potential 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequences is expressible as individual polypeptides, or alternatively, as a set of larger fusion proteins (e.g., for phage display) containing the set of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequences therein. There are a variety of methods which can be used to produce libraries of potential 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein coding sequence can be used to generate a variegated population of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 fragments for screening and subsequent selection of variants of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. In one embodiment, a library of coding sequence fragments can be generated by treating a double stranded PCR fragment of a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 variants (Arkin and Yourvan (1992) Proc. Natl. Acad. Sci. USA 89:7811-7815; Delgrave et al. (1993) Protein Engineering 6(3):327-331).

The methods of the present invention further include the use of anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies. An isolated 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein, or a portion or fragment thereof, can be used as an immunogen to generate antibodies that bind 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 using standard techniques for polyclonal and monoclonal antibody preparation. A full-length 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein can be used or, alternatively, antigenic peptide fragments of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be used as immunogens. The antigenic peptide of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 comprises at least 8 amino acid residues of the amino acid sequence shown in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126 or 128 and encompasses an epitope of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 such that an antibody raised against the peptide forms a specific immune complex with the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 that are located on the surface of the protein, e.g., hydrophilic regions, as well as regions with high antigenicity.

A 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 immunogen is typically used to prepare antibodies by immunizing a suitable subject, (e.g., rabbit, goat, mouse, or other mammal) with the immunogen. An appropriate immunogenic preparation can contain, for example, recombinantly expressed 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein or a chemically synthesized 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide. The preparation can further include an adjuvant, such as Freund's complete or incomplete adjuvant, or similar immunostimulatory agent. Immunization of a suitable subject with an immunogenic 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 preparation induces a polyclonal anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody response.

The term “antibody” as used herein refers to immunoglobulin molecules and immunologically active portions of immunoglobulin molecules, i.e., molecules that contain an antigen binding site which specifically binds (immunoreacts with) an antigen, such as a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419. Examples of immunologically active portions of immunoglobulin molecules include F(ab) and F(ab)₂ fragments which can be generated by treating the antibody with an enzyme such as pepsin. The invention provides polyclonal and monoclonal antibodies that bind 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 molecules. The term “monoclonal antibody” or “monoclonal antibody composition”, as used herein, refers to a population of antibody molecules that contain only one species of an antigen binding site capable of immunoreacting with a particular epitope of 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419. A monoclonal antibody composition thus typically displays a single binding affinity for a particular 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein with which it immunoreacts.

Polyclonal anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies can be prepared as described above by immunizing a suitable subject with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 immunogen. The anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody titer in the immunized subject can be monitored over time by standard techniques, such as with an enzyme linked immunosorbent assay (ELISA) using immobilized 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419. If desired, the antibody molecules directed against 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 can be isolated from the mammal (e.g., from the blood) and further purified by well known techniques, such as protein A chromatography to obtain the IgG fraction. At an appropriate time after immunization, e.g., when the anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 immunogen as described above, and the culture supernatants of the resulting hybridoma cells are screened to identify a hybridoma producing a monoclonal antibody that binds 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419.

Any of the many well known protocols used for fusing lymphocytes and immortalized cell lines can be applied for the purpose of generating an anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 monoclonal antibody (see, e.g., G. Galfre et al. (1977) Nature 266:55052; Gefter et al. (1977) supra; Lerner (1981) supra; and Kenneth (1980) supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any ofa number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419, e.g., using a standard ELISA assay.

Alternative to preparing monoclonal antibody-secreting hybridomas, a monoclonal anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody can be identified and isolated by screening a recombinant combinatorial immunoglobulin library (e.g., an antibody phage display library) with 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 to thereby isolate immunoglobulin library members that bind 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrad et al. (1991) Bio/Technology 9:1373-1377; Hoogenboom et al. (1991) Nuc. Acid Res. 19:4133-4137; Barbas et al. (1991) Proc. Natl. Acad. Sci. USA 88:7978-7982; and McCafferty et al. (1990) Nature 348:552-554.

Additionally, recombinant anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies, such as chimeric and humanized monoclonal antibodies, comprising both human and non-human portions, which can be made using standard recombinant DNA techniques, are within the scope of the methods of the invention. Such chimeric and humanized monoclonal antibodies can be produced by recombinant DNA techniques known in the art, for example using methods described in Robinson et al. International Application No. PCT/US86/02269; Akira, et al. European Patent Application 184, 187; Taniguchi, M., European Patent Application 171, 496; Morrison et al. European Patent Application 173, 494; Neuberger et al. PCT International Publication No. WO 86/01533; Cabilly et al. U.S. Pat. No. 4,816,567; Cabilly et al. European Patent Application 125, 023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; Shaw et al. (1988) J. Natl. Cancer Inst. 80:1553-1559; Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:40534060.

An anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody can be used to detect 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein (e.g., in a cellular lysate or cell supernatant) in order to evaluate the abundance and pattern of expression of the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 protein. Anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

This invention is further illustrated by the following examples which should not be construed as limiting. The contents of all references, patents and published patent applications cited throughout this application, as well as the Figure and the Sequence Listing is incorporated herein by reference.

### EXAMPLES

### EXAMPLE 1

### TISSUE DISTRIBUTION OF USING TAQMAN™ ANALYSIS

This example describes the TaqMan™ procedure. The Taqman™ procedure is a quantitative, reverse transcription PCR-based approach for detecting mRNA. The RT-PCR reaction exploits the 5′ nuclease activity of AmpliTaq Gold™ DNA Polymerase to cleave a TaqMan™ probe during PCR. Briefly, cDNA was generated from the samples of interest, e.g., heart, kidney, liver, skeletal muscle, and various vessels, and used as the starting material for PCR amplification. In addition to the 5′ and 3′ gene-specific primers, a gene-specific oligonucleotide probe (complementary to the region being amplified) was included in the reaction (i.e., the Taqman™ probe). The TaqMan™ probe includes the oligonucleotide with a fluorescent reporter dye covalently linked to the 5′ end of the probe (such as FAM (6-carboxyfluorescein), TET (6-carboxy4,7,2′,7′-tetrachlorofluorescein), JOE (6-carboxy-4,5-dichloro-2,7-dimethoxyfluorescein), or VIC) and a quencher dye (TAMRA (6-carboxy-N,N,N′,N′-tetramethylrhodamine) at the 3′ end of the probe.

During the PCR reaction, cleavage of the probe separates the reporter dye and the quencher dye, resulting in increased fluorescence of the reporter. Accumulation of PCR products is detected directly by monitoring the increase in fluorescence of the reporter dye. When the probe is intact, the proximity of the reporter dye to the quencher dye results in suppression of the reporter fluorescence. During PCR, if the target of interest is present, the probe specifically anneals between the forward and reverse primer sites. The 5′-3′ nucleolytic activity of the AmpliTaq™ Gold DNA Polymerase cleaves the probe between the reporter and the quencher only if the probe hybridizes to the target. The probe fragments are then displaced from the target, and polymerization of the strand continues. The 3′ end of the probe is blocked to prevent extension of the probe during PCR. This process occurs in every cycle and does not interfere with the exponential accumulation of product. RNA was prepared using the trizol method and treated with DNase to remove contaminating genomic DNA. cDNA was synthesized using standard techniques. Mock cDNA synthesis in the absence of reverse transcriptase resulted in samples with no detectable PCR amplification of the control gene confirms efficient removal of genomic DNA contamination.

### EXAMPLE 2

### TISSUE DISTRIBUTION OF USING 1N SITU ANALYSIS

For in situ analysis, various tissues, e.g., tissues obtained from normal colon, breast, lung, and ovarian normal tissue, as well as colon, breast, lung, and ovarian tumors, colon metastatic to the liver, and angiogenic tissues were first frozen on dry ice. Ten-micrometer-thick sections of the tissues were post-fixed with 4% formaldehyde in DEPC treated 1× phosphate-buffered saline at room temperature for 10 minutes before being rinsed twice in DEPC 1× phosphate-buffered saline and once in 0.1 M triethanolamine-HCl (pH 8.0). Following incubation in 0.25% acetic anhydride-0.1 M triethanolamine-HCl for 10 minutes, sections were rinsed in DEPC 2×SSC (1×SSC is 0.15M NaCl plus 0.015M sodium citrate). Tissue was then dehydrated through a series of ethanol washes, incubated in 100% chloroform for 5 minutes, and then rinsed in 100% ethanol for 1 minute and 95% ethanol for 1 minute and allowed to air dry.

Hybridizations were performed with ³⁵S-radiolabeled (5×10⁷ cpm/ml) cRNA probes. Probes were incubated in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1× Denhardt's solution, 50% formamide, 10% dextran sulfate, 100 mM dithiothreitol, 0.1% sodium dodecyl sulfate (SDS), and 0.1% sodium thiosulfate for 18 hours at 55° C.

After hybridization, slides were washed with 2×SSC. Sections were then sequentially incubated at 37° C. in TNE (a solution containing 10 mM Tris-HCl (pH 7.6), 500 mM NaCl, and 1 mM EDTA), for 10 minutes, in TNE with 10 μg of RNase A per ml for 30 minutes, and finally in TNE for 10 minutes. Slides were then rinsed with 2×SSC at room temperature, washed with 2×SSC at 50° C. for 1 hour, washed with 0.2×SSC at 55° C. for 1 hour, and 0.2×SSC at 60° C. for 1 hour. Sections were then dehydrated rapidly through serial ethanol-0.3 M sodium acetate concentrations before being air dried and exposed to Kodak Biomax MR scientific imaging film for 24 hours and subsequently dipped in NB-2 photoemulsion and exposed at 4° C. for 7 days before being developed and counter stained.

Equivalents:

Those skilled in the art will recognize, or be able to ascertain using no more than routine experimentation, many equivalents to the specific embodiments of the invention described herein. Such equivalents are intended to be encompassed by the following claims.

1. A method for identifying a compound capable of treating a cardiovascular disorder or a thrombotic disorder, comprising:
a) combining a compound to be tested with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide under conditions suitable for binding of the test compound to the polypeptide; and
b) detecting binding of the test compound to the polypeptide to thereby identify a compound which binds to the polypeptide,
thereby identifying a compound capable of treating a cardiovascular disorder or a thrombotic disorder:.

2. The method of claim 1, wherein the compound is selected from the group consisting of a small molecule, a peptide or an antibody.

3. The method of claim 1, wherein the polypeptide further comprises heterologous sequences.

4. The method of claim 1, wherein the polypeptide is an isolated polypeptide, a membrane-bound form of an isolated polypeptide or a cell comprising the polypeptide.

5. The method of claim 1, wherein the disorder is aberrant vascularization, atherosclerosis, thrombosis, coronary artery disease, hyperlipidemia, dyslipidemia, high blood pressure and heart failure.

6. The method of claim 1, wherein the binding of the test compound to the polypeptide is detected by a method selected from the group consisting of:
a) a competition binding assay;
b) an immunoassay; and
c) a yeast two-hybrid assay.

7. A method for identifying a compound capable of treating a cardiovascular disorder or a thrombotic disorder, comprising:
a) combining a compound to be tested with a host cell expressing a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide under conditions suitable for binding of the test compound to the polypeptide; and
b) detecting binding of the test compound to the polypeptide to thereby identify a compound which binds to the polypeptide,
thereby identifying a compound capable of treating a cardiovascular disorder or a thrombotic disorder.

8. The method of claim 7, wherein the compound is selected for the group consisting of a small molecule, a peptide, an antibody or an antisense nucleic acid molecule.

9. The method of claim 7, wherein the polypeptide further comprises heterologous sequences.

10. The method of claim 7, wherein the disorder is aberrant vascularization, atherosclerosis, thrombosis, coronary artery disease, hyperlipidemia, dyslipidemia, high blood pressure and heart failure.

11. The method of claim 7, wherein the binding of the test compound to the polypeptide is detected by a method selected from the group consisting of:
a) a competition binding assay;
b) an immunoassay; and
c) a yeast two-hybrid assay.

12. A method of identifying a subject having a cardiovascular or thrombotic disorder, or at risk for developing a cardiovascular or thrombotic disorder comprising:
a) contacting a sample obtained from the subject comprising polypeptides with a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 binding substance; and
b) detecting the presence of a polypeptide in the sample that binds to the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 binding substance, thereby identifying a subject having a cardiovascular or thrombotic disorder, or at risk for developing a cardiovascular or thrombotic disorder.

13. The method of claim 12, wherein the binding substance is an antibody.

14. The method of claim 12, wherein the binding substance is detectably labeled.

15. A method for treating a subject having a cardiovascular or thrombotic disorder characterized by aberrant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide activity or aberrant 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid expression comprising administering to the subject a 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator, thereby treating the subject having a cardiovascular or thrombotic disorder.

16. The method of claim 15, wherein the disorder is aberrant vascularization, atherosclerosis, thrombosis, coronary artery disease, hyperlipidemia, dyslipidemia, high blood pressure and heart failure.

17. The method of claim 15, wherein the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator is administered in a pharmaceutically acceptable formulation.

18. The method of 15, wherein the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator is capable of modulating 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 polypeptide activity.

19. The method of claim 18, wherein the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator is an anti-1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 antibody.

20. The method of claim 15, wherein the 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 modulator is capable of modulating 1722, 10280, 59917, 85553, 10653, 9235, 21668, 17794, 2210, 6169, 10102, 21061, 17662, 1468, 12282, 6350, 9035, 1820, 23652, 7301, 8925, 8701, 3533, 9462, 9123, 12788, 17729, 65552, 1261, 21476, 33770, 9380, 2569654, 33556, 53656, 44143, 32612, 10671, 261, 44570, 41922, 2552, 2417, 19319, 43969, 8921, 8993, 955, 32345, 966, 1920, 17318, 1510, 14180, 26005, 554, 16408, 42028, 112091, 13886, 13942, 1673, 54946 or 2419 nucleic acid expression.
